SEQUENCE LISTING (411 sequences)

SEQ ID NO: 1 - DNA, 21 bp, Homo sapiens
  misc_feature (1)..(21): 5′ sense dopamine-beta-hydroxylase target protein 5′ DNA sense
gtccctggagctctcatggaa

SEQ ID NO: 2 - DNA, 21 bp, Homo sapiens
  misc_feature (1)..(21): 5′ sense dopamine-beta-hydroxylase target protein 5′ DNA sense
aatgtcagctacacccaggag

SEQ ID NO: 3 - DNA, 21 bp, Homo sapiens
  misc_feature (1)..(21): 5′ sense dopamine-beta-hydroxylase target protein 5′ DNA sense
gctcctggtgcggaggctcaa

SEQ ID NO: 4 - DNA, 21 bp, Homo sapiens
  misc_feature (1)..(21): 5′ sense dopamine-beta-hydroxylase target protein 5′ DNA sense
aaggctggcgtcctgtttggg

SEQ ID NO: 5 - DNA, 25 bp, Homo sapiens
  misc_feature (1)..(25): 5′ sense dopamine-beta-hydroxylase target protein 5′ DNA sense
tgtccgaccgtggcgagcttgagaa

SEQ ID NO: 6 - DNA, 23 bp, Homo sapiens
  misc_feature (1)..(23): 5′ sense dopamine-beta-hydroxylase target protein 5′ DNA sense
tccgaccgtggcgagcttgagaa

SEQ ID NO: 7 - DNA, 21 bp, Homo sapiens
  misc_feature (1)..(21): 5′ sense dopamine-beta-hydroxylase target protein 5′ DNA sense
cgaccgtggcgagcttgagaa

SEQ ID NO: 8 - DNA, 19 bp, Homo sapiens
  misc_feature (1)..(19): 5′ sense dopamine-beta-hydroxylase target protein 5′ DNA sense
accgtggcgagcttgagaa

SEQ ID NO: 9 - DNA, 21 bp, Homo sapiens
  misc_feature (1)..(21): 5′ sense dopamine-beta-hydroxylase target protein 5′ DNA sense
aacgcagatctcgtggtgctc

SEQ ID NO: 10 - DNA, 21 bp, Homo sapiens
  misc_feature (1)..(21): 5′ sense dopamine-beta-hydroxylase target protein 5′ DNA sense
ggacgcctggagtgaccagaa

SEQ ID NO: 11 - DNA, 21 bp, Homo sapiens
  misc_feature (1)..(21): 5′ sense dopamine-beta-hydroxylase target protein 5′ DNA sense
aaggggcagatccacctggat

SEQ ID NO: 12 - DNA, 21 bp, Homo sapiens
  misc_feature (1)..(21): 5′ sense dopamine-beta-hydroxylase target protein 5′ DNA sense
caggtgcagaggaccccagaa

SEQ ID NO: 13 - DNA, 21 bp, Homo sapiens
  misc_feature (1)..(21): 5′ sense dopamine-beta-hydroxylase target protein 5′ DNA sense
aaggcctgaccctgcttttca

SEQ ID NO: 14 - DNA, 19 bp, Homo sapiens
  misc_feature (1)..(19): 5′ sense dopamine-beta-hydroxylase target protein 5′ DNA sense
aaggcctgaccctgctttt

SEQ ID NO: 15 - DNA, 23 bp, Homo sapiens
  misc_feature (1)..(23): 5′ sense dopamine-beta-hydroxylase target protein 5′ DNA sense
aaggcctgaccctgcttttcaag

SEQ ID NO: 16 - DNA, 25 bp, Homo sapiens
  misc_feature (1)..(25): 5′ sense dopamine-beta-hydroxylase target protein 5′ DNA sense
aaggcctgaccctgcttttcaagag

SEQ ID NO: 17 - DNA, 21 bp, Homo sapiens
  misc_feature (1)..(21): 5′ sense dopamine-beta-hydroxylase target protein 5′ DNA sense
aggcctgaccctgcttttcaa

SEQ ID NO: 18 - DNA, 21 bp, Homo sapiens
  misc_feature (1)..(21): 5′ sense dopamine-beta-hydroxylase target protein 5′ DNA sense
aagaggccctttggcacctgc

SEQ ID NO: 19 - DNA, 21 bp, Homo sapiens
  misc_feature (1)..(21): 5′ sense dopamine-beta-hydroxylase target protein 5′ DNA sense
ctttggcacctgcgaccccaa

SEQ ID NO: 20 - DNA, 21 bp, Homo sapiens
  misc_feature (1)..(21): 5′ sense dopamine-beta-hydroxylase target protein 5′ DNA sense
cccaaggattacctcattgaa

SEQ ID NO: 21 - DNA, 21 bp, Homo sapiens
  misc_feature (1)..(21): 5′ sense dopamine-beta-hydroxylase target protein 5′ DNA sense
aaggattacctcattgaagac

SEQ ID NO: 22 - DNA, 21 bp, Homo sapiens
  misc_feature (1)..(21): 5′ sense dopamine-beta-hydroxylase target protein 5′ DNA sense
aagacggcactgtccacttgg

SEQ ID NO: 23 - DNA, 21 bp, Homo sapiens
  misc_feature (1)..(21): 5′ sense dopamine-beta-hydroxylase target protein 5′ DNA sense
ccggtcactggaggccatcaa

SEQ ID NO: 24 - DNA, 21 bp, Homo sapiens
  misc_feature (1)..(21): 5′ sense dopamine-beta-hydroxylase target protein 5′ DNA sense
aacggctcgggcctgcagatg

SEQ ID NO: 25 - DNA, 21 bp, Homo sapiens
  misc_feature (1)..(21): 5′ sense dopamine-beta-hydroxylase target protein 5′ DNA sense
gcagagggtgcagctcctgaa

SEQ ID NO: 26 - DNA, 21 bp, Homo sapiens
  misc_feature (1)..(21): 5′ sense dopamine-beta-hydroxylase target protein 5′ DNA sense
ggtgcagctcctgaagcccaa

SEQ ID NO: 27 - DNA, 21 bp, Homo sapiens
  misc_feature (1)..(21): 5′ sense dopamine-beta-hydroxylase target protein 5′ DNA sense
ctgaagcccaatatccccgaa

SEQ ID NO: 28 - DNA, 21 bp, Homo sapiens
  misc_feature (1)..(21): 5′ sense dopamine-beta-hydroxylase target protein 5′ DNA sense
aagcccaatatccccgaaccg

SEQ ID NO: 29 - DNA, 21 bp, Homo sapiens
  misc_feature (1)..(21): 5′ sense dopamine-beta-hydroxylase target protein 5′ DNA sense
aatatccccgaaccggagttg

SEQ ID NO: 30 - DNA, 21 bp, Homo sapiens
  misc_feature (1)..(21): 5′ sense dopamine-beta-hydroxylase target protein 5′ DNA sense
aaccggagttgccctcagacg

SEQ ID NO: 31 - DNA, 21 bp, Homo sapiens
  misc_feature (1)..(21): 5′ sense dopamine-beta-hydroxylase target protein 5′ DNA sense
gcgtgcaccatggaggtccaa

SEQ ID NO: 32 - DNA, 21 bp, Homo sapiens
  misc_feature (1)..(21): 5′ sense dopamine-beta-hydroxylase target protein 5′ DNA sense
catggaggtccaagctcccaa

SEQ ID NO: 33 - DNA, 21 bp, Homo sapiens
  misc_feature (1)..(21): 5′ sense dopamine-beta-hydroxylase target protein 5′ DNA sense
aagctcccaatatccagatcc

SEQ ID NO: 34 - DNA, 21 bp, Homo sapiens
  misc_feature (1)..(21): 5′ sense dopamine-beta-hydroxylase target protein 5′ DNA sense
aatatccagatccccagccag

SEQ ID NO: 35 - DNA, 25 bp, Homo sapiens
  misc_feature (1)..(25): 5′ sense dopamine-beta-hydroxylase target protein 5′ DNA sense
agaccacgtactggtgctacattaa

SEQ ID NO: 36 - DNA, 23 bp, Homo sapiens
  misc_feature (1)..(23): 5′ sense dopamine-beta-hydroxylase target protein 5′ DNA sense
accacgtactggtgctacattaa

SEQ ID NO: 37 - DNA, 21 bp, Homo sapiens
  misc_feature (1)..(21): 5′ sense dopamine-beta-hydroxylase target protein 5′ DNA sense
cacgtactggtgctacattaa

SEQ ID NO: 38 - DNA, 19 bp, Homo sapiens
  misc_feature (1)..(19): 5′ sense dopamine-beta-hydroxylase target protein 5′ DNA sense
cgtactggtgctacattaa

SEQ ID NO: 39 - DNA, 21 bp, Homo sapiens
  misc_feature (1)..(21): 5′ sense dopamine-beta-hydroxylase target protein 5′ DNA sense
gctacattaaggagcttccaa

SEQ ID NO: 40 - DNA, 21 bp, Homo sapiens
  misc_feature (1)..(21): 5′ sense dopamine-beta-hydroxylase target protein 5′ DNA sense
ctacattaaggagcttccaaa

SEQ ID NO: 41 - DNA, 21 bp, Homo sapiens
  misc_feature (1)..(21): 5′ sense dopamine-beta-hydroxylase target protein 5′ DNA sense
aaggagcttccaaagggcttc

SEQ ID NO: 42 - DNA, 21 bp, Homo sapiens
  misc_feature (1)..(21): 5′ sense dopamine-beta-hydroxylase target protein 5′ DNA sense
aaagggcttctctcggcacca

SEQ ID NO: 43 - DNA, 21 bp, Homo sapiens
  misc_feature (1)..(21): 5′ sense dopamine-beta-hydroxylase target protein 5′ DNA sense
aagggcttctctcggcaccac

SEQ ID NO: 44 - DNA, 25 bp, Homo sapiens
  misc_feature (1)..(25): 5′ sense dopamine-beta-hydroxylase target protein 5′ DNA sense
gcttctctcggcaccacattatcaa

SEQ ID NO: 45 - DNA, 23 bp, Homo sapiens
  misc_feature (1)..(23): 5′ sense dopamine-beta-hydroxylase target protein 5′ DNA sense
ttctctcggcaccacattatcaa

SEQ ID NO: 46 - DNA, 21 bp, Homo sapiens
  misc_feature (1)..(21): 5′ sense dopamine-beta-hydroxylase target protein 5′ DNA sense
ctctcggcaccacattatcaa

SEQ ID NO: 47 - DNA, 19 bp, Homo sapiens
  misc_feature (1)..(19): 5′ sense dopamine-beta-hydroxylase target protein 5′ DNA sense
ctcggcaccacattatcaa

SEQ ID NO: 48 - DNA, 21 bp, Homo sapiens
  misc_feature (1)..(21): 5′ sense dopamine-beta-hydroxylase target protein 5′ DNA sense
aagtacgagcccatcgtcacc

SEQ ID NO: 49 - DNA, 21 bp, Homo sapiens
  misc_feature (1)..(21): 5′ sense dopamine-beta-hydroxylase target protein 5′ DNA sense
gtacgagcccatcgtcaccaa

SEQ ID NO: 50 - DNA, 21 bp, Homo sapiens
  misc_feature (1)..(21): 5′ sense dopamine-beta-hydroxylase target protein 5′ DNA sense
gcccatcgtcaccaagggcaa

SEQ ID NO: 51 - DNA, 21 bp, Homo sapiens
  misc_feature (1)..(21): 5′ sense dopamine-beta-hydroxylase target protein 5′ DNA sense
aagggcaatgaggcccttgtc

SEQ ID NO: 52 - DNA, 21 bp, Homo sapiens
  misc_feature (1)..(21): 5′ sense dopamine-beta-hydroxylase target protein 5′ DNA sense
aatgaggcccttgtccaccac

SEQ ID NO: 53 - DNA, 21 bp, Homo sapiens
  misc_feature (1)..(21): 5′ sense dopamine-beta-hydroxylase target protein 5′ DNA sense
gcccttgtccaccacatggaa

SEQ ID NO: 54 - DNA, 21 bp, Homo sapiens
  misc_feature (1)..(21): 5′ sense dopamine-beta-hydroxylase target protein 5′ DNA sense
aagtcttccagtgcgcccccg

SEQ ID NO: 55 - DNA, 21 bp, Homo sapiens
  misc_feature (1)..(21): 5′ sense dopamine-beta-hydroxylase target protein 5′ DNA sense
cagcgggccctgcgactccaa

SEQ ID NO: 56 - DNA, 25 bp, Homo sapiens
  misc_feature (1)..(25): 5′ sense dopamine-beta-hydroxylase target protein 5′ DNA sense
cgggccctgcgactccaagatgaaa

SEQ ID NO: 57 - DNA, 23 bp, Homo sapiens
  misc_feature (1)..(23): 5′ sense dopamine-beta-hydroxylase target protein 5′ DNA sense
ggccctgcgactccaagatgaaa

SEQ ID NO: 58 - DNA, 21 bp, Homo sapiens
  misc_feature (1)..(21): 5′ sense dopamine-beta-hydroxylase target protein 5′ DNA sense
gccctgcgactccaagatgaa

SEQ ID NO: 59 - DNA, 21 bp, Homo sapiens
  misc_feature (1)..(21): 5′ sense dopamine-beta-hydroxylase target protein 5′ DNA sense
ccctgcgactccaagatgaaa

SEQ ID NO: 60 - DNA, 19 bp, Homo sapiens
  misc_feature (1)..(19): 5′ sense dopamine-beta-hydroxylase target protein 5′ DNA sense
ctgcgactccaagatgaaa

SEQ ID NO: 61 - DNA, 21 bp, Homo sapiens
  misc_feature (1)..(21): 5′ sense dopamine-beta-hydroxylase target protein 5′ DNA sense
aagatgaaacccgaccgcctc

SEQ ID NO: 62 - DNA, 21 bp, Homo sapiens
  misc_feature (1)..(21): 5′ sense dopamine-beta-hydroxylase target protein 5′ DNA sense
gatgaaacccgaccgcctcaa

SEQ ID NO: 63 - DNA, 21 bp, Homo sapiens
  misc_feature (1)..(21): 5′ sense dopamine-beta-hydroxylase target protein 5′ DNA sense
aaacccgaccgcctcaactac

SEQ ID NO: 64 - DNA, 21 bp, Homo sapiens
  misc_feature (1)..(21): 5′ sense dopamine-beta-hydroxylase target protein 5′ DNA sense
aacccgaccgcctcaactact

SEQ ID NO: 65 - DNA, 21 bp, Homo sapiens
  misc_feature (1)..(21): 5′ sense dopamine-beta-hydroxylase target protein 5′ DNA sense
aactactgccgccacgtgctg

SEQ ID NO: 66 - DNA, 21 bp, Homo sapiens
  misc_feature (1)..(21): 5′ sense dopamine-beta-hydroxylase target protein 5′ DNA sense
cgcctgggccctgggtgccaa

SEQ ID NO: 67 - DNA, 21 bp, Homo sapiens
  misc_feature (1)..(21): 5′ sense dopamine-beta-hydroxylase target protein 5′ DNA sense
aaggcattttactacccagag

SEQ ID NO: 68 - DNA, 21 bp, Homo sapiens
  misc_feature (1)..(21): 5′ sense dopamine-beta-hydroxylase target protein 5′ DNA sense
gcattttactacccagaggaa

SEQ ID NO: 69 - DNA, 21 bp, Homo sapiens
  misc_feature (1)..(21): 5′ sense dopamine-beta-hydroxylase target protein 5′ DNA sense
aagccggccttgccttcgggg

SEQ ID NO: 70 - DNA, 21 bp, Homo sapiens
  misc_feature (1)..(21): 5′ sense dopamine-beta-hydroxylase target protein 5′ DNA sense
tccagatatctccgcctggaa

SEQ ID NO: 71 - DNA, 21 bp, Homo sapiens
  misc_feature (1)..(21): 5′ sense dopamine-beta-hydroxylase target protein 5′ DNA sense
cctggaagttcactaccacaa

SEQ ID NO: 72 - DNA, 21 bp, Homo sapiens
  misc_feature (1)..(21): 5′ sense dopamine-beta-hydroxylase target protein 5′ DNA sense
aagttcactaccacaacccac

SEQ ID NO: 73 - DNA, 21 bp, Homo sapiens
  misc_feature (1)..(21): 5′ sense dopamine-beta-hydroxylase target protein 5′ DNA sense
cacaacccactggtgatagaa

SEQ ID NO: 74 - DNA, 21 bp, Homo sapiens
  misc_feature (1)..(21): 5′ sense dopamine-beta-hydroxylase target protein 5′ DNA sense
aacccactggtgatagaagga

SEQ ID NO: 75 - DNA, 21 bp, Homo sapiens
  misc_feature (1)..(21): 5′ sense dopamine-beta-hydroxylase target protein 5′ DNA sense
cactggtgatagaaggacgaa

SEQ ID NO: 76 - DNA, 21 bp, Homo sapiens
  misc_feature (1)..(21): 5′ sense dopamine-beta-hydroxylase target protein 5′ DNA sense
actggtgatagaaggacgaaa

SEQ ID NO: 77 - DNA, 21 bp, Homo sapiens
  misc_feature (1)..(21): 5′ sense dopamine-beta-hydroxylase target protein 5′ DNA sense
aaggacgaaacgactcctcag

SEQ ID NO: 78 - DNA, 21 bp, Homo sapiens
  misc_feature (1)..(21): 5′ sense dopamine-beta-hydroxylase target protein 5′ DNA sense
aaacgactcctcaggcatccg

SEQ ID NO: 79 - DNA, 21 bp, Homo sapiens
  misc_feature (1)..(21): 5′ sense dopamine-beta-hydroxylase target protein 5′ DNA sense
aacgactcctcaggcatccgc

SEQ ID NO: 80 - DNA, 21 bp, Homo sapiens
  misc_feature (1)..(21): 5′ sense dopamine-beta-hydroxylase target protein 5′ DNA sense
ccgcttgtactacacagccaa

SEQ ID NO: 81 - DNA, 21 bp, Homo sapiens
  misc_feature (1)..(21): 5′ sense dopamine-beta-hydroxylase target protein 5′ DNA sense
agccaagctgcggcgcttcaa

SEQ ID NO: 82 - DNA, 21 bp, Homo sapiens
  misc_feature (1)..(21): 5′ sense dopamine-beta-hydroxylase target protein 5′ DNA sense
aagctgcggcgcttcaacgcg

SEQ ID NO: 83 - DNA, 21 bp, Homo sapiens
  misc_feature (1)..(21): 5′ sense dopamine-beta-hydroxylase target protein 5′ DNA sense
aacgcggggatcatggagctg

SEQ ID NO: 84 - DNA, 21 bp, Homo sapiens
  misc_feature (1)..(21): 5′ sense dopamine-beta-hydroxylase target protein 5′ DNA sense
cactggctactgcacggacaa

SEQ ID NO: 85 - DNA, 21 bp, Homo sapiens
  misc_feature (1)..(21): 5′ sense dopamine-beta-hydroxylase target protein 5′ DNA sense
aagtgcacccagctggcactg

SEQ ID NO: 86 - DNA, 21 bp, Homo sapiens
  misc_feature (1)..(21): 5′ sense dopamine-beta-hydroxylase target protein 5′ DNA sense
acacacacctgactgggagaa

SEQ ID NO: 87 - DNA, 21 bp, Homo sapiens
  misc_feature (1)..(21): 5′ sense dopamine-beta-hydroxylase target protein 5′ DNA sense
cacacacctgactgggagaaa

SEQ ID NO: 88 - DNA, 21 bp, Homo sapiens
  misc_feature (1)..(21): 5′ sense dopamine-beta-hydroxylase target protein 5′ DNA sense
aaaggtggtcacagtgctggt

SEQ ID NO: 89 - DNA, 21 bp, Homo sapiens
  misc_feature (1)..(21): 5′ sense dopamine-beta-hydroxylase target protein 5′ DNA sense
aaggtggtcacagtgctggtc

SEQ ID NO: 90 - DNA, 21 bp, Homo sapiens
  misc_feature (1)..(21): 5′ sense dopamine-beta-hydroxylase target protein 5′ DNA sense
ccgggagtgggagatcgtgaa

SEQ ID NO: 91 - DNA, 21 bp, Homo sapiens
  misc_feature (1)..(21): 5′ sense dopamine-beta-hydroxylase target protein 5′ DNA sense
ggagatcgtgaaccaggacaa

SEQ ID NO: 92 - DNA, 21 bp, Homo sapiens
  misc_feature (1)..(21): 5′ sense dopamine-beta-hydroxylase target protein 5′ DNA sense
aaccaggacaatcactacagc

SEQ ID NO: 93 - DNA, 21 bp, Homo sapiens
  misc_feature (1)..(21): 5′ sense dopamine-beta-hydroxylase target protein 5′ DNA sense
aatcactacagccctcacttc

SEQ ID NO: 94 - DNA, 25 bp, Homo sapiens
  misc_feature (1)..(25): 5′ sense dopamine-beta-hydroxylase target protein 5′ DNA sense
acttccaggagatccgcatgttgaa

SEQ ID NO: 95 - DNA, 23 bp, Homo sapiens
  misc_feature (1)..(23): 5′ sense dopamine-beta-hydroxylase target protein 5′ DNA sense
ttccaggagatccgcatgttgaa

SEQ ID NO: 96 - DNA, 21 bp, Homo sapiens
  misc_feature (1)..(21): 5′ sense dopamine-beta-hydroxylase target protein 5′ DNA sense
ccaggagatccgcatgttgaa

SEQ ID NO: 97 - DNA, 19 bp, Homo sapiens
  misc_feature (1)..(19): 5′ sense dopamine-beta-hydroxylase target protein 5′ DNA sense
aggagatccgcatgttgaa

SEQ ID NO: 98 - DNA, 21 bp, Homo sapiens
  misc_feature (1)..(21): 5′ sense dopamine-beta-hydroxylase target protein 5′ DNA sense
ggagatccgcatgttgaagaa

SEQ ID NO: 99 - DNA, 21 bp, Homo sapiens
  misc_feature (1)..(21): 5′ sense dopamine-beta-hydroxylase target protein 5′ DNA sense
aagaaggtcgtgtcggtccat

SEQ ID NO: 100 - DNA, 21 bp, Homo sapiens
  misc_feature (1)..(21): 5′ sense dopamine-beta-hydroxylase target protein 5′ DNA sense
aaggtcgtgtcggtccatccg

SEQ ID NO: 101 - DNA, 21 bp, Homo sapiens
  misc_feature (1)..(21): 5′ sense dopamine-beta-hydroxylase target protein 5′ DNA sense
catcacctcctgcacgtacaa

SEQ ID NO: 102 - DNA, 21 bp, Homo sapiens
  misc_feature (1)..(21): 5′ sense dopamine-beta-hydroxylase target protein 5′ DNA sense
tcctgcacgtacaacacagaa

SEQ ID NO: 103 - DNA, 21 bp, Homo sapiens
  misc_feature (1)..(21): 5′ sense dopamine-beta-hydroxylase target protein 5′ DNA sense
aacacagaagaccgggagctg

SEQ ID NO: 104 - DNA, 21 bp, Homo sapiens
  misc_feature (1)..(21): 5′ sense dopamine-beta-hydroxylase target protein 5′ DNA sense
aagaccgggagctggccacag

SEQ ID NO: 105 - DNA, 21 bp, Homo sapiens
  misc_feature (1)..(21): 5′ sense dopamine-beta-hydroxylase target protein 5′ DNA sense
cctggaggagatgtgtgtcaa

SEQ ID NO: 106 - DNA, 21 bp, Homo sapiens
  misc_feature (1)..(21): 5′ sense dopamine-beta-hydroxylase target protein 5′ DNA sense
aactacgtgcactactacccc

SEQ ID NO: 107 - DNA, 21 bp, Homo sapiens
  misc_feature (1)..(21): 5′ sense dopamine-beta-hydroxylase target protein 5′ DNA sense
gacgcagctggagctctgcaa

SEQ ID NO: 108 - DNA, 21 bp, Homo sapiens
  misc_feature (1)..(21): 5′ sense dopamine-beta-hydroxylase target protein 5′ DNA sense
aagagcgctgtggacgccggc

SEQ ID NO: 109 - DNA, 21 bp, Homo sapiens
  misc_feature (1)..(21): 5′ sense dopamine-beta-hydroxylase target protein 5′ DNA sense
ggacgccggcttcctgcagaa

SEQ ID NO: 110 - DNA, 21 bp, Homo sapiens
  misc_feature (1)..(21): 5′ sense dopamine-beta-hydroxylase target protein 5′ DNA sense
gaagtacttccacctcatcaa

SEQ ID NO: 111 - DNA, 21 bp, Homo sapiens
  misc_feature (1)..(21): 5′ sense dopamine-beta-hydroxylase target protein 5′ DNA sense
aagtacttccacctcatcaac

SEQ ID NO: 112 - DNA, 21 bp, Homo sapiens
  misc_feature (1)..(21): 5′ sense dopamine-beta-hydroxylase target protein 5′ DNA sense
ccacctcatcaacaggttcaa

SEQ ID NO: 113 - DNA, 21 bp, Homo sapiens
  misc_feature (1)..(21): 5′ sense dopamine-beta-hydroxylase target protein 5′ DNA sense
cctcatcaacaggttcaacaa

SEQ ID NO: 114 - DNA, 21 bp, Homo sapiens
  misc_feature (1)..(21): 5′ sense dopamine-beta-hydroxylase target protein 5′ DNA sense
aacaggttcaacaacgaggat

SEQ ID NO: 115 - DNA, 21 bp, Homo sapiens
  misc_feature (1)..(21): 5′ sense dopamine-beta-hydroxylase target protein 5′ DNA sense
aacaacgaggatgtctgcacc

SEQ ID NO: 116 - DNA, 21 bp, Homo sapiens
  misc_feature (1)..(21): 5′ sense dopamine-beta-hydroxylase target protein 5′ DNA sense
aacgaggatgtctgcacctgc

SEQ ID NO: 117 - DNA, 21 bp, Homo sapiens
  misc_feature (1)..(21): 5′ sense dopamine-beta-hydroxylase target protein 5′ DNA sense
gttcacctctgttccctggaa

SEQ ID NO: 118 - DNA, 21 bp, Homo sapiens
  misc_feature (1)..(21): 5′ sense dopamine-beta-hydroxylase target protein 5′ DNA sense
tgttccctggaactccttcaa

SEQ ID NO: 119 - DNA, 21 bp, Homo sapiens
  misc_feature (1)..(21): 5′ sense dopamine-beta-hydroxylase target protein 5′ DNA sense
aactccttcaaccgcgacgta

SEQ ID NO: 120 - DNA, 21 bp, Homo sapiens
  misc_feature (1)..(21): 5′ sense dopamine-beta-hydroxylase target protein 5′ DNA sense
cttcaaccgcgacgtactgaa

SEQ ID NO: 121 - DNA, 21 bp, Homo sapiens
  misc_feature (1)..(21): 5′ sense dopamine-beta-hydroxylase target protein 5′ DNA sense
aaccgcgacgtactgaaggcc

SEQ ID NO: 122 - DNA, 21 bp, Homo sapiens
  misc_feature (1)..(21): 5′ sense dopamine-beta-hydroxylase target protein 5′ DNA sense
aaggccctgtacagcttcgcg

SEQ ID NO: 123 - DNA, 21 bp, Homo sapiens
  misc_feature (1)..(21): 5′ sense dopamine-beta-hydroxylase target protein 5′ DNA sense
gcccatctccatgcactgcaa

SEQ ID NO: 124 - DNA, 21 bp, Homo sapiens
  misc_feature (1)..(21): 5′ sense dopamine-beta-hydroxylase target protein 5′ DNA sense
catctccatgcactgcaacaa

SEQ ID NO: 125 - DNA, 21 bp, Homo sapiens
  misc_feature (1)..(21): 5′ sense dopamine-beta-hydroxylase target protein 5′ DNA sense
aacaagtcctcagccgtccgc

SEQ ID NO: 126 - DNA, 21 bp, Homo sapiens
  misc_feature (1)..(21): 5′ sense dopamine-beta-hydroxylase target protein 5′ DNA sense
aagtcctcagccgtccgcttc

SEQ ID NO: 127 - DNA, 21 bp, Homo sapiens
  misc_feature (1)..(21): 5′ sense dopamine-beta-hydroxylase target protein 5′ DNA sense
gccgtccgcttccagggtgaa

SEQ ID NO: 128 - DNA, 21 bp, Homo sapiens
  misc_feature (1)..(21): 5′ sense dopamine-beta-hydroxylase target protein 5′ DNA sense
ccgcttccagggtgaatggaa

SEQ ID NO: 129 - DNA, 21 bp, Homo sapiens
  misc_feature (1)..(21): 5′ sense dopamine-beta-hydroxylase target protein 5′ DNA sense
aatggaacctgcagcccctgc

SEQ ID NO: 130 - DNA, 21 bp, Homo sapiens
  misc_feature (1)..(21): 5′ sense dopamine-beta-hydroxylase target protein 5′ DNA sense
gaacctgcagcccctgcccaa

SEQ ID NO: 131 - DNA, 21 bp, Homo sapiens
  misc_feature (1)..(21): 5′ sense dopamine-beta-hydroxylase target protein 5′ DNA sense
aacctgcagcccctgcccaag

SEQ ID NO: 132 - DNA, 21 bp, Homo sapiens
  misc_feature (1)..(21): 5′ sense dopamine-beta-hydroxylase target protein 5′ DNA sense
aaggtcatctccacactggaa

SEQ ID NO: 133 - DNA, 21 bp, Homo sapiens
  misc_feature (1)..(21): 5′ sense dopamine-beta-hydroxylase target protein 5′ DNA sense
aagagcccaccccacagtgcc

SEQ ID NO: 134 - DNA, 21 bp, Homo sapiens
  misc_feature (1)..(21): 5′ sense dopamine-beta-hydroxylase target protein 5′ DNA sense
gccccaccagccagggccgaa

SEQ ID NO: 135 - DNA, 21 bp, Homo sapiens
  misc_feature (1)..(21): 5′ sense dopamine-beta-hydroxylase target protein 5′ DNA sense
aagccctgctggccccaccgt

SEQ ID NO: 136 - DNA, 21 bp, Homo sapiens
  misc_feature (1)..(21): 5′ sense dopamine-beta-hydroxylase target protein 5′ DNA sense
tgtcagcattggtgggggcaa

SEQ ID NO: 137 - DNA, 21 bp, Homo sapiens
  misc_feature (1)..(21): 5′ sense dopamine-beta-hydroxylase target protein 5′ DNA sense
gtcagcattggtgggggcaaa

SEQ ID NO: 138 - RNA, 21 nt, Artificial Sequence
  siRNA sequences of dopamine-beta-hydroxylase 5′ RNA sense
gucccuggagcucucauggaa

SEQ ID NO: 139 - RNA, 21 nt, Artificial Sequence
  siRNA sequences of dopamine-beta-hydroxylase 5′ RNA sense
aaugucagcuacacccaggag

SEQ ID NO: 140 - RNA, 21 nt, Artificial Sequence
  siRNA sequences of dopamine-beta-hydroxylase 5′ RNA sense
gcuccuggugcggaggcucaa

SEQ ID NO: 141 - RNA, 21 nt, Artificial Sequence
  siRNA sequences of dopamine-beta-hydroxylase 5′ RNA sense
aaggcuggcguccuguuuggg

SEQ ID NO: 142 - RNA, 25 nt, Artificial Sequence
  siRNA sequences of dopamine-beta-hydroxylase 5′ RNA sense
uguccgaccguggcgagcuugagaa

SEQ ID NO: 143 - RNA, 23 nt, Artificial Sequence
  siRNA sequences of dopamine-beta-hydroxylase 5′ RNA sense
uccgaccguggcgagcuugagaa

SEQ ID NO: 144 - RNA, 21 nt, Artificial Sequence
  siRNA sequences of dopamine-beta-hydroxylase 5′ RNA sense
cgaccguggcgagcuugagaa

SEQ ID NO: 145 - RNA, 19 nt, Artificial Sequence
  siRNA sequences of dopamine-beta-hydroxylase 5′ RNA sense
accguggcgagcuugagaa

SEQ ID NO: 146 - RNA, 21 nt, Artificial Sequence
  siRNA sequences of dopamine-beta-hydroxylase 5′ RNA sense
aacgcagaucucguggugcuc

SEQ ID NO: 147 - RNA, 21 nt, Artificial Sequence
  siRNA sequences of dopamine-beta-hydroxylase 5′ RNA sense
ggacgccuggagugaccagaa

SEQ ID NO: 148 - RNA, 21 nt, Artificial Sequence
  siRNA sequences of dopamine-beta-hydroxylase 5′ RNA sense
aaggggcagauccaccuggau

SEQ ID NO: 149 - RNA, 21 nt, Artificial Sequence
  siRNA sequences of dopamine-beta-hydroxylase 5′ RNA sense
caggugcagaggaccccagaa

SEQ ID NO: 150 - RNA, 21 nt, Artificial Sequence
  siRNA sequences of dopamine-beta-hydroxylase 5′ RNA sense
aaggccugacccugcuuuuca

SEQ ID NO: 151 - RNA, 19 nt, Artificial Sequence
  siRNA sequences of dopamine-beta-hydroxylase 5′ RNA sense
aaggccugacccugcuuuu

SEQ ID NO: 152 - RNA, 23 nt, Artificial Sequence
  siRNA sequences of dopamine-beta-hydroxylase 5′ RNA sense
aaggccugacccugcuuuucaag

SEQ ID NO: 153 - RNA, 25 nt, Artificial Sequence
  siRNA sequences of dopamine-beta-hydroxylase 5′ RNA sense
aaggccugacccugcuuuucaagag

SEQ ID NO: 154 - RNA, 21 nt, Artificial Sequence
  siRNA sequences of dopamine-beta-hydroxylase 5′ RNA sense
aggccugacccugcuuuucaa

SEQ ID NO: 155 - RNA, 21 nt, Artificial Sequence
  siRNA sequences of dopamine-beta-hydroxylase 5′ RNA sense
aagaggcccuuuggcaccugc

SEQ ID NO: 156 - RNA, 21 nt, Artificial Sequence
  siRNA sequences of dopamine-beta-hydroxylase 5′ RNA sense
cuuuggcaccugcgaccccaa

SEQ ID NO: 157 - RNA, 21 nt, Artificial Sequence
  siRNA sequences of dopamine-beta-hydroxylase 5′ RNA sense
cccaaggauuaccucauugaa

SEQ ID NO: 158 - RNA, 21 nt, Artificial Sequence
  siRNA sequences of dopamine-beta-hydroxylase 5′ RNA sense
aaggauuaccucauugaagac

SEQ ID NO: 159 - RNA, 21 nt, Artificial Sequence
  siRNA sequences of dopamine-beta-hydroxylase 5′ RNA sense
aagacggcacuguccacuugg

SEQ ID NO: 160 - RNA, 21 nt, Artificial Sequence
  siRNA sequences of dopamine-beta-hydroxylase 5′ RNA sense
ccggucacuggaggccaucaa

SEQ ID NO: 161 - RNA, 21 nt, Artificial Sequence
  siRNA sequences of dopamine-beta-hydroxylase 5′ RNA sense
aacggcucgggccugcagaug

SEQ ID NO: 162 - RNA, 21 nt, Artificial Sequence
  siRNA sequences of dopamine-beta-hydroxylase 5′ RNA sense
gcagagggugcagcuccugaa

SEQ ID NO: 163 - RNA, 21 nt, Artificial Sequence
  siRNA sequences of dopamine-beta-hydroxylase 5′ RNA sense
ggugcagcuccugaagcccaa

SEQ ID NO: 164 - RNA, 21 nt, Artificial Sequence
  siRNA sequences of dopamine-beta-hydroxylase 5′ RNA sense
cugaagcccaauauccccgaa

SEQ ID NO: 165 - RNA, 21 nt, Artificial Sequence
  siRNA sequences of dopamine-beta-hydroxylase 5′ RNA sense
aagcccaauauccccgaaccg

SEQ ID NO: 166 - RNA, 21 nt, Artificial Sequence
  siRNA sequences of dopamine-beta-hydroxylase 5′ RNA sense
aauauccccgaaccggaguug

SEQ ID NO: 167 - RNA, 21 nt, Artificial Sequence
  siRNA sequences of dopamine-beta-hydroxylase 5′ RNA sense
aaccggaguugcccucagacg

SEQ ID NO: 168 - RNA, 21 nt, Artificial Sequence
  siRNA sequences of dopamine-beta-hydroxylase 5′ RNA sense
gcgugcaccauggagguccaa

SEQ ID NO: 169 - RNA, 21 nt, Artificial Sequence
  siRNA sequences of dopamine-beta-hydroxylase 5′ RNA sense
cauggagguccaagcucccaa

SEQ ID NO: 170 - RNA, 21 nt, Artificial Sequence
  siRNA sequences of dopamine-beta-hydroxylase 5′ RNA sense
aagcucccaauauccagaucc

SEQ ID NO: 171 - RNA, 21 nt, Artificial Sequence
  siRNA sequences of dopamine-beta-hydroxylase 5′ RNA sense
aauauccagauccccagccag

SEQ ID NO: 172 - RNA, 25 nt, Artificial Sequence
  siRNA sequences of dopamine-beta-hydroxylase 5′ RNA sense
agaccacguacuggugcuacauuaa

SEQ ID NO: 173 - RNA, 23 nt, Artificial Sequence
  siRNA sequences of dopamine-beta-hydroxylase 5′ RNA sense
accacguacuggugcuacauuaa

SEQ ID NO: 174 - RNA, 21 nt, Artificial Sequence
  siRNA sequences of dopamine-beta-hydroxylase 5′ RNA sense
cacguacuggugcuacauuaa

SEQ ID NO: 175 - RNA, 19 nt, Artificial Sequence
  siRNA sequences of dopamine-beta-hydroxylase 5′ RNA sense
cguacuggugcuacauuaa

SEQ ID NO: 176 - RNA, 21 nt, Artificial Sequence
  siRNA sequences of dopamine-beta-hydroxylase 5′ RNA sense
gcuacauuaaggagcuuccaa

SEQ ID NO: 177 - RNA, 21 nt, Artificial Sequence
  siRNA sequences of dopamine-beta-hydroxylase 5′ RNA sense
cuacauuaaggagcuuccaaa

SEQ ID NO: 178 - RNA, 21 nt, Artificial Sequence
  siRNA sequences of dopamine-beta-hydroxylase 5′ RNA sense
aaggagcuuccaaagggcuuc

SEQ ID NO: 179 - RNA, 21 nt, Artificial Sequence
  siRNA sequences of dopamine-beta-hydroxylase 5′ RNA sense
aaagggcuucucucggcacca

SEQ ID NO: 180 - RNA, 21 nt, Artificial Sequence
  siRNA sequences of dopamine-beta-hydroxylase 5′ RNA sense
aagggcuucucucggcaccac

SEQ ID NO: 181 - RNA, 25 nt, Artificial Sequence
  siRNA sequences of dopamine-beta-hydroxylase 5′ RNA sense
gcuucucucggcaccacauuaucaa

SEQ ID NO: 182 - RNA, 23 nt, Artificial Sequence
  siRNA sequences of dopamine-beta-hydroxylase 5′ RNA sense
uucucucggcaccacauuaucaa

SEQ ID NO: 183 - RNA, 21 nt, Artificial Sequence
  siRNA sequences of dopamine-beta-hydroxylase 5′ RNA sense
cucucggcaccacauuaucaa

SEQ ID NO: 184 - RNA, 19 nt, Artificial Sequence
  siRNA sequences of dopamine-beta-hydroxylase 5′ RNA sense
cucggcaccacauuaucaa

SEQ ID NO: 185 - RNA, 21 nt, Artificial Sequence
  siRNA sequences of dopamine-beta-hydroxylase 5′ RNA sense
aaguacgagcccaucgucacc

SEQ ID NO: 186 - RNA, 21 nt, Artificial Sequence
  siRNA sequences of dopamine-beta-hydroxylase 5′ RNA sense
guacgagcccaucgucaccaa

SEQ ID NO: 187 - RNA, 21 nt, Artificial Sequence
  siRNA sequences of dopamine-beta-hydroxylase 5′ RNA sense
gcccaucgucaccaagggcaa

SEQ ID NO: 188 - RNA, 21 nt, Artificial Sequence
  siRNA sequences of dopamine-beta-hydroxylase 5′ RNA sense
aagggcaaugaggcccuuguc

SEQ ID NO: 189 - RNA, 21 nt, Artificial Sequence
  siRNA sequences of dopamine-beta-hydroxylase 5′ RNA sense
aaugaggcccuuguccaccac

SEQ ID NO: 190 - RNA, 21 nt, Artificial Sequence
  siRNA sequences of dopamine-beta-hydroxylase 5′ RNA sense
gcccuuguccaccacauggaa

SEQ ID NO: 191 - RNA, 21 nt, Artificial Sequence
  siRNA sequences of dopamine-beta-hydroxylase 5′ RNA sense
aagucuuccagugcgcccccg

SEQ ID NO: 192 - RNA, 21 nt, Artificial Sequence
  siRNA sequences of dopamine-beta-hydroxylase 5′ RNA sense
cagcgggcccugcgacuccaa

SEQ ID NO: 193 - RNA, 25 nt, Artificial Sequence
  siRNA sequences of dopamine-beta-hydroxylase 5′ RNA sense
cgggcccugcgacuccaagaugaaa

SEQ ID NO: 194 - RNA, 23 nt, Artificial Sequence
  siRNA sequences of dopamine-beta-hydroxylase 5′ RNA sense
ggcccugcgacuccaagaugaaa

SEQ ID NO: 195 - RNA, 21 nt, Artificial Sequence
  siRNA sequences of dopamine-beta-hydroxylase 5′ RNA sense
gcccugcgacuccaagaugaa

SEQ ID NO: 196 - RNA, 21 nt, Artificial Sequence
  siRNA sequences of dopamine-beta-hydroxylase 5′ RNA sense
cccugcgacuccaagaugaaa

SEQ ID NO: 197 - RNA, 19 nt, Artificial Sequence
  siRNA sequences of dopamine-beta-hydroxylase 5′ RNA sense
cugcgacuccaagaugaaa

SEQ ID NO: 198 - RNA, 21 nt, Artificial Sequence
  siRNA sequences of dopamine-beta-hydroxylase 5′ RNA sense
aagaugaaacccgaccgccuc

SEQ ID NO: 199 - RNA, 21 nt, Artificial Sequence
  siRNA sequences of dopamine-beta-hydroxylase 5′ RNA sense
gaugaaacccgaccgccucaa

SEQ ID NO: 200 - RNA, 21 nt, Artificial Sequence
  siRNA sequences of dopamine-beta-hydroxylase 5′ RNA sense
aaacccgaccgccucaacuac

SEQ ID NO: 201 - RNA, 21 nt, Artificial Sequence
  siRNA sequences of dopamine-beta-hydroxylase 5′ RNA sense
aacccgaccgccucaacuacu

SEQ ID NO: 202 - RNA, 21 nt, Artificial Sequence
  siRNA sequences of dopamine-beta-hydroxylase 5′ RNA sense
aacuacugccgccacgugcug

SEQ ID NO: 203 - RNA, 21 nt, Artificial Sequence
  siRNA sequences of dopamine-beta-hydroxylase 5′ RNA sense
cgccugggcccugggugccaa

SEQ ID NO: 204 - RNA, 21 nt, Artificial Sequence
  siRNA sequences of dopamine-beta-hydroxylase 5′ RNA sense
aaggcauuuuacuacccagag

SEQ ID NO: 205 - RNA, 21 nt, Artificial Sequence
  siRNA sequences of dopamine-beta-hydroxylase 5′ RNA sense
gcauuuuacuacccagaggaa

SEQ ID NO: 206 - RNA, 21 nt, Artificial Sequence
  siRNA sequences of dopamine-beta-hydroxylase 5′ RNA sense
aagccggccuugccuucgggg

SEQ ID NO: 207 - RNA, 21 nt, Artificial Sequence
  siRNA sequences of dopamine-beta-hydroxylase 5′ RNA sense
uccagauaucuccgccuggaa

SEQ ID NO: 208 - RNA, 21 nt, Artificial Sequence
  siRNA sequences of dopamine-beta-hydroxylase 5′ RNA sense
ccuggaaguucacuaccacaa

SEQ ID NO: 209 - RNA, 21 nt, Artificial Sequence
  siRNA sequences of dopamine-beta-hydroxylase 5′ RNA sense
aaguucacuaccacaacccac

SEQ ID NO: 210 - RNA, 21 nt, Artificial Sequence
  siRNA sequences of dopamine-beta-hydroxylase 5′ RNA sense
cacaacccacuggugauagaa

SEQ ID NO: 211 - RNA, 21 nt, Artificial Sequence
  siRNA sequences of dopamine-beta-hydroxylase 5′ RNA sense
aacccacuggugauagaagga

SEQ ID NO: 212 - RNA, 21 nt, Artificial Sequence
  siRNA sequences of dopamine-beta-hydroxylase 5′ RNA sense
cacuggugauagaaggacgaa

SEQ ID NO: 213 - RNA, 21 nt, Artificial Sequence
  siRNA sequences of dopamine-beta-hydroxylase 5′ RNA sense
acuggugauagaaggacgaaa

SEQ ID NO: 214 - RNA, 21 nt, Artificial Sequence
  siRNA sequences of dopamine-beta-hydroxylase 5′ RNA sense
aaggacgaaacgacuccucag

SEQ ID NO: 215 - RNA, 21 nt, Artificial Sequence
  siRNA sequences of dopamine-beta-hydroxylase 5′ RNA sense
aaacgacuccucaggcauccg

SEQ ID NO: 216 - RNA, 21 nt, Artificial Sequence
  siRNA sequences of dopamine-beta-hydroxylase 5′ RNA sense
aacgacuccucaggcauccgc

SEQ ID NO: 217 - RNA, 21 nt, Artificial Sequence
  siRNA sequences of dopamine-beta-hydroxylase 5′ RNA sense
ccgcuuguacuacacagccaa

SEQ ID NO: 218 - RNA, 21 nt, Artificial Sequence
  siRNA sequences of dopamine-beta-hydroxylase 5′ RNA sense
agccaagcugcggcgcuucaa

SEQ ID NO: 219 - RNA, 21 nt, Artificial Sequence
  siRNA sequences of dopamine-beta-hydroxylase 5′ RNA sense
aagcugcggcgcuucaacgcg

SEQ ID NO: 220 - RNA, 21 nt, Artificial Sequence
  siRNA sequences of dopamine-beta-hydroxylase 5′ RNA sense
aacgcggggaucauggagcug

SEQ ID NO: 221 - RNA, 21 nt, Artificial Sequence
  siRNA sequences of dopamine-beta-hydroxylase 5′ RNA sense
cacuggcuacugcacggacaa

SEQ ID NO: 222 - RNA, 21 nt, Artificial Sequence
  siRNA sequences of dopamine-beta-hydroxylase 5′ RNA sense
aagugcacccagcuggcacug

SEQ ID NO: 223 - RNA, 21 nt, Artificial Sequence
  siRNA sequences of dopamine-beta-hydroxylase 5′ RNA sense
acacacaccugacugggagaa

SEQ ID NO: 224 - RNA, 21 nt, Artificial Sequence
  siRNA sequences of dopamine-beta-hydroxylase 5′ RNA sense
cacacaccugacugggagaaa

SEQ ID NO: 225 - RNA, 21 nt, Artificial Sequence
  siRNA sequences of dopamine-beta-hydroxylase 5′ RNA sense
aaagguggucacagugcuggu

SEQ ID NO: 226 - RNA, 21 nt, Artificial Sequence
  siRNA sequences of dopamine-beta-hydroxylase 5′ RNA sense
aagguggucacagugcugguc

SEQ ID NO: 227 - RNA, 21 nt, Artificial Sequence
  siRNA sequences of dopamine-beta-hydroxylase 5′ RNA sense
ccgggagugggagaucgugaa

SEQ ID NO: 228 - RNA, 21 nt, Artificial Sequence
  siRNA sequences of dopamine-beta-hydroxylase 5′ RNA sense
ggagaucgugaaccaggacaa

SEQ ID NO: 229 - RNA, 21 nt, Artificial Sequence
  siRNA sequences of dopamine-beta-hydroxylase 5′ RNA sense
aaccaggacaaucacuacagc

SEQ ID NO: 230 - RNA, 21 nt, Artificial Sequence
  siRNA sequences of dopamine-beta-hydroxylase 5′ RNA sense
aaucacuacagcccucacuuc

SEQ ID NO: 231 - RNA, 25 nt, Artificial Sequence
  siRNA sequences of dopamine-beta-hydroxylase 5′ RNA sense
acuuccaggagauccgcauguugaa

SEQ ID NO: 232 - RNA, 23 nt, Artificial Sequence
  siRNA sequences of dopamine-beta-hydroxylase 5′ RNA sense
uuccaggagauccgcauguugaa

SEQ ID NO: 233 - RNA, 21 nt, Artificial Sequence
  siRNA sequences of dopamine-beta-hydroxylase 5′ RNA sense
ccaggagauccgcauguugaa

SEQ ID NO: 234 - RNA, 19 nt, Artificial Sequence
  siRNA sequences of dopamine-beta-hydroxylase 5′ RNA sense
aggagauccgcauguugaa

SEQ ID NO: 235 - RNA, 21 nt, Artificial Sequence
  siRNA sequences of dopamine-beta-hydroxylase 5′ RNA sense
ggagauccgcauguugaagaa

SEQ ID NO: 236 - RNA, 21 nt, Artificial Sequence
  siRNA sequences of dopamine-beta-hydroxylase 5′ RNA sense
aagaaggucgugucgguccau

SEQ ID NO: 237 - RNA, 21 nt, Artificial Sequence
  siRNA sequences of dopamine-beta-hydroxylase 5′ RNA sense
aaggucgugucgguccauccg

SEQ ID NO: 238 - RNA, 21 nt, Artificial Sequence
  siRNA sequences of dopamine-beta-hydroxylase 5′ RNA sense
caucaccuccugcacguacaa

SEQ ID NO: 239 - RNA, 21 nt, Artificial Sequence
  siRNA sequences of dopamine-beta-hydroxylase 5′ RNA sense
uccugcacguacaacacagaa

SEQ ID NO: 240 - RNA, 21 nt, Artificial Sequence
  siRNA sequences of dopamine-beta-hydroxylase 5′ RNA sense
aacacagaagaccgggagcug

SEQ ID NO: 241 - RNA, 21 nt, Artificial Sequence
  siRNA sequences of dopamine-beta-hydroxylase 5′ RNA sense
aagaccgggagcuggccacag

SEQ ID NO: 242 - RNA, 21 nt, Artificial Sequence
  siRNA sequences of dopamine-beta-hydroxylase 5′ RNA sense
ccuggaggagaugugugucaa

SEQ ID NO: 243 - RNA, 21 nt, Artificial Sequence
  siRNA sequences of dopamine-beta-hydroxylase 5′ RNA sense
aacuacgugcacuacuacccc

SEQ ID NO: 244 - RNA, 21 nt, Artificial Sequence
  siRNA sequences of dopamine-beta-hydroxylase 5′ RNA sense
gacgcagcuggagcucugcaa

SEQ ID NO: 245 - RNA, 21 nt, Artificial Sequence
  siRNA sequences of dopamine-beta-hydroxylase 5′ RNA sense
aagagcgcuguggacgccggc

SEQ ID NO: 246 - RNA, 21 nt, Artificial Sequence
  siRNA sequences of dopamine-beta-hydroxylase 5′ RNA sense
ggacgccggcuuccugcagaa

SEQ ID NO: 247 - RNA, 21 nt, Artificial Sequence
  siRNA sequences of dopamine-beta-hydroxylase 5′ RNA sense
gaaguacuuccaccucaucaa

SEQ ID NO: 248 - RNA, 21 nt, Artificial Sequence
  siRNA sequences of dopamine-beta-hydroxylase 5′ RNA sense
aaguacuuccaccucaucaac

SEQ ID NO: 249 - RNA, 21 nt, Artificial Sequence
  siRNA sequences of dopamine-beta-hydroxylase 5′ RNA sense
ccaccucaucaacagguucaa

SEQ ID NO: 250 - RNA, 21 nt, Artificial Sequence
  siRNA sequences of dopamine-beta-hydroxylase 5′ RNA sense
ccucaucaacagguucaacaa

SEQ ID NO: 251 - RNA, 21 nt, Artificial Sequence
  siRNA sequences of dopamine-beta-hydroxylase 5′ RNA sense
aacagguucaacaacgaggau

SEQ ID NO: 252 - RNA, 21 nt, Artificial Sequence
  siRNA sequences of dopamine-beta-hydroxylase 5′ RNA sense
aacaacgaggaugucugcacc

SEQ ID NO: 253 - RNA, 21 nt, Artificial Sequence
  siRNA sequences of dopamine-beta-hydroxylase 5′ RNA sense
aacgaggaugucugcaccugc

SEQ ID NO: 254 - RNA, 21 nt, Artificial Sequence
  siRNA sequences of dopamine-beta-hydroxylase 5′ RNA sense
guucaccucuguucccuggaa

SEQ ID NO: 255 - RNA, 21 nt, Artificial Sequence
  siRNA sequences of dopamine-beta-hydroxylase 5′ RNA sense
uguucccuggaacuccuucaa

SEQ ID NO: 256 - RNA, 21 nt, Artificial Sequence
  siRNA sequences of dopamine-beta-hydroxylase 5′ RNA sense
aacuccuucaaccgcgacgua

SEQ ID NO: 257 - RNA, 21 nt, Artificial Sequence
  siRNA sequences of dopamine-beta-hydroxylase 5′ RNA sense
cuucaaccgcgacguacugaa

SEQ ID NO: 258 - RNA, 21 nt, Artificial Sequence
  siRNA sequences of dopamine-beta-hydroxylase 5′ RNA sense
aaccgcgacguacugaaggcc

SEQ ID NO: 259 - RNA, 21 nt, Artificial Sequence
  siRNA sequences of dopamine-beta-hydroxylase 5′ RNA sense
aaggcccuguacagcuucgcg

SEQ ID NO: 260 - RNA, 21 nt, Artificial Sequence
  siRNA sequences of dopamine-beta-hydroxylase 5′ RNA sense
gcccaucuccaugcacugcaa

SEQ ID NO: 261 - RNA, 21 nt, Artificial Sequence
  siRNA sequences of dopamine-beta-hydroxylase 5′ RNA sense
caucuccaugcacugcaacaa

SEQ ID NO: 262 - RNA, 21 nt, Artificial Sequence
  siRNA sequences of dopamine-beta-hydroxylase 5′ RNA sense
aacaaguccucagccguccgc

SEQ ID NO: 263 - RNA, 21 nt, Artificial Sequence
  siRNA sequences of dopamine-beta-hydroxylase 5′ RNA sense
aaguccucagccguccgcuuc

SEQ ID NO: 264 - RNA, 21 nt, Artificial Sequence
  siRNA sequences of dopamine-beta-hydroxylase 5′ RNA sense
gccguccgcuuccagggugaa

SEQ ID NO: 265 - RNA, 21 nt, Artificial Sequence
  siRNA sequences of dopamine-beta-hydroxylase 5′ RNA sense
ccgcuuccagggugaauggaa

SEQ ID NO: 266 - RNA, 21 nt, Artificial Sequence
  siRNA sequences of dopamine-beta-hydroxylase 5′ RNA sense
aauggaaccugcagccccugc

SEQ ID NO: 267 - RNA, 21 nt, Artificial Sequence
  siRNA sequences of dopamine-beta-hydroxylase 5′ RNA sense
gaaccugcagccccugcccaa

SEQ ID NO: 268 - RNA, 21 nt, Artificial Sequence
  siRNA sequences of dopamine-beta-hydroxylase 5′ RNA sense
aaccugcagccccugcccaag

SEQ ID NO: 269 - RNA, 21 nt, Artificial Sequence
  siRNA sequences of dopamine-beta-hydroxylase 5′ RNA sense
aaggucaucuccacacuggaa

SEQ ID NO: 270 - RNA, 21 nt, Artificial Sequence
  siRNA sequences of dopamine-beta-hydroxylase 5′ RNA sense
aagagcccaccccacagugcc

SEQ ID NO: 271 - RNA, 21 nt, Artificial Sequence
  siRNA sequences of dopamine-beta-hydroxylase 5′ RNA sense
gccccaccagccagggccgaa

SEQ ID NO: 272 - RNA, 21 nt, Artificial Sequence
  siRNA sequences of dopamine-beta-hydroxylase 5′ RNA sense
aagcccugcuggccccaccgu

SEQ ID NO: 273 - RNA, 21 nt, Artificial Sequence
  siRNA sequences of dopamine-beta-hydroxylase 5′ RNA sense
ugucagcauuggugggggcaa

SEQ ID NO: 274 - RNA, 21 nt, Artificial Sequence
  siRNA sequences of dopamine-beta-hydroxylase 5′ RNA sense
gucagcauuggugggggcaaa

SEQ ID NO: 275 - RNA, 21 nt, Artificial Sequence
  siRNA sequences of dopamine-beta-hydroxylase 5 RNA antisense
uuccaugagagcuccagggac

SEQ ID NO: 276 - RNA, 21 nt, Artificial Sequence
  siRNA sequences of dopamine-beta-hydroxylase 6 RNA antisense
cuccuggguguagcugacauu

SEQ ID NO: 277 - RNA, 21 nt, Artificial Sequence
  siRNA sequences of dopamine-beta-hydroxylase 7 RNA antisense
uugagccuccgcaccaggagc

SEQ ID NO: 278 - RNA, 21 nt, Artificial Sequence
  siRNA sequences of dopamine-beta-hydroxylase 8 RNA antisense
cccaaacaggacgccagccuu

SEQ ID NO: 279 - RNA, 25 nt, Artificial Sequence
  siRNA sequences of dopamine-beta-hydroxylase 9 RNA antisense
uucucaagcucgccacggucggaca

SEQ ID NO: 280 - RNA, 23 nt, Artificial Sequence
  siRNA sequences of dopamine-beta-hydroxylase 10 RNA antisense
uucucaagcucgccacggucgga

SEQ ID NO: 281 - RNA, 21 nt, Artificial Sequence
  siRNA sequences of dopamine-beta-hydroxylase 11 RNA antisense
uucucaagcucgccacggucg

SEQ ID NO: 282 - RNA, 19 nt, Artificial Sequence
  siRNA sequences of dopamine-beta-hydroxylase 12 RNA antisense
uucucaagcucgccacggu

SEQ ID NO: 283 - RNA, 21 nt, Artificial Sequence
  siRNA sequences of dopamine-beta-hydroxylase 13 RNA antisense
gagcaccacgagaucugcguu

SEQ ID NO: 284 - RNA, 21 nt, Artificial Sequence
  siRNA sequences of dopamine-beta-hydroxylase 14 RNA antisense
uucuggucacuccaggcgucc

SEQ ID NO: 285 - RNA, 21 nt, Artificial Sequence
  siRNA sequences of dopamine-beta-hydroxylase 15 RNA antisense
auccagguggaucugccccuu

SEQ ID NO: 286 - RNA, 21 nt, Artificial Sequence
  siRNA sequences of dopamine-beta-hydroxylase 16 RNA antisense
uucugggguccucugcaccug

SEQ ID NO: 287 - RNA, 21 nt, Artificial Sequence
  siRNA sequences of dopamine-beta-hydroxylase 17 RNA antisense
ugaaaagcagggucaggccuu

SEQ ID NO: 288 - RNA, 19 nt, Artificial Sequence
  siRNA sequences of dopamine-beta-hydroxylase 18 RNA antisense
aaaagcagggucaggccuu

SEQ ID NO: 289 - RNA, 23 nt, Artificial Sequence
  siRNA sequences of dopamine-beta-hydroxylase 19 RNA antisense
cuugaaaagcagggucaggccuu

SEQ ID NO: 290 - RNA, 25 nt, Artificial Sequence
  siRNA sequences of dopamine-beta-hydroxylase 20 RNA antisense
cucuugaaaagcagggucaggccuu

SEQ ID NO: 291 - RNA, 21 nt, Artificial Sequence
  siRNA sequences of dopamine-beta-hydroxylase 21 RNA antisense
uugaaaagcagggucaggccu

SEQ ID NO: 292 - RNA, 21 nt, Artificial Sequence
  siRNA sequences of dopamine-beta-hydroxylase 22 RNA antisense
gcaggugccaaagggccucuu

SEQ ID NO: 293 - RNA, 21 nt, Artificial Sequence
  siRNA sequences of dopamine-beta-hydroxylase 23 RNA antisense
uuggggucgcaggugccaaag

SEQ ID NO: 294 - RNA, 21 nt, Artificial Sequence
  siRNA sequences of dopamine-beta-hydroxylase 24 RNA antisense
uucaaugagguaauccuuggg

SEQ ID NO: 295 - RNA, 21 nt, Artificial Sequence
  siRNA sequences of dopamine-beta-hydroxylase 25 RNA antisense
gucuucaaugagguaauccuu

SEQ ID NO: 296 - RNA, 21 nt, Artificial Sequence
  siRNA sequences of dopamine-beta-hydroxylase 26 RNA antisense
ccaaguggacagugccgucuu

SEQ ID NO: 297 - RNA, 21 nt, Artificial Sequence
  siRNA sequences of dopamine-beta-hydroxylase 27 RNA antisense
uugauggccuccagugaccgg

SEQ ID NO: 298 - RNA, 21 nt, Artificial Sequence
  siRNA sequences of dopamine-beta-hydroxylase 28 RNA antisense
caucugcaggcccgagccguu

SEQ ID NO: 299 - RNA, 21 nt, Artificial Sequence
  siRNA sequences of dopamine-beta-hydroxylase 29 RNA antisense
uucaggagcugcacccucugc

SEQ ID NO: 300 - RNA, 21 nt, Artificial Sequence
  siRNA sequences of dopamine-beta-hydroxylase 30 RNA antisense
uugggcuucaggagcugcacc

SEQ ID NO: 301 - RNA, 21 nt, Artificial Sequence
  siRNA sequences of dopamine-beta-hydroxylase 31 RNA antisense
uucggggauauugggcuucag

SEQ ID NO: 302 - RNA, 21 nt, Artificial Sequence
  siRNA sequences of dopamine-beta-hydroxylase 32 RNA antisense
cgguucggggauauugggcuu

SEQ ID NO: 303 - RNA, 21 nt, Artificial Sequence
  siRNA sequences of dopamine-beta-hydroxylase 33 RNA antisense
caacuccgguucggggauauu

SEQ ID NO: 304 - RNA, 21 nt, Artificial Sequence
  siRNA sequences of dopamine-beta-hydroxylase 34 RNA antisense
cgucugagggcaacuccgguu

SEQ ID NO: 305 - RNA, 21 nt, Artificial Sequence
  siRNA sequences of dopamine-beta-hydroxylase 35 RNA antisense
uuggaccuccauggugcacgc

SEQ ID NO: 306 - RNA, 21 nt, Artificial Sequence
  siRNA sequences of dopamine-beta-hydroxylase 36 RNA antisense
uugggagcuuggaccuccaug

SEQ ID NO: 307 - RNA, 21 nt, Artificial Sequence
  siRNA sequences of dopamine-beta-hydroxylase 37 RNA antisense
ggaucuggauauugggagcuu

SEQ ID NO: 308 - RNA, 21 nt, Artificial Sequence
  siRNA sequences of dopamine-beta-hydroxylase 38 RNA antisense
cuggcuggggaucuggauauu

SEQ ID NO: 309 - RNA, 25 nt, Artificial Sequence
  siRNA sequences of dopamine-beta-hydroxylase 39 RNA antisense
uuaauguagcaccaguacguggucu

SEQ ID NO: 310 - RNA, 23 nt, Artificial Sequence
  siRNA sequences of dopamine-beta-hydroxylase 40 RNA antisense
uuaauguagcaccaguacguggu

SEQ ID NO: 311 - RNA, 21 nt, Artificial Sequence
  siRNA sequences of dopamine-beta-hydroxylase 41 RNA antisense
uuaauguagcaccaguacgug

SEQ ID NO: 312 - RNA, 19 nt, Artificial Sequence
  siRNA sequences of dopamine-beta-hydroxylase 42 RNA antisense
uuaauguagcaccaguacg

SEQ ID NO: 313 - RNA, 21 nt, Artificial Sequence
  siRNA sequences of dopamine-beta-hydroxylase 43 RNA antisense
uuggaagcuccuuaauguagc

SEQ ID NO: 314 - RNA, 21 nt, Artificial Sequence
  siRNA sequences of dopamine-beta-hydroxylase 44 RNA antisense
uuuggaagcuccuuaauguag

SEQ ID NO: 315 - RNA, 21 nt, Artificial Sequence
  siRNA sequences of dopamine-beta-hydroxylase 45 RNA antisense
gaagcccuuuggaagcuccuu

SEQ ID NO: 316 - RNA, 21 nt, Artificial Sequence
  siRNA sequences of dopamine-beta-hydroxylase 46 RNA antisense
uggugccgagagaagcccuuu

SEQ ID NO: 317 - RNA, 21 nt, Artificial Sequence
  siRNA sequences of dopamine-beta-hydroxylase 47 RNA antisense
guggugccgagagaagcccuu

SEQ ID NO: 318 - RNA, 25 nt, Artificial Sequence
  siRNA sequences of dopamine-beta-hydroxylase 48 RNA antisense
uugauaauguggugccgagagaagc

SEQ ID NO: 319 - RNA, 23 nt, Artificial Sequence
  siRNA sequences of dopamine-beta-hydroxylase 49 RNA antisense
uugauaauguggugccgagagaa

SEQ ID NO: 320 - RNA, 21 nt, Artificial Sequence
  siRNA sequences of dopamine-beta-hydroxylase 50 RNA antisense
uugauaauguggugccgagag

SEQ ID NO: 321 - RNA, 19 nt, Artificial Sequence
  siRNA sequences of dopamine-beta-hydroxylase 51 RNA antisense
uugauaauguggugccgag

SEQ ID NO: 322 - RNA, 21 nt, Artificial Sequence
  siRNA sequences of dopamine-beta-hydroxylase 52 RNA antisense
ggugacgaugggcucguacuu

SEQ ID NO: 323 - RNA, 21 nt, Artificial Sequence
  siRNA sequences of dopamine-beta-hydroxylase 53 RNA antisense
uuggugacgaugggcucguac

SEQ ID NO: 324 - RNA, 21 nt, Artificial Sequence
  siRNA sequences of dopamine-beta-hydroxylase 54 RNA antisense
uugcccuuggugacgaugggc

SEQ ID NO: 325 - RNA, 21 nt, Artificial Sequence
  siRNA sequences of dopamine-beta-hydroxylase 55 RNA antisense
gacaagggccucauugcccuu

SEQ ID NO: 326 - RNA, 21 nt, Artificial Sequence
  siRNA sequences of dopamine-beta-hydroxylase 56 RNA antisense
gugguggacaagggccucauu

SEQ ID NO: 327 - RNA, 21 nt, Artificial Sequence
  siRNA sequences of dopamine-beta-hydroxylase 57 RNA antisense
uuccaugugguggacaagggc

SEQ ID NO: 328 - RNA, 21 nt, Artificial Sequence
  siRNA sequences of dopamine-beta-hydroxylase 58 RNA antisense
cgggggcgcacuggaagacuu

SEQ ID NO: 329 - RNA, 21 nt, Artificial Sequence
  siRNA sequences of dopamine-beta-hydroxylase 59 RNA antisense
uuggagucgcagggcccgcug

SEQ ID NO: 330 - RNA, 25 nt, Artificial Sequence
  siRNA sequences of dopamine-beta-hydroxylase 60 RNA antisense
uuucaucuuggagucgcagggcccg

SEQ ID NO: 331 - RNA, 23 nt, Artificial Sequence
  siRNA sequences of dopamine-beta-hydroxylase 61 RNA antisense
uuucaucuuggagucgcagggcc

SEQ ID NO: 332 - RNA, 21 nt, Artificial Sequence
  siRNA sequences of dopamine-beta-hydroxylase 62 RNA antisense
uucaucuuggagucgcagggc

SEQ ID NO: 333 - RNA, 21 nt, Artificial Sequence
  siRNA sequences of dopamine-beta-hydroxylase 63 RNA antisense
uuucaucuuggagucgcaggg

SEQ ID NO: 334 - RNA, 19 nt, Artificial Sequence
  siRNA sequences of dopamine-beta-hydroxylase 64 RNA antisense
uuucaucuuggagucgcag

SEQ ID NO: 335 - RNA, 21 nt, Artificial Sequence
  siRNA sequences of dopamine-beta-hydroxylase 65 RNA antisense
gaggcggucggguuucaucuu

SEQ ID NO: 336 - RNA, 21 nt, Artificial Sequence
  siRNA sequences of dopamine-beta-hydroxylase 66 RNA antisense
uugaggcggucggguuucauc

SEQ ID NO: 337 - RNA, 21 nt, Artificial Sequence
  siRNA sequences of dopamine-beta-hydroxylase 67 RNA antisense
guaguugaggcggucggguuu

SEQ ID NO: 338 - RNA, 21 nt, Artificial Sequence
  siRNA sequences of dopamine-beta-hydroxylase 68 RNA antisense
aguaguugaggcggucggguu

SEQ ID NO: 339 - RNA, 21 nt, Artificial Sequence
  siRNA sequences of dopamine-beta-hydroxylase 69 RNA antisense
cagcacguggcggcaguaguu

SEQ ID NO: 340 - RNA, 21 nt, Artificial Sequence
  siRNA sequences of dopamine-beta-hydroxylase 70 RNA antisense
uuggcacccagggcccaggcg

SEQ ID NO: 341 - RNA, 21 nt, Artificial Sequence
  siRNA sequences of dopamine-beta-hydroxylase 71 RNA antisense
cucuggguaguaaaaugccuu

SEQ ID NO: 342 - RNA, 21 nt, Artificial Sequence
  siRNA sequences of dopamine-beta-hydroxylase 72 RNA antisense
uuccucuggguaguaaaaugc

SEQ ID NO: 343 - RNA, 21 nt, Artificial Sequence
  siRNA sequences of dopamine-beta-hydroxylase 73 RNA antisense
ccccgaaggcaaggccggcuu

SEQ ID NO: 344 - RNA, 21 nt, Artificial Sequence
  siRNA sequences of dopamine-beta-hydroxylase 74 RNA antisense
uuccaggcggagauaucugga

SEQ ID NO: 345 - RNA, 21 nt, Artificial Sequence
  siRNA sequences of dopamine-beta-hydroxylase 75 RNA antisense
uugugguagugaacuuccagg

SEQ ID NO: 346 - RNA, 21 nt, Artificial Sequence
  siRNA sequences of dopamine-beta-hydroxylase 76 RNA antisense
guggguugugguagugaacuu

SEQ ID NO: 347 - RNA, 21 nt, Artificial Sequence
  siRNA sequences of dopamine-beta-hydroxylase 77 RNA antisense
uucuaucaccaguggguugug

SEQ ID NO: 348 - RNA, 21 nt, Artificial Sequence
  siRNA sequences of dopamine-beta-hydroxylase 78 RNA antisense
uccuucuaucaccaguggguu

SEQ ID NO: 349 - RNA, 21 nt, Artificial Sequence
  siRNA sequences of dopamine-beta-hydroxylase 79 RNA antisense
uucguccuucuaucaccagug

SEQ ID NO: 350 - RNA, 21 nt, Artificial Sequence
  siRNA sequences of dopamine-beta-hydroxylase 80 RNA antisense
uuucguccuucuaucaccagu

SEQ ID NO: 351 - RNA, 21 nt, Artificial Sequence
  siRNA sequences of dopamine-beta-hydroxylase 81 RNA antisense
cugaggagucguuucguccuu

SEQ ID NO: 352 - RNA, 21 nt, Artificial Sequence
  siRNA sequences of dopamine-beta-hydroxylase 82 RNA antisense
cggaugccugaggagucguuu

SEQ ID NO: 353 - RNA, 21 nt, Artificial Sequence
  siRNA sequences of dopamine-beta-hydroxylase 83 RNA antisense
gcggaugccugaggagucguu

SEQ ID NO: 354 - RNA, 21 nt, Artificial Sequence
  siRNA sequences of dopamine-beta-hydroxylase 84 RNA antisense
uuggcuguguaguacaagcgg

SEQ ID NO: 355 - RNA, 21 nt, Artificial Sequence
  siRNA sequences of dopamine-beta-hydroxylase 85 RNA antisense
uugaagcgccgcagcuuggcu

SEQ ID NO: 356 - RNA, 21 nt, Artificial Sequence
  siRNA sequences of dopamine-beta-hydroxylase 86 RNA antisense
cgcguugaagcgccgcagcuu

SEQ ID NO: 357 - RNA, 21 nt, Artificial Sequence
  siRNA sequences of dopamine-beta-hydroxylase 87 RNA antisense
cagcuccaugauccccgcguu

SEQ ID NO: 358 - RNA, 21 nt, Artificial Sequence
  siRNA sequences of dopamine-beta-hydroxylase 88 RNA antisense
uuguccgugcaguagccagug

SEQ ID NO: 359 - RNA, 21 nt, Artificial Sequence
  siRNA sequences of dopamine-beta-hydroxylase 89 RNA antisense
cagugccagcugggugcacuu

SEQ ID NO: 360 - RNA, 21 nt, Artificial Sequence
  siRNA sequences of dopamine-beta-hydroxylase 90 RNA antisense
uucucccagucaggugugugu

SEQ ID NO: 361 - RNA, 21 nt, Artificial Sequence
  siRNA sequences of dopamine-beta-hydroxylase 91 RNA antisense
uuucucccagucaggugugug

SEQ ID NO: 362 - RNA, 21 nt, Artificial Sequence
  siRNA sequences of dopamine-beta-hydroxylase 92 RNA antisense
accagcacugugaccaccuuu

SEQ ID NO: 363 - RNA, 21 nt, Artificial Sequence
  siRNA sequences of dopamine-beta-hydroxylase 93 RNA antisense
gaccagcacugugaccaccuu

SEQ ID NO: 364 - RNA, 21 nt, Artificial Sequence
  siRNA sequences of dopamine-beta-hydroxylase 94 RNA antisense
uucacgaucucccacucccgg

SEQ ID NO: 365 - RNA, 21 nt, Artificial Sequence
  siRNA sequences of dopamine-beta-hydroxylase 95 RNA antisense
uuguccugguucacgaucucc

SEQ ID NO: 366 - RNA, 21 nt, Artificial Sequence
  siRNA sequences of dopamine-beta-hydroxylase 96 RNA antisense
gcuguagugauuguccugguu

SEQ ID NO: 367 - RNA, 21 nt, Artificial Sequence
  siRNA sequences of dopamine-beta-hydroxylase 97 RNA antisense
gaagugagggcuguagugauu

SEQ ID NO: 368 - RNA, 25 nt, Artificial Sequence
  siRNA sequences of dopamine-beta-hydroxylase 98 RNA antisense
uucaacaugcggaucuccuggaagu

SEQ ID NO: 369 - RNA, 23 nt, Artificial Sequence
  siRNA sequences of dopamine-beta-hydroxylase 99 RNA antisense
uucaacaugcggaucuccuggaa

SEQ ID NO: 370 - RNA, 21 nt, Artificial Sequence
  siRNA sequences of dopamine-beta-hydroxylase 100 RNA antisense
uucaacaugcggaucuccugg

SEQ ID NO: 371 - RNA, 19 nt, Artificial Sequence
  siRNA sequences of dopamine-beta-hydroxylase 101 RNA antisense
uucaacaugcggaucuccu

SEQ ID NO: 372 - RNA, 21 nt, Artificial Sequence
  siRNA sequences of dopamine-beta-hydroxylase 102 RNA antisense
uucuucaacaugcggaucucc

SEQ ID NO: 373 - RNA, 21 nt, Artificial Sequence
  siRNA sequences of dopamine-beta-hydroxylase 103 RNA antisense
auggaccgacacgaccuucuu

SEQ ID NO: 374 - RNA, 21 nt, Artificial Sequence
  siRNA sequences of dopamine-beta-hydroxylase 104 RNA antisense
cggauggaccgacacgaccuu

SEQ ID NO: 375 - RNA, 21 nt, Artificial Sequence
  siRNA sequences of dopamine-beta-hydroxylase 105 RNA antisense
uuguacgugcaggaggugaug

SEQ ID NO: 376 - RNA, 21 nt, Artificial Sequence
  siRNA sequences of dopamine-beta-hydroxylase 106 RNA antisense
uucuguguuguacgugcagga

SEQ ID NO: 377 - RNA, 21 nt, Artificial Sequence
  siRNA sequences of dopamine-beta-hydroxylase 107 RNA antisense
cagcucccggucuucuguguu

SEQ ID NO: 378 - RNA, 21 nt, Artificial Sequence
  siRNA sequences of dopamine-beta-hydroxylase 108 RNA antisense
cuguggccagcucccggucuu

SEQ ID NO: 379 - RNA, 21 nt, Artificial Sequence
  siRNA sequences of dopamine-beta-hydroxylase 109 RNA antisense
uugacacacaucuccuccagg

SEQ ID NO: 380 - RNA, 21 nt, Artificial Sequence
  siRNA sequences of dopamine-beta-hydroxylase 110 RNA antisense
gggguaguagugcacguaguu

SEQ ID NO: 381 - RNA, 21 nt, Artificial Sequence
  siRNA sequences of dopamine-beta-hydroxylase 111 RNA antisense
uugcagagcuccagcugcguc

SEQ ID NO: 382 - RNA, 21 nt, Artificial Sequence
  siRNA sequences of dopamine-beta-hydroxylase 112 RNA antisense
gccggcguccacagcgcucuu

SEQ ID NO: 383 - RNA, 21 nt, Artificial Sequence
  siRNA sequences of dopamine-beta-hydroxylase 113 RNA antisense
uucugcaggaagccggcgucc

SEQ ID NO: 384 - RNA, 21 nt, Artificial Sequence
  siRNA sequences of dopamine-beta-hydroxylase 114 RNA antisense
uugaugagguggaaguacuuc

SEQ ID NO: 385 - RNA, 21 nt, Artificial Sequence
  siRNA sequences of dopamine-beta-hydroxylase 115 RNA antisense
guugaugagguggaaguacuu

SEQ ID NO: 386 - RNA, 21 nt, Artificial Sequence
  siRNA sequences of dopamine-beta-hydroxylase 116 RNA antisense
uugaaccuguugaugaggugg

SEQ ID NO: 387 - RNA, 21 nt, Artificial Sequence
  siRNA sequences of dopamine-beta-hydroxylase 117 RNA antisense
uuguugaaccuguugaugagg

SEQ ID NO: 388 - RNA, 21 nt, Artificial Sequence
  siRNA sequences of dopamine-beta-hydroxylase 118 RNA antisense
auccucguuguugaaccuguu

SEQ ID NO: 389 - RNA, 21 nt, Artificial Sequence
  siRNA sequences of dopamine-beta-hydroxylase 119 RNA antisense
ggugcagacauccucguuguu

SEQ ID NO: 390 - RNA, 21 nt, Artificial Sequence
  siRNA sequences of dopamine-beta-hydroxylase 120 RNA antisense
gcaggugcagacauccucguu

SEQ ID NO: 391 - RNA, 21 nt, Artificial Sequence
  siRNA sequences of dopamine-beta-hydroxylase 121 RNA antisense
uuccagggaacagaggugaac

SEQ ID NO: 392 - RNA, 21 nt, Artificial Sequence
  siRNA sequences of dopamine-beta-hydroxylase 122 RNA antisense
uugaaggaguuccagggaaca

SEQ ID NO: 393 - RNA, 21 nt, Artificial Sequence
  siRNA sequences of dopamine-beta-hydroxylase 123 RNA antisense
uacgucgcgguugaaggaguu

SEQ ID NO: 394 - RNA, 21 nt, Artificial Sequence
  siRNA sequences of dopamine-beta-hydroxylase 124 RNA antisense
uucaguacgucgcgguugaag

SEQ ID NO: 395 - RNA, 21 nt, Artificial Sequence
  siRNA sequences of dopamine-beta-hydroxylase 125 RNA antisense
ggccuucaguacgucgcgguu

SEQ ID NO: 396 - RNA, 21 nt, Artificial Sequence
  siRNA sequences of dopamine-beta-hydroxylase 126 RNA antisense
cgcgaagcuguacagggccuu

SEQ ID NO: 397 - RNA, 21 nt, Artificial Sequence
  siRNA sequences of dopamine-beta-hydroxylase 127 RNA antisense
uugcagugcauggagaugggc

SEQ ID NO: 398 - RNA, 21 nt, Artificial Sequence
  siRNA sequences of dopamine-beta-hydroxylase 128 RNA antisense
uuguugcagugcauggagaug

SEQ ID NO: 399 - RNA, 21 nt, Artificial Sequence
  siRNA sequences of dopamine-beta-hydroxylase 129 RNA antisense
gcggacggcugaggacuuguu

SEQ ID NO: 400 - RNA, 21 nt, Artificial Sequence
  siRNA sequences of dopamine-beta-hydroxylase 130 RNA antisense
gaagcggacggcugaggacuu

SEQ ID NO: 401 - RNA, 21 nt, Artificial Sequence
  siRNA sequences of dopamine-beta-hydroxylase 131 RNA antisense
uucacccuggaagcggacggc

SEQ ID NO: 402 - RNA, 21 nt, Artificial Sequence
  siRNA sequences of dopamine-beta-hydroxylase 132 RNA antisense
uuccauucacccuggaagcgg

SEQ ID NO: 403 - RNA, 21 nt, Artificial Sequence
  siRNA sequences of dopamine-beta-hydroxylase 133 RNA antisense
gcaggggcugcagguuccauu

SEQ ID NO: 404 - RNA, 21 nt, Artificial Sequence
  siRNA sequences of dopamine-beta-hydroxylase 134 RNA antisense
uugggcaggggcugcagguuc

SEQ ID NO: 405 - RNA, 21 nt, Artificial Sequence
  siRNA sequences of dopamine-beta-hydroxylase 135 RNA antisense
cuugggcaggggcugcagguu

SEQ ID NO: 406 - RNA, 21 nt, Artificial Sequence
  siRNA sequences of dopamine-beta-hydroxylase 136 RNA antisense
uuccaguguggagaugaccuu

SEQ ID NO: 407 - RNA, 21 nt, Artificial Sequence
  siRNA sequences of dopamine-beta-hydroxylase 137 RNA antisense
ggcacuguggggugggcucuu

SEQ ID NO: 408 - RNA, 21 nt, Artificial Sequence
  siRNA sequences of dopamine-beta-hydroxylase 138 RNA antisense
uucggcccuggcugguggggc

SEQ ID NO: 409 - RNA, 21 nt, Artificial Sequence
  siRNA sequences of dopamine-beta-hydroxylase 139 RNA antisense
acgguggggccagcagggcuu

SEQ ID NO: 410 - RNA, 21 nt, Artificial Sequence
  siRNA sequences of dopamine-beta-hydroxylase 140 RNA antisense
uugcccccaccaaugcugaca

SEQ ID NO: 411 - RNA, 21 nt, Artificial Sequence
  siRNA sequences of dopamine-beta-hydroxylase 141 RNA antisense
uuugcccccaccaaugcugac

(19) United States
(12) Patent Application Publication
(10) Pub. No.: US 2023/0416754 A1
(43) Pub. Date: Dec. 28, 2023

(54) SINA MOLECULES, METHODS OF PRODUCTION AND USES THEREOF

(71) Applicant: PHYZAT BIOPHARMACEUTICALS, LDA, Porto (PT)

(72) Inventor: Patrício Manuel VIEIRA ARAUJO SOARES DA SILVA, Porto (PT)

(21) Appl. No.: 18/254,069

(22) PCT Filed: Nov. 23, 2021
(86) PCT No.: PCT/IB2021/060861

§ 371 Date: May 23, 2023

(30) Foreign Application Priority Data
Nov. 23, 2020 (PT) 116899

(51) Int. Cl. C12N 15/113

(52) U.S. Cl. CPC C12N 15/1137; C12Y 114/17001; C12N 2310/14

(57) ABSTRACT

The present disclosure relates to method of producing and using short interfering nucleic acids (siNAs) for preventing or reversing progressive optical neuropathy associated with the elevation of intraocular pressure due to excessive noradrenergic activation. In particular, this disclosure relates to the method of producing and using siNAs for or reversing progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma. The present disclosure is also directed to interfering RNA duplexes and vectors encoding such interfering RNA duplexes.

### TECHNICAL FIELD

The present disclosure relates to a method of producing and using short interfering nucleic acids (siNAs) for preventing and treating ophthalmic diseases. In particular, the method of producing and using siNAs for preventing and treating optical neuropathy associated with the elevation of intraocular pressure due to excessive noradrenergic activation by mediating gene silencing of dopamine-beta-hydroxylase (DBH, EC 1.14.17.1).

The present disclosure also relates to interfering RNA duplexes and vectors encoding such interfering RNA duplexes.

### BACKGROUND

Glaucoma, the main cause of blindness in industrialized countries, is characterized by progressive optic neuropathy and irreversible visual field loss (Prokofyeva & Zrenner, 2012). Risk factors for developing glaucoma include elevated intraocular pressure (IOP), family history, ethnic background, and old age (Coleman & Miglior, 2008; Webers, Beckers, Nuijts & Schouten, 2008). Lowering IOP reduces the progression of nerve damage and therefore therapeutic management of glaucoma includes medications or surgeries that decrease IOP.

Primary open-angle glaucoma is one of the leading causes of irreversible blindness worldwide. It is characterized by a progressive loss of retinal ganglion cells (RGCs) and visual field damage (Quigley, 2011). Many studies have identified high IOP as the most important risk factor for retinal ganglion cell apoptosis in glaucoma (Davis, Crawley, Pahlitzsch, Javaid & Cordeiro, 2016). Clinical evidence has already shown that neuronal damage in glaucoma involves central stations of the visual pathway (Nucci et al., 2013). Recent magnetic resonance imaging studies have also suggested that primary open-angle glaucoma abnormalities are not limited to RGCs but extends to the entire visual pathway as well as some non-visual pathways (Frezzotti et al., 2014; Frezzotti, Giorgio, Toto, De Leucio & De Stefano, 2016).

Different subtypes of adrenoceptors appear to mediate the various ways noradrenaline could affect the progression of open-angle glaucoma. For example, noradrenaline has a constrictive effect on dog ocular arteries, possibly by acting through alpha1-adrenoceptors (Okamura, Fujioka & Ayajiki, 2002). The alpha2 adrenoceptor agonists, xylazine and clonidine, both produce mydriasis (excessive pupil dilation, which can reduce the drainage angle), possibly through postsynaptic agonism of alpha2 adrenoceptors (Hey, Gherezghiher & Koss, 1985; Hsu, Lee & Betts, 1981). Endogenous noradrenaline may act in a similar fashion through these receptors. Pharmacological blockade of beta adrenoceptors decreases aqueous humour production (Rittenhouse & Pollack, 2000), suggesting that if endogenous noradrenaline boosts humour production, it may do so through stimulation of beta adrenoceptors. Timolol significantly reduced IOP in saline control treated mice, but did not significantly affect IOP in the reserpine-treated animals. These findings demonstrate that catecholamines are required for the effects of the beta adrenoceptor antagonist, timolol (Ishikawa, Yoshitomi, Zorumski & Izumi, 2015).

A second line of evidence for an etiological role of noradrenaline in open-angle glaucoma comes from studies of noradrenergic drugs in humans. The main point here is that two major classes of drugs that are used to treat open-angle glaucoma, beta-blockers and alpha2 adrenergic agonists, exert their effects directly on noradrenaline signaling. Examples of these drugs include the beta-blocker timolol and the alpha2-adrenoceptor agonist brimonidine, both mentioned above in preclinical studies (Burke et al., 1995; Greenfield, Liebmann & Ritch, 1997; Gupta, Agarwal, Galpalli, Srivastava, Agrawal & Saxena, 2007; Seki et al., 2005). Brimonidine has been widely used to treat open-angle glaucoma, and it is also used in combination with timolol (Fudemberg, Batiste & Katz, 2008).

Noradrenaline may be involved in other types of glaucoma as well. A first line of evidence involves rodent studies of glaucoma in the context of pharmacological noradrenaline manipulation. Drugs that affect noradrenaline signaling have been demonstrated to affect IOP in rodents as well as in rabbits. A number of studies have tested beta-blockers, such as timolol, in these animal models and found a reduction in IOP (Gupta, Agarwal, Galpalli, Srivastava, Agrawal & Saxena, 2007; Seki et al., 2005). Other studies have tested noradrenaline lowering alpha2-adrenoceptor agonists, such as brimonidine, and found a reduction in IOP (Burke et al., 1995; Greenfield, Liebmann & Ritch, 1997). In addition, brimonidine and clonidine exert neuroprotective effects on the retina (Ahmed, Hegazy, Chaudhary & Sharma, 2001; Wheeler & Woldemussie, 2001).

Given that excessive production of noradrenaline may be involved in open angle glaucoma and other types of glaucoma, it is hypothesized that inhibition of excessive noradrenaline production might result in a valid alternative to therapies based on the modulation of noradrenaline-mediated effects through blockade of beta-adrenoceptors or activation of alpha2-adrenoceptors which lead to decreases in noradrenaline release from sympathetic nerve endings, as these do not alter the root cause of eye sympathetic noradrenergic overactivity.

The eye is a relatively isolated tissue compartment; this provides several advantages to the use of siRNA-based therapies. Local delivery of compounds to the eye limits systemic exposure and reduces the amount of compound needed. It allows for local silencing of a gene while reducing the likelihood of wide spread silencing outside the eye. In addition, the immune system has limited access to the eye; therefore, immune responses to the compound are less likely to occur (Campochiaro, 2006). Finally, the eye has lower content in RNases than other tissues, allowing for an increased stability of RNA-based compounds (Martinez, Gonzalez, Roehl, Wright, Paneda & Jimenez, 2014).

RNA interference (“RNAi”) is a recently discovered mechanism of post-transcriptional gene silencing in which double-stranded RNA corresponding to a gene (or coding region) of interest is introduced into an organism, resulting in degradation of the corresponding mRNA. The phenomenon was originally discovered in Caenorhabditis elegans (Fire, Xu, Montgomery, Kostas, Driver & Mello, 1998).

Unlike antisense technology, the RNAi phenomenon persists for multiple cell divisions before gene expression is regained. The process occurs in at least two steps: an endogenous ribonuclease cleaves the longer dsRNA into shorter, 21-22- or 23-nucleotide-long RNAs, termed “small interfering RNAs” or siRNAs (Hannon, 2002). The siRNA segments then mediate the degradation of the target mRNA. RNAi has been used for gene function determination in a manner similar to but more efficient than antisense oligonucleotides. By making targeted knockouts at the RNA level by RNAi, rather than at the DNA level using conventional gene knockout technology, a vast number of genes can be assayed quickly and efficiently. RNAi is therefore an extremely powerful, simple method for assaying gene function.

RNAi has been shown to be effective in cultured mammalian cells. In most methods described to date, RNAi is carried out by introducing double-stranded RNA into cells by microinjection or by soaking cultured cells in a solution of double-stranded RNA, as well as transfecting the cells with a plasmid carrying a hairpin-structured siRNA expressing cassette under the control of suitable promoters, such as the U6, H1 or cytomegalovirus (“CMV”) promoter (Brummelkamp, Bernards & Agami, 2002; Elbashir, Harborth, Lendeckel, Yalcin, Weber & Tuschl, 2001; Harborth, Elbashir, Bechert, Tuschl & Weber, 2001; Lee et al., 2001; Miyagishi & Taira, 2002; Paddison, Caudy, Bernstein, Hannon & Conklin, 2002; Paul, Good, Winer & Engelke, 2002; Sui et al., 2002; Xia, Mao, Paulson & Davidson, 2002; Yu, DeRuiter & Turner, 2002). The gene-specific inhibition of gene expression by double-stranded ribonucleic acid is generally described in U.S. Pat. No. 6,506,559, which is incorporated herein by reference. Exemplary use of siRNA technology is further described in Published U.S. Patent Application No. 2003/01090635 and Published U.S. Patent Application No. 20040248174, which are incorporated herein by reference. Davis (Davis, 2009) describes the targeted delivery of siRNA to humans using nanoparticle technology.

These facts are disclosed in order to illustrate the technical problem addressed by the present disclosure.

### GENERAL DESCRIPTION

The present disclosure relates to method of producing and using short interfering nucleic acids (siNAs) for preventing and treating ophthalmic diseases. In particular, the method of producing and using siRNAs for preventing and treating optical neuropathy associated with the elevation of intraocular pressure due to excessive noradrenergic activation by mediating gene silencing of dopamine-beta-hydroxylase (DBH, EC 1.14.17.1), the last enzymatic step in the synthesis of the noradrenergic neurotransmitter noradrenaline. The present disclosure also relates to interfering RNA duplexes and vectors encoding such interfering RNA duplexes.

In an embodiment, the present disclose further relates to use of RNA interference (RNAi) to downregulate the expression of dopamine-beta-hydroxylase (EC 1.14.17.1), as an advantageous therapeutic approach to glaucoma. The RNAi of the present disclosure specifically and selectively addresses the root cause of excessive eye sympathetic noradrenergic overactivity and serves as a local ocular therapy with a sustained effect over time.

In an embodiment, the present disclosure relates to the use of specific short interfering nucleic acid molecules (siNA) to downregulate the expression of the gene for dopamine-beta-hydroxylase in order to treat or prevent ophthalmic diseases associated with the elevation of intraocular pressure (IPO) due to excessive noradrenergic activation.

In an embodiment, the compositions (or molecules) of the present disclosure comprises short interfering nucleic acid molecules

(siNA) and related nucleic acids such as short interfering RNA (siRNA), double-stranded RNA (dsRNA), micro-RNA (miRNA), antagomirs and short hairpin RNA (shRNA) capable of mediating RNA interference.

In an embodiment, the siNA of the disclosure is incorporated into RNA-induced silencing complex (RISC).

In an embodiment, the siRNA of the present disclosure down-regulates the expression of dopamine-beta-hydroxylase gene.

In an embodiment, the siNA of the present disclosure specifically targets at least one sequence selected from SEQ ID No 1 to SEQ ID No 137, or a variant thereof.

In an embodiment, the siNA of the present disclosure specifically targets at least one sequence complementary to at least one sequence selected from SEQ ID No 138 to SEQ ID No 274, or a variants thereof.

In an embodiment, the disclosure relates to an isolated siNA molecule, preferably an isolated siRNA molecule.

In one embodiment, the siNA molecule specifically targets at least one sequence selected from SEQ ID No 1, SEQ ID No 4, SEQ ID No 5, SEQ ID No 6, SEQ ID No 7, SEQ ID No 8, SEQ ID No 10, SEQ ID No 13, SEQ ID No 14, SEQ ID No 15, SEQ ID No 16, SEQ ID No 17, SEQ ID No 20, SEQ ID No 21, SEQ ID No 22, SEQ ID No 24, SEQ ID No 33, SEQ ID No 35, SEQ ID No 36, SEQ ID No 37, SEQ ID No 38, SEQ ID No 39, SEQ ID No 40, SEQ ID No 41, SEQ ID No 44, SEQ ID No 45, SEQ ID No 46, SEQ ID No 47, SEQ ID No 48, SEQ ID No 53, SEQ ID No 56, SEQ ID No 57, SEQ ID No 58, SEQ ID No 59, SEQ ID No 60, SEQ ID No 63, SEQ ID No 64, SEQ ID No 67, SEQ ID No 71, SEQ ID No 72, SEQ ID No 73, SEQ ID No 74, SEQ ID No 75, SEQ ID No 76, SEQ ID No 77, SEQ ID No 82, SEQ ID No 83, SEQ ID No 87, SEQ ID No 90, SEQ ID No 92, SEQ ID No 93, SEQ ID No 94, SEQ ID No 95, SEQ ID No 96, SEQ ID No 97, SEQ ID No 98, SEQ ID No 101, SEQ ID No 102, SEQ ID No 105, SEQ ID No 106, SEQ ID No 110, SEQ ID No 111, SEQ ID No 112, SEQ ID No 113, SEQ ID No 114, SEQ ID No 118, SEQ ID No 120, SEQ ID No 124, SEQ ID No 128 and SEQ ID No 132 or a variant thereof. Preferably, the siNA molecule targets a sequence selected from SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132, or a variants thereof.

In an embodiment, the siNA reduces the expression of dopamine-beta-hydroxylase protein by dopamine-beta-hydroxylase gene expression in a cell.

In an embodiment, the siNA preferably comprises a double-stranded RNA molecule, wherein the antisense strand is substantially complementary to any of SEQ ID No 1, SEQ ID No 4, SEQ ID No 5, SEQ ID No 6, SEQ ID No 7, SEQ ID No 8, SEQ ID No 10, SEQ ID No 13, SEQ ID No 14, SEQ ID No 15, SEQ ID No 16, SEQ ID No 17, SEQ ID No 20, SEQ ID No 21, SEQ ID No 22, SEQ ID No 24, SEQ ID No 33, SEQ ID No 35, SEQ ID No 36, SEQ ID No 37, SEQ ID No 38, SEQ ID No 39, SEQ ID No 40, SEQ ID No 41, SEQ ID No 44, SEQ ID No 45, SEQ ID No 46, SEQ ID No 47, SEQ ID No 48, SEQ ID No 53, SEQ ID No 56, SEQ ID No 57, SEQ ID No 58, SEQ ID No 59, SEQ ID No 60, SEQ ID No 63, SEQ ID No 64, SEQ ID No 67, SEQ ID No 71, SEQ ID No 72, SEQ ID No 73, SEQ ID No 74, SEQ ID No 75, SEQ ID No 76, SEQ ID No 77, SEQ ID No 82, SEQ ID No 83, SEQ ID No 87, SEQ ID No 90, SEQ ID No 92, SEQ ID No 93, SEQ ID No 94, SEQ ID No 95, SEQ ID No 96, SEQ ID No 97, SEQ ID No 98, SEQ ID No 101, SEQ ID No 102, SEQ ID No 105, SEQ ID No 106, SEQ ID No 110, SEQ ID No 111, SEQ ID No 112, SEQ ID No 113, SEQ ID No 114, SEQ ID No 118, SEQ ID No 120, SEQ ID No 124, SEQ ID No 128 and SEQ ID No 132 or a variant thereof, even more preferably SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132, and its sense strand comprises an RNA sequence complementary to the sense strand, wherein both strands are hybridized by standard base pairing between nucleotides.

In an embodiment, said sense stand comprises a sequence selected from SEQ ID No 138, SEQ ID No 141, SEQ ID No 142, SEQ ID No 143, SEQ ID No 144, SEQ ID No 145, SEQ ID No 147, SEQ ID No 150, SEQ ID No 151, SEQ ID No 152, SEQ ID No 153, SEQ ID No 154, SEQ ID No 157, SEQ ID No 158, SEQ ID No 159, SEQ ID No 161, SEQ ID No 170, SEQ ID No 172, SEQ ID No 173, SEQ ID No 174, SEQ ID No 175, SEQ ID No 176, SEQ ID No 177, SEQ ID No 178, SEQ ID No 179, SEQ ID No 180, SEQ ID No 181, SEQ ID No 182, SEQ ID No 183, SEQ ID No 184, SEQ ID No 185, SEQ ID No 190, SEQ ID No 193, SEQ ID No 194, SEQ ID No 195, SEQ ID No 196, SEQ ID No 197, SEQ ID No 200, SEQ ID No 201, SEQ ID No 204, SEQ ID No 208, SEQ ID No 209, SEQ ID No 210, SEQ ID No 211, SEQ ID No 212, SEQ ID No 213, SEQ ID No 214, SEQ ID No 219, SEQ ID No 220, SEQ ID No 224, SEQ ID No 227, SEQ ID No 229, SEQ ID No 230, SEQ ID No 231, SEQ ID No 232, SEQ ID No 233, SEQ ID No 234, SEQ ID No 235, SEQ ID No 238, SEQ ID No 239, SEQ ID No 242, SEQ ID No 243, SEQ ID No 247, SEQ ID No 248, SEQ ID No 249, SEQ ID No 250, SEQ ID No 251, SEQ ID No 255, SEQ ID No 257, SEQ ID No 261, SEQ ID No 265 and SEQ ID No 269, more preferably SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265 and SEQ ID No 269, or a variants thereof.

In an embodiment, said antisense strand comprises a sequence selected from SEQ ID No 275, SEQ ID No 278, SEQ ID No 279, SEQ ID No 280, SEQ ID No 281, SEQ ID No 282, SEQ ID No 284, SEQ ID No 287, SEQ ID No 288, SEQ ID No 289, SEQ ID No 290, SEQ ID No 291, SEQ ID No 294, SEQ ID No 295, SEQ ID No 296, SEQ ID No 298, SEQ ID No 307, SEQ ID No 309, SEQ ID No 310, SEQ ID No 311, SEQ ID No 312, SEQ ID No 313, SEQ ID No 314, SEQ ID No 315, SEQ ID No 316, SEQ ID No 317, SEQ ID No 318, SEQ ID No 319, SEQ ID No 320, SEQ ID No 321, SEQ ID No 322, SEQ ID No 327, SEQ ID No 330, SEQ ID No 331, SEQ ID No 332, SEQ ID No 333, SEQ ID No 334, SEQ ID No 337, SEQ ID No 338, SEQ ID No 341, SEQ ID No 345, SEQ ID No 346, SEQ ID No 347, SEQ ID No 348, SEQ ID No 349, SEQ ID No 350, SEQ ID No 351, SEQ ID No 356, SEQ ID No 357, SEQ ID No 361, SEQ ID No 364, SEQ ID No 366, SEQ ID No 367, SEQ ID No 368, SEQ ID No 369, SEQ ID No 370, SEQ ID No 371, SEQ ID No 372, SEQ ID No 375, SEQ ID No 376, SEQ ID No 379, SEQ ID No 380, SEQ ID No 384, SEQ ID No 385, SEQ ID No 386, SEQ ID No 387, SEQ ID No 388, SEQ ID No 392, SEQ ID No 394, SEQ ID No 398, SEQ ID No 402 and SEQ ID No 406, more preferably SEQ ID No 279, SEQ ID No 280, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406, or a variants thereof.

In an embodiment, the variant of at least one sequence is selected from SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265 and SEQ ID No 269, has at least a 95% overall sequence identity, preferably at least 96%, 97%, 98%, 99% identical.

In an embodiment, the variant of at least one sequence is selected from SEQ ID No 279, SEQ ID No 280, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406, has at least a 95% overall sequence identity, preferably at least 96%, 97%, 98%, 99% identical.

Within the meaning of the present disclosure “substantially complementary” to a target mRNA sequence, may also be understood as “substantially identical” to said target sequence. “Identity” as is known by one of ordinary skill in the art, is the degree of sequence relatedness between nucleotide sequences as determined by matching the order and identity of nucleotides between sequences.

In an embodiment, the antisense strand of the siRNA of the present disclosure is 80% complementary to the target mRNA sequence and is considered substantially complementary.

In an embodiment, the antisense strand of the siRNA of the present disclosure is from 80% to 100% complementary to the target mRNA sequence and is considered substantially complementary.

In an embodiment, the antisense strand of the siRNA of the present disclosure is 85%, 86%, 87%, 88%, 89%, 90%, 91%, 92%, 93%, 94%, 95%, 96%, 97% or 99% complementary to the target mRNA sequence and is considered substantially complementary.

The percentage of complementarity describes the percentage of contiguous nucleotides in a first nucleic acid molecule that can base pair in the Watson-Crick sense with a set of contiguous nucleotides in a second nucleic acid molecule.

A gene is “targeted” by a siNA according to the present disclosure when, for example, the siNA molecule selectively decreases or inhibits the expression of the gene. The phrase “selectively decrease or inhibit” as used herein encompasses siNAs that affect expression of the dopamine-beta-hydroxylase protein. Alternatively, a siNA targets a gene when the siNA hybridizes under stringent conditions to the gene transcript, i.e. its mRNA. Capable of hybridizing “under stringent conditions” means annealing to the target mRNA region, under standard conditions, e.g., high temperature and/or low salt content which tend to disfavor hybridization. A suitable protocol (involving 0.1×SSC, 68° C. for 2 hours) is described in Maniatis, T., et al., Molecular Cloning: A Laboratory Manual, Cold Spring Harbor Laboratory, 1982, at pages 387-389.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

In an embodiment, the siNA of the present disclosure is 40 base pairs in length,

In an embodiment, the siNA of the present disclosure is less than 40 base pairs in length, preferably, 19 to 25 base pairs in length.

In an embodiment, the siNA comprises a 21-nucleotide double-stranded region, preferably, the siNA has a sense and an anti-sense strand.

In an embodiment, the siNA molecule comprises a 19-nucleotide double-stranded region.

In an embodiment, the siNA has blunt ends.

In an embodiment, the siNA has 5′ and/or 3′ overhangs, preferably the overhangs are between 1 to 5 nucleotides, more preferably, 2 nucleotides overhangs.

In an embodiment, the overhangs are ribonucleic acids or deoxyribonucleic acids.

In an embodiment, the siNA molecule of the present disclosure comprises a chemical modification, preferably, the chemical modification is on the sense strand, the antisense strand or on both strands.

Phosphorothioate (PS)- or boranophosphate (BS)-modified siRNAs have substantial nuclease resistance. Silencing by siRNA duplexes is also compatible with some types of 2′-sugar modifications: 2′-H,2′-O-methyl, 2′-O-methoxyethyl, 2′-fluoro (2′-F), locked nucleic acid (LNA) and ethylene-bridge nucleic acid (ENA).

In an embodiment, the 5′ or 3′ overhangs are dinucleotides, preferably thymidine dinucleotide.

In an embodiment, the 5′ or 3′ overhangs are deoxythymidines.

In an embodiment, the sense strand comprises at least one 3′ overhangs, preferably two 3′ overhangs.

In an embodiment, said sense strand comprises at least one 3′ deoxythymidines, preferably two 3′ deoxythymidines.

In an embodiment, the antisense strand comprises at least one 3′ overhangs, preferably two 3′ overhangs.

In an embodiment, said sense strand comprises at least one 3′ deoxythymidines, preferably two 3′ deoxythymidines.

In an embodiment, both the sense and antisense strands comprise 3′ overhangs.

“Variant” as used herein meant a sequence with 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic or ribonucleic acid sequence.

“Down-regulating” as used herein meant a decrease in the expression of dopamine-beta-hydroxylase protein from DBH mRNA by up to or more than 10%, 15% 20%, 25%, 30%, 35%, 40%, 45% 50%, 55% 60%, 65%, 70%, 75%, 80%, 85%, 90%, 95% as compared to the level in a control. Alternatively, the siNA molecule described herein may abolish dopamine-beta-hydroxylase protein expression. The term “abolish” means that no expression of dopamine-beta-hydroxylase protein is detectable or that no functional dopamine-beta-hydroxylase protein is produced. For example, a reduction in the expression and/or protein levels of at least dopamine-beta-hydroxylase protein expression may be a measure of protein and/or nucleic acid levels and can be measured by any technique known to the skilled person, such as, but not limited to, any form of gel electrophoresis or chromatography (e.g. HPLC).

In an embodiment, the siNA (either the 5′ or 3′ strand or both) may begin with at least one alanine nucleotide, preferably two alanine nucleotides.

In an embodiment, if the target sequence starts with one or two alanine sequences, these may not be included (targeted) in the siNA of the present disclosure.

In an embodiment, the target sequence may be characterized by at least one alanine nucleotides at the 3′ end, preferably two alanine nucleotides at the 3′ end of the sequence.

In an embodiment, the target sequence lacks at least one alanine nucleotides at the 5′ end, preferably two alanine nucleotides at the 5′ end of the sequence.

In an embodiment, the target sequence lacks two consecutive alanine nucleotides within the sequence.

In a preferred embodiment, the siNA molecules of the present disclosure are characterized in that they target sequences which lacks at least one alanine nucleotides at the 3′ end, or preferably lacks two alanine nucleotides at the 3′ end, or lacks at least one alanine nucleotides at the 5′ end, or preferably lacks two alanine nucleotides at the 5′ end, or lacks two consecutive alanine nucleotides within the sequence.

In an embodiment a plurality of species of siNA molecule are used, wherein said plurality of siNA molecules are targeted to the same or different mRNA species.

In an embodiment, the siNA is selected from dsRNA, siRNA or shRNA. Preferably, the siNA is a siRNA.

In an embodiment, the isolated or synthetic siNA comprises a sequence at least 88% identical to SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132.

In an embodiment, the isolated or synthetic siNA comprises preferably a sequence at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132.

In an embodiment, the isolated or synthetic siNA comprises a sequence at least 88% identical SEQ ID No 279, SEQ ID No 280, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

In an embodiment, the isolated or synthetic siNA comprises a sequence preferably at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 279, SEQ ID No 280, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

An aspect of the present disclosure relates to an isolated or synthetic short interfering nucleic acid—siNA—molecule, wherein said molecule comprises a nucleic acid sequence selected from a list consisting of SEQ ID No 144, SEQ ID No 174, SEQ ID No 233, SEQ ID No 235, SEQ ID No 239, SEQ ID No 250, SEQ ID No 265 and SEQ ID No 269 and/or a nucleic acid sequence selected from a list consisting of SEQ ID No 281, SEQ ID No 311, SEQ ID No 370, SEQ ID No 372, SEQ ID No 376, SEQ ID No 387, SEQ ID No 402 or SEQ ID No 406 and/or sequences comprising at least 18 contiguous nucleotides differing by no more than 4 nucleotides from the nucleotide sequence, and wherein said siNA molecule reduces expression of the dopamine-beta-hydroxylase—DBH—gene in a cell.

In an embodiment, the siNA comprises a nucleic acid sequence differing by no more than 3 nucleotides from the nucleotide sequence; preferably the siNA molecule may comprises a nucleic acid sequence differing by no more than 2 nucleotides from the nucleotide sequence; more preferably a nucleic acid sequence differing by no more than 1 nucleotides from the.

In an embodiment, siNA is between 19 and 25 base pairs in length; preferably between 21 and 23 base pairs in length.

In an embodiment, siNA may comprises a nucleic acid sequence selected from a list consisting of SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265 and SEQ ID No 269; and/or SEQ ID No 279, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

Another aspect of the present disclosure relates to a double stranded ribonucleic acid (dsRNA) agent for inhibiting expression of DBH-gene in a cell, wherein the dsRNA agent comprises a sense strand and an antisense strand forming a double stranded region, wherein the sense strand comprises a nucleic acid sequence selected from a list consisting of SEQ ID No 144, SEQ ID No 174, SEQ ID No 233, SEQ ID No 235, SEQ ID No 239, SEQ ID No 250, SEQ ID No 265 and SEQ ID No 269; or sequences comprising at least 18 contiguous nucleotides differing by no more than 4 nucleotides from the nucleotide sequence, and wherein the antisense strand comprises a nucleic acid sequence selected from a list consisting of SEQ ID No 281, SEQ ID No 311, SEQ ID No 370, SEQ ID No 372, SEQ ID No 376, SEQ ID No 387, SEQ ID No 402 and SEQ ID No 406; or sequences comprising at least 18 contiguous nucleotides differing by no more than 4 nucleotides from the nucleotide sequence.

In an embodiment, the dsRNA agent may comprise a nucleic acid sequence differing by no more than 3 nucleotides from the nucleotide sequence; no more than 2 nucleotides from the nucleotide sequence; more preferably no more than 1 nucleotides from the nucleotide sequence.

In an embodiment, the sense strand is selected from a list consisting of SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265 and SEQ ID No 269 and the anti-sense strand is selected from a list consisting of SEQ ID No 279, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

In an embodiment, the siNA of the present disclosure is for use as a medicament.

In an embodiment, the siNA of the present disclosure is for use in the treatment of disorders associated with increased expression levels (compared to the levels in a healthy subject) of dopamine-beta-hydroxylase protein.

In an embodiment, the siNA of the present disclosure is for use in preventing or reversing progressive optical neuropathy associated with elevation of intraocular pressure due to excessive noradrenergic activation.

In an embodiment, the siNA of the present disclosure is for use in the preparation of or as a medicament for preventing or reversing progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the siRNA of the present disclosure is for use as a medicament for preventing or reversing progressive optical neuropathy associated with the elevation of intraocular pressure due to excessive noradrenergic activation.

In an embodiment, the method for preventing or reversing progressive optical neuropathy associated with the elevation of intraocular pressure due to excessive noradrenergic activation comprises administering at least one siNA molecule, as described herein, to a patient or subject in need thereof.

In an embodiment, the optical neuropathy disorder is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the disclosure relates to a pharmaceutical composition comprising at least one siNA of the present disclosure and a pharmaceutically acceptable carrier.

In an embodiment, the siNA of the present disclosure inhibits the in vitro expression of dopamine-beta-hydroxylase protein expression. In vitro dopamine-beta-hydroxylase protein expression is inhibited by administering a siNA of the present disclosure into a cell.

In an embodiment, the in vitro dopamine-beta-hydroxylase expression in a cell is inhibited by up to or more than 10%, 20%, 30%, 40%, 50%, 60%, 70%, 80% or 90% as compared to the level in a control.

In an embodiment, the siNA of the present disclosure inhibits the in vitro activity of dopamine-beta-hydroxylase. In vitro dopamine-beta-hydroxylase activity is inhibited by administering a siNA of the present disclosure into a cell.

In an embodiment, the dopamine-beta-hydroxylase activity is inhibited by up to or more than 10%, 20%, 30%, 40%, 50%, 60%, 70%, 80% or 90% as compared to the level in a control.

In an embodiment, the present disclosure relates to a method of reducing dopamine-beta-hydroxylase protein expression and activity, preferably in a patient, the method comprising administering at least one siNA of the present disclosure.

In an embodiment, the decrease in dopamine-beta-hydroxylase protein expression and activity may be up to or more than 10%, 20%, 30%, 40%, 50%, 60%, 70%, 80% or 90% as compared to the level in a control.

In an embodiment, the disclosure relates to methods of reducing dopamine-beta-hydroxylase protein expression and activity in a cell comprising treating the cells with an siNA of the disclosure in combination with one or more agents known in the art, preferably wherein the agent comprises an anti-glaucoma agent and most preferably alpha adrenoceptor agonists (apraclonidine, brimonidine), beta adrenoceptor blockers (betaxolol, levobunolol, metpranolol, timolol), carbonic anhydrase inhibitors (acetazolamide, brinzolamide, dorzolamide, methazolamide), muscarinic agonists (carbachol, pilocarpine), prostaglandin analogs (bimatoprost, latanoprost, tafluprost, travaprost), rho kinase inhibitors (netarsudil).

In an embodiment, the present disclosure also relates to methods of preventing or reversing progressive optical neuropathy associated with the elevation of intraocular pressure due to excessive noradrenergic activation comprising administrating an siNA of the present disclosure in combination with one or more anti-glaucoma agents known in the art, preferably to a patient in need thereof.

In an embodiment, the anti-glaucoma agent comprises an anti-glaucoma agent, more preferably an alpha adrenoceptor agonists, beta adrenoceptor blockers, carbonic anhydrase inhibitors, muscarinic agonists, prostaglandin analogs and rho kinase inhibitors agent and most preferably apraclonidine, brimonidine, betaxolol, levobunolol, metpranolol, timolol, acetazolamide, brinzolamide, dorzolamide, methazolamide, carbachol, pilocarpine, bimatoprost, latanoprost, tafluprost, travaprost, and netarsudil.

In an embodiment, the disclosure further relates to pharmaceutical compositions comprising the siNA of the present disclosure and one or more anti-glaucoma agent.

In an embodiment, the disclosure relates to methods for increasing the efficacy of an anti-glaucoma therapy given to a patient. The method comprising administering an siNA of the present disclosure in combination with the therapy.

In an embodiment, the increase in anti-glaucoma therapy efficacy may be up to or more than 10%, 20%, 30%, 40%, 50%, 60%, 70%, 80% or 90% as compared to the efficacy of either administration of siNA or the anti-glaucoma agent alone.

In an embodiment, the disclosure also relates to methods of treating glaucoma comprising administrating an siNA of the present disclosure in combination with one or more types of laser surgery known in the art to treat glaucoma, preferably to a patient in need thereof.

In an embodiment, the laser surgery comprises trabeculoplasty or iridotomy, trabeculotomy and implantation of glaucoma drainage devices.

In an embodiment, the disclosure further relates to pharmaceutical compositions comprising the siNA of the present disclosure and one or more types of laser surgery, trabeculotomy and implantation of glaucoma drainage devices.

In an embodiment, the disclosure relates to methods for increasing the efficacy of laser surgery, trabeculotomy and implantation of glaucoma drainage devices, performed in a patient comprising administering an siNA of the disclosure in combination with the therapy.

In an embodiment, the increase in laser surgery efficacy may be up to or more than 10%, 20%, 30%, 40%, 50%, 60%, 70%, 80% or 90% when compared to the efficacy of either administration of siNA or the laser surgery (trabeculoplasty or iridotomy), trabeculotomy and implantation of glaucoma drainage devices inhibition therapy alone.

### BRIEF OF THE DRAWINGS

The following figures provide preferred embodiments for illustrating the disclosure and should not be seen as limiting the scope of invention.

FIG. 1. (A) relative abundance of dopamine-beta-hydroxylase mRNA determined by qRT-PCR in SK-N-SH cells 72 h after treatment (6 h) with 25 nM anti-dopamine-beta-hydroxylase siRNAs against nucleotide sequences No 144, 147, 158, 159, 174, 176, 177, 183, 202, 208, 212, 213, 214, 219, 229, 233, 235, 237, 238, 239, 242, 249, 251, 250, 254, 252, 260, 264 and 271 (e.g., an siRNA comprising or consisting of SEQ ID No 281, 284, 295, 296, 311, 313, 314, 320, 339, 345, 349, 350, 351, 356, 366, 370, 372, 374, 375, 376, 379, 386, 388, 387, 391, 389, 397, 401 and 408, respectively) and commercially available anti-dopamine-beta-hydroxylase siRNAs (25 nM) SI00015671 or SI00015624. FIG. 1. (B). Beta hydroxylation of dopamine in SK-N-SH cells 72 h after treatment (for 6 h) with 25 nM anti-dopamine-beta-hydroxylase siRNAs against nucleotide sequences No 144, 147, 158, 159, 174, 176, 177, 183, 202, 208, 212, 213, 214, 219, 229, 233, 235, 237, 238, 239, 242, 249, 251, 250, 254, 252, 260, 264 and 271 (e.g., an siRNA comprising or consisting of SEQ ID No 281, 284, 295, 296, 311, 313, 314, 320, 339, 345, 349, 350, 351, 356, 366, 370, 372, 374, 375, 376, 379, 386, 388, 387, 391, 389, 397, 401 and 408, respectively) and commercially available anti-dopamine-beta-hydroxylase siRNAs (25 nM) SI00015671 or SI00015624 and treatment (for 2 h) with 1 μM nepicastat.

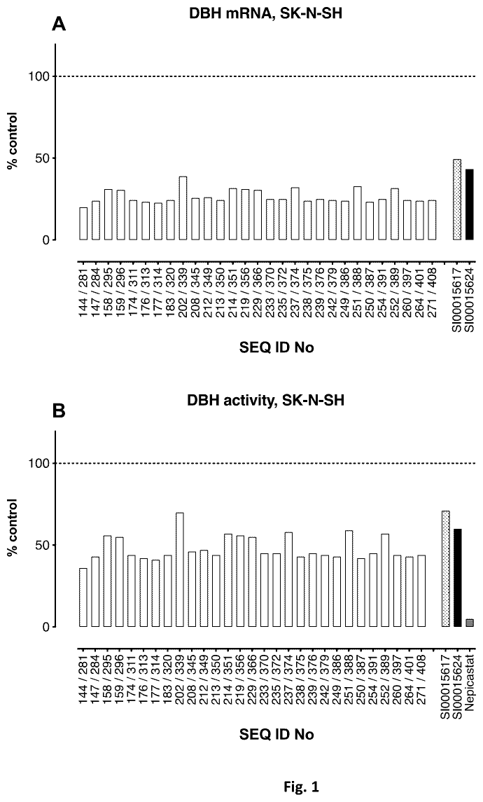

FIG. 2. Beta hydroxylation of dopamine in SK-N-SH cells every 168 hours after treatment (for 6 h) with 25 nM anti-dopamine-beta-hydroxylase siRNA against nucleotide sequences No 233 (e.g., an siRNA comprising or consisting of SEQ ID No 370) and treatment (for 2 h) with 1 μM nepicastat.

FIG. 3. Integrity of a natural (SEQ ID No 144/281, 174/311 and 233/370) or chemically modified (SEQ ID No 144/281F, 174/311F and 233/370F) 21 nucleotide siRNA anti-dopamine-beta-hydroxylase when exposed for 30 in cell culture medium in the absence and the presence of (A) 10% FBS or (B) RNAse I (0.50 Units).

FIG. 4. Relative abundance of dopamine-beta-hydroxylase mRNA in SK-N-SH cells by RT-qPCR (relative to GADPH) after exposure (6 h) to transfection agent (0.3% iMax) and increasing concentrations of a natural (SEQ No ID 233/370) or chemically modified (SEQ ID No 233/370F) 21 nucleotide siRNA anti-dopamine-beta-hydroxylase at 24 h after treatment.

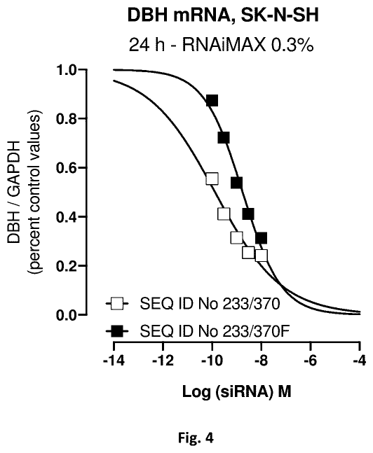

FIG. 5. Relative abundance of dopamine-beta-hydroxylase protein in SK-N-SH cells by western blot (relative to GADPH) after exposure (6 h) to transfection agent (0.4% iMax) 10 nM of a natural (SEQ No ID 233/370) or chemically modified (SEQ ID No 233/370F) 21 nucleotide siRNA anti-dopamine-beta-hydroxylase and commercially available anti-dopamine-beta-hydroxylase siRNA (10 nM) SI00015624 at 72 h after treatment.

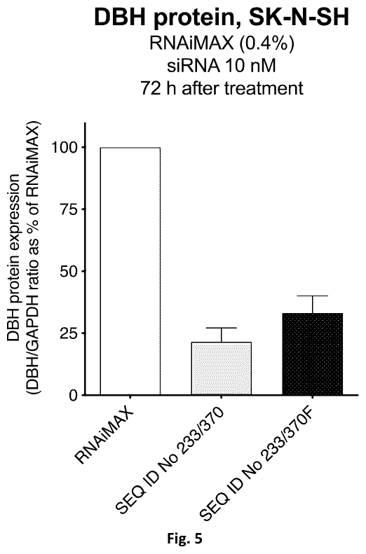

FIG. 6. Absolute and relative (dopamine/noradrenaline ratio) tissue levels of L-dihydroxyphenylalanine (L-DOPA), dopamine and noradrenaline in the eye, brain parietal cortex, brainstem and heart (left ventricle) in Wistar rats 8 h after oral administration of vehicle or nepicastat (30 mg/kg). Significantly different from corresponding control values (* P<0.05).

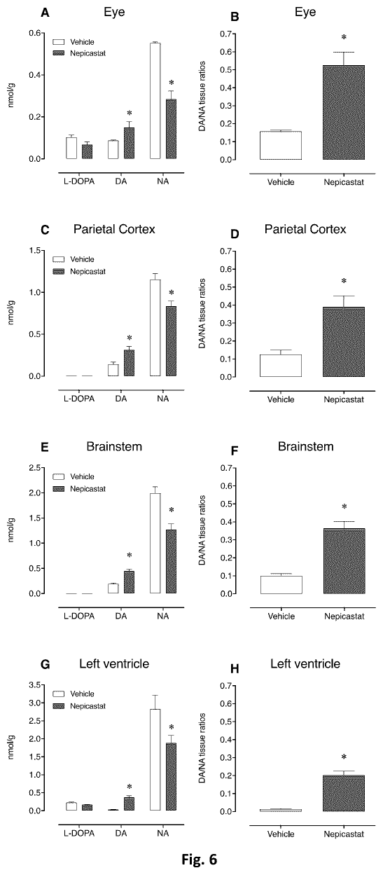

### DETAILED DESCRIPTION

The present disclosure relates to method of producing and using short interfering nucleic acids (siNAs) for preventing or reversing progressive optical neuropathy associated with the elevation of intraocular pressure due to excessive noradrenergic activation.

In an embodiment, the present disclosure relates to a method of producing and using siNAs for treating, preventing or reversing progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the siNA or vector encoding the siNA, or the medicament comprising the siNA, or vector encoding the siNA, is administered to an individual by topical eye application, subconjunctival injection, intravitreal injection, retrobulbar injection, intracameral injection, subtenon injection or deposition, intravenous injection, intravenous infusion.

In an embodiment, the present disclosure relates to an in vitro method of inhibiting the expression of dopamine-beta-hydroxylase gene in a cell comprising contacting the cell with siNA that inhibits dopamine-beta-hydroxylase gene expression.

In an embodiment, said siRNA comprises a sense dopamine-beta-hydroxylase nucleic acid and an anti-sense dopamine-beta-hydroxylase nucleic acid, wherein the sense dopamine-beta-hydroxylase nucleic acid is substantially identical to a target sequence contained within dopamine-beta-hydroxylase mRNA and the anti-sense dopamine-beta-hydroxylase nucleic acid is complementary to the sense dopamine-beta-hydroxylase nucleic acid.

In an embodiment, the present disclosure also relates to an in vitro method of inhibiting the expression of the dopamine-beta-hydroxylase gene in a cell comprising contacting the cell with a vector encoding a siRNA that inhibits dopamine-beta-hydroxylase gene expression, said siRNA comprises a sense dopamine-beta-hydroxylase nucleic acid and an anti-sense dopamine-beta-hydroxylase nucleic acid, wherein the sense dopamine-beta-hydroxylase nucleic acid is substantially identical to a target sequence contained within dopamine-beta-hydroxylase mRNA and the anti-sense dopamine-beta-hydroxylase nucleic acid is complementary to the sense dopamine-beta-hydroxylase nucleic acid.

In an embodiment, the expression of the gene is inhibited by introduction of a double stranded ribonucleic acid (dsRNA) molecule into the cell in an amount sufficient to inhibit expression of the dopamine-beta-hydroxylase gene.

In an embodiment, the siRNAs used in the disclosure cause RNAi-mediated degradation of dopamine-beta-hydroxylase mRNA such that the protein product of the dopamine-beta-hydroxylase gene is not produced or is produced in reduced amounts.

In an embodiment, the siRNAs of the present disclosure can be used to alter gene expression in a cell in which expression of dopamine-beta-hydroxylase is initiated, e.g., as a result of excessive noradrenergic activation. Binding of the siRNA to a dopamine-beta-hydroxylase mRNA transcript in a cell results in a reduction in dopamine-beta-hydroxylase protein production by the cell.

The term “siRNA” is used to mean a double stranded RNA molecule which prevents translation of a target mRNA. Standard techniques of introducing siRNA into the cell are used, including those in which DNA is a template from which RNA is transcribed. The siRNA that inhibits dopamine-beta-hydroxylase gene expression includes a sense dopamine-beta-hydroxylase nucleic acid sequence and an antisense dopamine-beta-hydroxylase nucleic acid sequence. The siRNA may be constructed such that a single transcript has both the sense and complementary antisense sequences from the target gene, e.g., in the form of a hairpin.

In an embodiment, the siRNA preferably comprises short double-stranded RNA that is targeted to the target mRNA, i.e., dopamine-beta-hydroxylase protein from dopamine-beta-hydroxylase mRNA. The siRNA comprises a sense RNA strand and a complementary antisense RNA strand annealed together by standard Watson-Crick base-pairing interactions (hereinafter “base-paired”). The sense strand comprises a nucleic acid sequence which is substantially identical to a target sequence contained within the dopamine-beta-hydroxylase mRNA.

The terms “sense/antisense sequences” and “sense/antisense strands” are used interchangeable herein to refer to the parts of the siRNA of the present disclosure that are substantially identical (sense) to the target dopamine-beta-hydroxylase mRNA sequence or substantially complementary (antisense) to the target dopamine-beta-hydroxylase mRNA sequence.

As used herein, a nucleic acid sequence “substantially identical” to a target sequence contained within the target mRNA is a nucleic acid sequence which is identical to the target sequence, or which differs from the target sequence by one or more nucleotides. Preferably, the substantially identical sequence is identical to the target sequence or differs from the target sequence by one, two or three nucleotides, more preferably by one or two nucleotides and most preferably by only 1 nucleotide. Sense strands which comprise nucleic acid sequences substantially identical to a target sequence are characterized in that siRNA comprising such a sense strand induces RNAi-mediated degradation of mRNA containing the target sequence. For example, an siRNA of the disclosure can comprise a sense strand comprising a nucleic acid sequence which differs from a target sequence by one, two, three or more nucleotides, as long as RNAi-mediated degradation of the target mRNA is induced by the siRNA.

The sense and antisense strands of the siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The siRNA can also contain alterations, substitutions or modifications of one or more ribonucleotide bases. For example, the present siRNA can be altered, substituted or modified to contain one or more, preferably 0, 1, 2 or 3, deoxyribonucleotide bases. Preferably, the siRNA does not contain any deoxyribonucleotide bases.

In an embodiment, the siRNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more, preferably 0, 1, 2 or 3, nucleotides in the siRNA with deoxyribonucleotides.

Degradation can be delayed or avoided by a wide variety of chemical modifications that include alterations in the nucleobases, sugars and the phosphate ester backbone of the siRNAs. All of these chemically modified siRNAs are still able to induce siRNA-mediated gene silencing provided that the modifications were absent in specific regions of the siRNA and included to a limited extent. In general, backbone modifications cause a small loss in binding affinity, but offer nuclease resistance. Phosphorothioate (PS)- or boranophosphate (BS)-modified siRNAs have substantial nuclease resistance. Silencing by siRNA duplexes is also compatible with some types of 2′-sugar modifications: 2′-H,2′-O-methyl, 2′-O-methoxyethyl, 2′-fluoro (2′-F), locked nucleic acid (LNA) and ethylene-bridge nucleic acid (ENA). Suitable chemical modifications are well known to those skilled in the art.

In an embodiment, the siRNA used in the present disclosure is a double-stranded molecule comprises a sense strand and an antisense strand, wherein the sense strand comprises a ribonucleotide sequence corresponding to dopamine-beta-hydroxylase protein target sequence, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the dopamine-beta-hydroxylase gene, inhibits expression of the said gene. As indicated further below, said dopamine-beta-hydroxylase target sequence preferably comprises at least about contiguous, more preferably 19 to 25, and most preferably about 19 to 21 contiguous nucleotides selected from the group consisting of from SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132.

In an embodiment, the siRNA used in the present disclosure can be obtained using a number of techniques known to those of skill in the art. For example, the siRNA can be chemically synthesized or recombinantly produced using methods known in the art, such as the Drosophila in vitro system described in U.S. published application 2002/0086356, the entire disclosure of which is herein incorporated by reference. The siRNA may be chemically synthesized using appropriately protected ribonucleoside phosphoramidites and a conventional DNA/RNA synthesizer.

In an embodiment, the siRNA can be synthesized as two separate, complementary RNA molecules, or as a single RNA molecule with two complementary regions. Commercial suppliers of synthetic RNA molecules or synthesis reagents include Biospring (Frankfurt, Germany), ChemGenes (Ashland, Mass., USA), Dharmacon Research (Lafayette, Colo., USA), Glen Research (Sterling, Va., USA), Proligo (Hamburg, Germany), Sigma-Aldrich (St. Louis, MO USA) and Thermo Fisher Scientific (Waltham, MA USA).

In an embodiment, the siRNA can also be expressed from recombinant circular or linear DNA vectors using any suitable promoter. Suitable promoters for expressing siRNA from a vector include, for example, the U6 or H1 RNA pol III promoter sequences and the cytomegalovirus promoter. Selection of other suitable promoters is within the skill in the art. The vector can also comprise inducible or regulable promoters for expression of the siRNA in a particular tissue or in a particular intracellular environment.

In an embodiment, the siRNA expressed from a vector can either be isolated from cultured cell expression systems by standard techniques, or can be expressed intracellularly. The vector can be used to deliver the siRNA to cells in vivo, e.g., by intracellularly expressing the siRNA in vivo. siRNA can be expressed from a vector either as two separate, complementary RNA molecules, or as a single RNA molecule with two complementary regions. Selection of vectors suitable for expressing the siRNA, methods for inserting nucleic acid sequences for expressing the siRNA into the vector, and methods of delivering the vector to the cells of interest are well known to those skilled in the art.

In an embodiment, the siRNA can also be expressed from a vector intracellularly in vivo.

As used herein, the term “vector” means any nucleic acid- and/or viral-based technique used to deliver a desired nucleic acid. Any vector capable of accepting the coding sequences for the siRNA molecule(s) to be expressed can be used, including plasmids, cosmids, naked DNA, optionally condensed with a condensing agent, and viral vectors. Suitable viral vectors include vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate. When the vector is a lentiviral vector it is preferably pseudotyped with surface proteins from vesicular stomatitis virus, rabies virus, Ebola virus or Mokola virus.

In an embodiment, vectors are produced, for example, by cloning the dopamine-beta-hydroxylase target sequence into an expression vector so that operatively-linked regulatory sequences flank the dopamine-beta-hydroxylase sequence in a manner that allows for expression (by transcription of the DNA molecule) of both strands (Lee et al., 2002). An RNA molecule that is antisense to dopamine-beta-hydroxylase mRNA is transcribed by a first promoter (e.g., a promoter sequence 3′ of the cloned DNA) and an RNA molecule that is the sense strand for the dopamine-beta-hydroxylase mRNA is transcribed by a second promoter (e. g., a promoter sequence 5′ of the cloned DNA). The sense and antisense strands hybridize in vivo to generate siRNA constructs for silencing of the dopamine-beta-hydroxylase gene. Alternatively, two vectors are utilized to create the sense and anti-sense strands of a siRNA construct. Cloned dopamine-beta-hydroxylase can encode a construct having secondary structure, e. g., hairpins, wherein a single transcript has both the sense and complementary antisense sequences from the target gene. Such a transcript encoding a construct having secondary structure, will preferably comprises a single-stranded ribonucleotide sequence (loop sequence) linking said sense strand and said antisense strand.

In an embodiment, the siRNA is preferably isolated.

As used herein, “isolated” means synthetic, or altered or removed from the natural state through human intervention. For example, a siRNA naturally present in a living animal is not “isolated,” but a synthetic siRNA, or a siRNA partially or completely separated from the coexisting materials of its natural state is “isolated.” An isolated siRNA can exist in substantially purified form, or can exist in a non-native environment such as, for example, a cell into which the siRNA has been delivered. By way of example, siRNA which are produced inside a cell by natural processes, but which are produced from an “isolated” precursor molecule, are themselves “isolated” molecules. Thus, an isolated dsRNA can be introduced into a target cell, where it is processed by the Dicer protein (or its equivalent) into isolated siRNA.

As used herein, “inhibit” means that the activity of the dopamine-beta-hydroxylase gene expression product or level of the dopamine-beta-hydroxylase gene expression product is reduced below that observed in the absence of the siRNA molecule of the disclosure. The inhibition with a siRNA molecule preferably is significantly below that level observed in the presence of an inactive or attenuated molecule that is unable to mediate an RNAi response. Inhibition of gene expression with the siRNA molecule is preferably significantly greater in the presence of the siRNA molecule than in its absence. Preferably, the siRNA inhibits the level of dopamine-beta-hydroxylase gene expression by at least 10%, more preferably at least 50% and most preferably at least 75%.

Preferably the siRNA molecule inhibits dopamine-beta-hydroxylase gene expression so that the protein product of the dopamine-beta-hydroxylase gene is not produced or is produced in reduced amounts. By inhibiting dopamine-beta-hydroxylase expression for is meant that the treated cell produces at a lower rate or has decreased the dopamine-beta-hydroxylase protein that allows the prevention or reversion of progressive optical neuropathy associated with the elevation of intraocular pressure due to excessive noradrenergic activation. The dopamine-beta-hydroxylase is measured by mRNA or protein assays known in the art.

As used herein, an “isolated nucleic acid” is a nucleic acid removed from its original environment (e. g., the natural environment if naturally occurring) and thus, synthetically altered from its natural state. In the present disclosure, isolated nucleic acid includes DNA, RNA, and derivatives thereof. When the isolated nucleic acid is RNA or derivatives thereof, base “T” should be replaced with “U” in the nucleotide sequences.

As used herein, the term “complementary” refers to Watson-Crick or Hoogsteen base pairing between nucleotides units of a polynucleotide, and the term “binding” means the physical or chemical interaction between two polypeptides or compounds or associated polypeptides or compounds or combinations thereof.

As used herein, the phrase “highly conserved sequence region” means a nucleotide sequence of one or more regions in a target gene does not vary significantly from one generation to the other or from one biological system to the other.

As used herein, the term “complementarity” or “complementary” means that a nucleic acid can form hydrogen bond(s) with another nucleic acid sequence by either traditional Watson-Crick or other non-traditional types of interaction. In reference to the present disclosure, the binding free energy for a siRNA molecule with its complementary sequence is sufficient to allow the relevant function of the nucleic acid to proceed, e.g., RNAi activity. For example, the degree of complementarity between the sense and antisense strand of the siRNA molecule can be the same or different from the degree of complementarity between the antisense strand of the siRNA and the target RNA sequence.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. Preferably the term “complementarity” or “complementary” means that at least 90%, more preferably at least 95% and most preferably 100% of residues in a first nucleic acid sense can form hydrogen binds with a second nucleic acid sequence.

Complementary nucleic acid sequences hybridize under appropriate conditions to form stable duplexes containing few (one or two) or no mismatches. Furthermore, the sense strand and antisense strand of the siRNA can form a double stranded nucleotide or hairpin loop structure by the hybridization.

In an embodiment, such duplexes contain no more than 1 mismatch for every 10 matches.

In an embodiment, the sense and antisense strands of the duplex are fully complementary, i.e., the duplexes contain no mismatches.

As used herein, the term “cell” is defined using its usual biological sense. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell. Preferably the cell is in eye, eye cornea, eye ciliary body, eye trabecular mesh, eye retina, brain, colon, head and neck, kidney, liver, lung, or lymph.

As used herein, the term “RNA” means a molecule comprising at least one ribonucleotide residue. By “ribonucleotide” is meant a nucleotide with a hydroxyl group at the 2′ position of a beta-D-ribo-furanose moiety. The term includes double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogues of naturally-occurring RNA. Preferably the term “RNA” consists of ribonucleotide residues only.

As used herein, the term “organism” refers to any living entity comprised of at least one cell. A living organism can be as simple as, for example, a single eukaryotic cell or as complex as a mammal, including a human being.

As used herein, the term “subject” means an organism, which is a donor or recipient of explanted cells or the cells themselves. “Subject” also refers to an organism to which the nucleic acid molecules of the disclosure can be administered. The subject is preferably a mammal, e.g., a human, non-human primate, mouse, rat, dog, cat, horse, or cow. Most preferably the subject is a human.

As used herein, the term “biological sample” refers to any sample containing polynucleotides. The sample may be a tissue or cell sample, or a body fluid containing polynucleotides (e.g., blood, mucus, lymphatic fluid, synovial fluid, cerebrospinal fluid, saliva, amniotic fluid, amniotic cord blood, urine, vaginal fluid and semen). The sample may be a homogenate, lysate, extract, cell culture or tissue culture prepared from a whole organism or a subset of its cells, tissues or component parts, or a fraction or portion thereof. Lastly, the sample may be a medium, such as a nutrient broth or gel in which an organism, or cells of an organism, have been propagated, wherein the sample contains polynucleotides.

In an embodiment, the disclosure relates to methods of producing and using short interfering nucleic acids (siNAs) for preventing or reversing progressive optical neuropathy associated with the elevation of intraocular pressure due to excessive noradrenergic activation.

In an embodiment, this disclosure relates to a method of producing and using siNAs for preventing or reversing progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma. The cell may be further contacted with a transfection-enhancing agent to enhance delivery of the siRNA or siRNA encoding vector to the cell. Depending on the specific method of the present disclosure, the cell may be provided in vitro, in vivo or ex vivo.

Sequence information regarding the dopamine-beta-hydroxylase protein gene (GenBank accession NM_000787.4) was extracted from the NCBI Entrez nucleotide database. Up to 137 mRNA segments were identified. See for example, U.S. Pat. No. 6,506,559, and Elbashir et al., 2001, herein incorporated by reference in its entirety.

Selection of siRNA target sites can be performed as follows:
i) Beginning with the ATG start codon of the transcript, scan downstream for AA dinucleotide sequences. Record the occurrence of each AA and the 3′ adjacent 19 nucleotides as potential siRNA target sites. Tuschl et al. (Tuschl, Sharp & Bartel, 1998) recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
ii) Compare the potential target sites to the appropriate genome database (human, mouse, rat, etc.) and eliminate from consideration any target sequences with significant homology to other coding sequences. We suggest using BLAST, which can be found on the NCBI server at: www.ncbi.nlm.nih.gov/BLAST/
iii) Select qualifying target sequences (i.e., sequences having over 45% GC content) for synthesis.

In an embodiment, the length of the sense nucleic acid is at least 10 nucleotides and may be as long as the naturally-occurring dopamine-beta-hydroxylase transcript.

In an embodiment, the length of the sense nucleic acid is preferably less than 75 nucleotides in length, preferably 50 nucleotides in length, more preferably 25 nucleotides in length.

In an embodiment, the length of the sense nucleic acid is at least 19 nucleotides, preferably, 19-25 nucleotides in length.

Examples of dopamine-beta-hydroxylase target siRNA sense nucleic acids of the present disclosure which inhibit dopamine-beta-hydroxylase expression in mammalian cells include oligonucleotides comprising any one of the following target sequences of the dopamine-beta-hydroxylase gene: SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132.

One hundred and thirty-seven sequences, which set forth the sequence for one strand of the double stranded is RNA, were identified and isolated for dopamine-beta-hydroxylase (Table 1).

TABLE 1
5′ sense dopamine-beta-hydroxylase target protein.

| SEQ ID No | 5′ DNA sense |
|---|---|
| SEQ ID No 1 | GTCCCTGGAGCTCTCATGGAA |
| SEQ ID No 2 | AATGTCAGCTACACCCAGGAG |
| SEQ ID No 3 | GCTCCTGGTGCGGAGGCTCAA |
| SEQ ID No 4 | AAGGCTGGCGTCCTGTTTGGG |
| SEQ ID No 5 | TGTCCGACCGTGGCGAGCTTGAGAA |
| SEQ ID No 6 | TCCGACCGTGGCGAGCTTGAGAA |
| SEQ ID No 7 | CGACCGTGGCGAGCTTGAGAA |
| SEQ ID No 8 | ACCGTGGCGAGCTTGAGAA |
| SEQ ID No 9 | AACGCAGATCTCGTGGTGCTC |
| SEQ ID No 10 | GGACGCCTGGAGTGACCAGAA |
| SEQ ID No 11 | AAGGGGCAGATCCACCTGGAT |
| SEQ ID No 12 | CAGGTGCAGAGGACCCCAGAA |
| SEQ ID No 13 | AAGGCCTGACCCTGCTTTTCA |
| SEQ ID No 14 | AAGGCCTGACCCTGCTTTT |
| SEQ ID No 15 | AAGGCCTGACCCTGCTTTTCAAG |
| SEQ ID No 16 | AAGGCCTGACCCTGCTTTTCAAGAG |
| SEQ ID No 17 | AGGCCTGACCCTGCTTTTCAA |
| SEQ ID No 18 | AAGAGGCCCTTTGGCACCTGC |
| SEQ ID No 19 | CTTTGGCACCTGCGACCCCAA |
| SEQ ID No 20 | CCCAAGGATTACCTCATTGAA |
| SEQ ID No 21 | AAGGATTACCTCATTGAAGAC |
| SEQ ID No 22 | AAGACGGCACTGTCCACTTGG |
| SEQ ID No 23 | CCGGTCACTGGAGGCCATCAA |
| SEQ ID No 24 | AACGGCTCGGGCCTGCAGATG |
| SEQ ID No 25 | GCAGAGGGTGCAGCTCCTGAA |
| SEQ ID No 26 | GGTGCAGCTCCTGAAGCCCAA |
| SEQ ID No 27 | CTGAAGCCCAATATCCCCGAA |
| SEQ ID No 28 | AAGCCCAATATCCCCGAACCG |
| SEQ ID No 29 | AATATCCCCGAACCGGAGTTG |
| SEQ ID No 30 | AACCGGAGTTGCCCTCAGACG |
| SEQ ID No 31 | GCGTGCACCATGGAGGTCCAA |
| SEQ ID No 32 | CATGGAGGTCCAAGCTCCCAA |
| SEQ ID No 33 | AAGCTCCCAATATCCAGATCC |
| SEQ ID No 34 | AATATCCAGATCCCCAGCCAG |
| SEQ ID No 35 | AGACCACGTACTGGTGCTACATTAA |
| SEQ ID No 36 | ACCACGTACTGGTGCTACATTAA |
| SEQ ID No 37 | CACGTACTGGTGCTACATTAA |
| SEQ ID No 38 | CGTACTGGTGCTACATTAA |
| SEQ ID No 39 | GCTACATTAAGGAGCTTCCAA |
| SEQ ID No 40 | CTACATTAAGGAGCTTCCAAA |
| SEQ ID No 41 | AAGGAGCTTCCAAAGGGCTTC |
| SEQ ID No 42 | AAAGGGCTTCTCTCGGCACCA |
| SEQ ID No 43 | AAGGGCTTCTCTCGGCACCAC |
| SEQ ID No 44 | GCTTCTCTCGGCACCACATTATCAA |
| SEQ ID No 45 | TTCTCTCGGCACCACATTATCAA |
| SEQ ID No 46 | CTCTCGGCACCACATTATCAA |
| SEQ ID No 47 | CTCGGCACCACATTATCAA |
| SEQ ID No 48 | AAGTACGAGCCCATCGTCACC |
| SEQ ID No 49 | GTACGAGCCCATCGTCACCAA |
| SEQ ID No 50 | GCCCATCGTCACCAAGGGCAA |
| SEQ ID No 51 | AAGGGCAATGAGGCCCTTGTC |
| SEQ ID No 52 | AATGAGGCCCTTGTCCACCAC |
| SEQ ID No 53 | GCCCTTGTCCACCACATGGAA |
| SEQ ID No 54 | AAGTCTTCCAGTGCGCCCCCG |
| SEQ ID No 55 | CAGCGGGCCCTGCGACTCCAA |
| SEQ ID No 56 | CGGGCCCTGCGACTCCAAGATGAAA |
| SEQ ID No 57 | GGCCCTGCGACTCCAAGATGAAA |
| SEQ ID No 58 | GCCCTGCGACTCCAAGATGAA |
| SEQ ID No 59 | CCCTGCGACTCCAAGATGAAA |
| SEQ ID No 60 | CTGCGACTCCAAGATGAAA |
| SEQ ID No 61 | AAGATGAAACCCGACCGCCTC |
| SEQ ID No 62 | GATGAAACCCGACCGCCTCAA |
| SEQ ID No 63 | AAACCCGACCGCCTCAACTAC |
| SEQ ID No 64 | AACCCGACCGCCTCAACTACT |
| SEQ ID No 65 | AACTACTGCCGCCACGTGCTG |
| SEQ ID No 66 | CGCCTGGGCCCTGGGTGCCAA |
| SEQ ID No 67 | AAGGCATTTTACTACCCAGAG |
| SEQ ID No 68 | GCATTTTACTACCCAGAGGAA |
| SEQ ID No 69 | AAGCCGGCCTTGCCTTCGGGG |
| SEQ ID No 70 | TCCAGATATCTCCGCCTGGAA |
| SEQ ID No 71 | CCTGGAAGTTCACTACCACAA |
| SEQ ID No 72 | AAGTTCACTACCACAACCCAC |
| SEQ ID No 73 | CACAACCCACTGGTGATAGAA |
| SEQ ID No 74 | AACCCACTGGTGATAGAAGGA |
| SEQ ID No 75 | CACTGGTGATAGAAGGACGAA |
| SEQ ID No 76 | ACTGGTGATAGAAGGACGAAA |
| SEQ ID No 77 | AAGGACGAAACGACTCCTCAG |
| SEQ ID No 78 | AAACGACTCCTCAGGCATCCG |
| SEQ ID No 79 | AACGACTCCTCAGGCATCCGC |
| SEQ ID No 80 | CCGCTTGTACTACACAGCCAA |
| SEQ ID No 81 | AGCCAAGCTGCGGCGCTTCAA |
| SEQ ID No 82 | AAGCTGCGGCGCTTCAACGCG |
| SEQ ID No 83 | AACGCGGGGATCATGGAGCTG |
| SEQ ID No 84 | CACTGGCTACTGCACGGACAA |
| SEQ ID No 85 | AAGTGCACCCAGCTGGCACTG |
| SEQ ID No 86 | ACACACACCTGACTGGGAGAA |
| SEQ ID No 87 | CACACACCTGACTGGGAGAAA |
| SEQ ID No 88 | AAAGGTGGTCACAGTGCTGGT |
| SEQ ID No 89 | AAGGTGGTCACAGTGCTGGTC |
| SEQ ID No 90 | CCGGGAGTGGGAGATCGTGAA |
| SEQ ID No 91 | GGAGATCGTGAACCAGGACAA |
| SEQ ID No 92 | AACCAGGACAATCACTACAGC |
| SEQ ID No 93 | AATCACTACAGCCCTCACTTC |
| SEQ ID No 94 | ACTTCCAGGAGATCCGCATGTTGAA |
| SEQ ID No 95 | TTCCAGGAGATCCGCATGTTGAA |
| SEQ ID No 96 | CCAGGAGATCCGCATGTTGAA |
| SEQ ID No 97 | AGGAGATCCGCATGTTGAA |
| SEQ ID No 98 | GGAGATCCGCATGTTGAAGAA |
| SEQ ID No 99 | AAGAAGGTCGTGTCGGTCCAT |
| SEQ ID No 100 | AAGGTCGTGTCGGTCCATCCG |
| SEQ ID No 101 | CATCACCTCCTGCACGTACAA |
| SEQ ID No 102 | TCCTGCACGTACAACACAGAA |
| SEQ ID No 103 | AACACAGAAGACCGGGAGCTG |
| SEQ ID No 104 | AAGACCGGGAGCTGGCCACAG |
| SEQ ID No 105 | CCTGGAGGAGATGTGTGTCAA |
| SEQ ID No 106 | AACTACGTGCACTACTACCCC |
| SEQ ID No 107 | GACGCAGCTGGAGCTCTGCAA |
| SEQ ID No 108 | AAGAGCGCTGTGGACGCCGGC |
| SEQ ID No 109 | GGACGCCGGCTTCCTGCAGAA |
| SEQ ID No 110 | GAAGTACTTCCACCTCATCAA |
| SEQ ID No 111 | AAGTACTTCCACCTCATCAAC |
| SEQ ID No 112 | CCACCTCATCAACAGGTTCAA |
| SEQ ID No 113 | CCTCATCAACAGGTTCAACAA |
| SEQ ID No 114 | AACAGGTTCAACAACGAGGAT |
| SEQ ID No 115 | AACAACGAGGATGTCTGCACC |
| SEQ ID No 116 | AACGAGGATGTCTGCACCTGC |
| SEQ ID No 117 | GTTCACCTCTGTTCCCTGGAA |
| SEQ ID No 118 | TGTTCCCTGGAACTCCTTCAA |
| SEQ ID No 119 | AACTCCTTCAACCGCGACGTA |
| SEQ ID No 120 | CTTCAACCGCGACGTACTGAA |
| SEQ ID No 121 | AACCGCGACGTACTGAAGGCC |
| SEQ ID No 122 | AAGGCCCTGTACAGCTTCGCG |
| SEQ ID No 123 | GCCCATCTCCATGCACTGCAA |
| SEQ ID No 124 | CATCTCCATGCACTGCAACAA |
| SEQ ID No 125 | AACAAGTCCTCAGCCGTCCGC |
| SEQ ID No 126 | AAGTCCTCAGCCGTCCGCTTC |
| SEQ ID No 127 | GCCGTCCGCTTCCAGGGTGAA |
| SEQ ID No 128 | CCGCTTCCAGGGTGAATGGAA |
| SEQ ID No 129 | AATGGAACCTGCAGCCCCTGC |
| SEQ ID No 130 | GAACCTGCAGCCCCTGCCCAA |
| SEQ ID No 131 | AACCTGCAGCCCCTGCCCAAG |
| SEQ ID No 132 | AAGGTCATCTCCACACTGGAA |
| SEQ ID No 133 | AAGAGCCCACCCCACAGTGCC |
| SEQ ID No 134 | GCCCCACCAGCCAGGGCCGAA |
| SEQ ID No 135 | AAGCCCTGCTGGCCCCACCGT |
| SEQ ID No 136 | TGTCAGCATTGGTGGGGGCAA |
| SEQ ID No 137 | GTCAGCATTGGTGGGGGCAAA |

The dopamine-beta-hydroxylase gene specificity was confirmed by searching NCBI BlastN database. The siRNAs were chemically synthesized.

All of the purified siRNA duplexes were complexed with lipofectamine and added to the cells for up to 12 h in serum-free medium. Thereafter, cells were cultured for 24-96 h in serum-supplemented medium, which was replaced by serum-free medium 24 h before the experiments. A scrambled negative siRNA duplex was used as control.

The dopamine-beta-hydroxylase-siRNA is directed to a single target dopamine-beta-hydroxylase gene sequence. Alternatively, the siRNA is directed to multiple target dopamine-beta-hydroxylase gene sequences. For example, the composition contains dopamine-beta-hydroxylase-siRNA directed to two, three, four, five or more dopamine-beta-hydroxylase target sequences. By dopamine-beta-hydroxylase target sequence is meant a nucleotide sequence that is identical to a portion of the dopamine-beta-hydroxylase gene. The target sequence can include the 5′ untranslated (UT) region, the open reading frame (ORF) or the 3′ untranslated region of the dopamine-beta-hydroxylase gene. Alternatively, the siRNA is a nucleic acid sequence complementary to an upstream or downstream modulator of dopamine-beta-hydroxylase gene expression. Examples of upstream and downstream modulators include a transcription factor that binds the dopamine-beta-hydroxylase gene promoter, a kinase or phosphatase that interacts with the dopamine-beta-hydroxylase polypeptide, a dopamine-beta-hydroxylase promoter or enhance.

In an embodiment, the dopamine-beta-hydroxylase-siRNA hybridize to target mRNA and decrease or inhibit production of the dopamine-beta-hydroxylase polypeptide product encoded by the dopamine-beta-hydroxylase gene by associating with the normally single-stranded mRNA transcript, thereby interfering with translation and thus, expression of the protein.

Exemplary nucleic acid sequence for the production of dopamine-beta-hydroxylase-siRNA include the sequences of nucleotides SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132 as the target sequence. In a further embodiment, in order to enhance the inhibition activity of the siRNA, nucleotide “U” can be added to 3′ end of the antisense strand of the target sequence. Preferably at least 2, more preferably 2 to 10, and most preferably 2 to 5 U's are added. The added U's form single strand at the 3′ end of the antisense strand of the siRNA.

In an embodiment, the dopamine-beta-hydroxylase-siRNA is directly introduced into the cells in a form that is capable of binding to the mRNA transcripts. Alternatively, a vector encoding the dopamine-beta-hydroxylase-siRNA can be introduced into the cells.

A loop sequence consisting of an arbitrary nucleotide sequence can be located between the sense and antisense sequence in order to form a hairpin loop structure.

In an embodiment, the present disclosure also relates to siRNA having the general formula 5′-[A]-[B]-[A′]-3′, wherein [A] is a ribonucleotide sequence corresponding to a target sequence of the spike (S) glycoprotein gene.

In an embodiment, preferably [A] is a sequence selected from the group consisting of nucleotides SEQ ID No 5, SEQ ID No 7, SEQ ID No 8, SEQ ID No 13, SEQ ID No 14, SEQ ID No 16, SEQ ID No 17, SEQ ID No 20, SEQ ID No 21, SEQ ID No 35 SEQ ID No 37, SEQ ID No 38, SEQ ID No 44, SEQ ID No 46, SEQ ID No 47, SEQ ID No 56, SEQ ID No 58, SEQ ID No 59, SEQ ID No 60, SEQ ID No 63, SEQ ID No 64, SEQ ID No 73, SEQ ID No 74, SEQ ID No 94, SEQ ID No 96, S SEQ ID No 97, EQ ID No 98, SEQ ID No 102, SEQ ID No 111, SEQ ID No 113, SEQ ID No 128 and SEQ ID No 132; [B] is a ribonucleotide sequence consisting of 3 to 23 nucleotides; and [A′] is a ribonucleotide sequence consisting of the complementary sequence of [A]. The region [A] hybridizes to [A′], and then a loop consisting of region [B] is formed. The loop sequence may be preferably 3 to 23 nucleotides in length. Suitable loop sequences are described at http://www.ambion.com/techlib/tb/tb_506.html. Furthermore, loop sequence consisting of 23 nucleotides also provides active siRNA (Jacque et al., 2002).

In an embodiment, the 5′ sense siRNA sequences against dopamine-beta-hydroxylase target sequences were identified. The 5′ anti-sense siRNA sequences against dopamine-beta-hydroxylase were then designed and produced. Sense and anti-sense siRNA sequences have a length of 19 to 25 nucleotides. Table 2 shows 5′ sense and anti-sense siRNA sequences against dopamine-beta-hydroxylase. siRNA sequences have a length of 19 to 25 nucleotides.

TABLE 2
5′ sense and anti-sense siRNA sequences of dopamine-beta-hydroxylase-19 to 25
nucleotides.

| SEQ ID No | 5′ RNA sense | SEQ ID No | 5 RNA antisense |
|---|---|---|---|
| SEQ ID No 138 | GUCCCUGGAGCUCUCAUGGAA | SEQ ID No 275 | UUCCAUGAGAGCUCCAGGGAC |
| SEQ ID No 139 | AAUGUCAGCUACACCCAGGAG | SEQ ID No 276 | CUCCUGGGUGUAGCUGACAUU |
| SEQ ID No 140 | GCUCCUGGUGCGGAGGCUCAA | SEQ ID No 277 | UUGAGCCUCCGCACCAGGAGC |
| SEQ ID No 141 | AAGGCUGGCGUCCUGUUUGGG | SEQ ID No 278 | CCCAAACAGGACGCCAGCCUU |
| SEQ ID No 142 | UGUCCGACCGUGGCGAGCUUGAGAA | SEQ ID No 279 | UUCUCAAGCUCGCCACGGUCGGACA |
| SEQ ID No 143 | UCCGACCGUGGCGAGCUUGAGAA | SEQ ID No 280 | UUCUCAAGCUCGCCACGGUCGGA |
| SEQ ID No 144 | CGACCGUGGCGAGCUUGAGAA | SEQ ID No 281 | UUCUCAAGCUCGCCACGGUCG |
| SEQ ID No 145 | ACCGUGGCGAGCUUGAGAA | SEQ ID No 282 | UUCUCAAGCUCGCCACGGU |
| SEQ ID No 146 | AACGCAGAUCUCGUGGUGCUC | SEQ ID No 283 | GAGCACCACGAGAUCUGCGUU |
| SEQ ID No 147 | GGACGCCUGGAGUGACCAGAA | SEQ ID No 284 | UUCUGGUCACUCCAGGCGUCC |
| SEQ ID No 148 | AAGGGGCAGAUCCACCUGGAU | SEQ ID No 285 | AUCCAGGUGGAUCUGCCCCUU |
| SEQ ID No 149 | CAGGUGCAGAGGACCCCAGAA | SEQ ID No 286 | UUCUGGGGUCCUCUGCACCUG |
| SEQ ID No 150 | AAGGCCUGACCCUGCUUUUCA | SEQ ID No 287 | UGAAAAGCAGGGUCAGGCCUU |
| SEQ ID No 151 | AAGGCCUGACCCUGCUUUU | SEQ ID No 288 | AAAAGCAGGGUCAGGCCUU |
| SEQ ID No 152 | AAGGCCUGACCCUGCUUUUCAAG | SEQ ID No 289 | CUUGAAAAGCAGGGUCAGGCCUU |
| SEQ ID No 153 | AAGGCCUGACCCUGCUUUUCAAGAG | SEQ ID No 290 | CUCUUGAAAAGCAGGGUCAGGCCUU |
| SEQ ID No 154 | AGGCCUGACCCUGCUUUUCAA | SEQ ID No 291 | UUGAAAAGCAGGGUCAGGCCU |
| SEQ ID No 155 | AAGAGGCCCUUUGGCACCUGC | SEQ ID No 292 | GCAGGUGCCAAAGGGCCUCUU |
| SEQ ID No 156 | CUUUGGCACCUGCGACCCCAA | SEQ ID No 293 | UUGGGGUCGCAGGUGCCAAAG |
| SEQ ID No 157 | CCCAAGGAUUACCUCAUUGAA | SEQ ID No 294 | UUCAAUGAGGUAAUCCUUGGG |
| SEQ ID No 158 | AAGGAUUACCUCAUUGAAGAC | SEQ ID No 295 | GUCUUCAAUGAGGUAAUCCUU |
| SEQ ID No 159 | AAGACGGCACUGUCCACUUGG | SEQ ID No 296 | CCAAGUGGACAGUGCCGUCUU |
| SEQ ID No 160 | CCGGUCACUGGAGGCCAUCAA | SEQ ID No 297 | UUGAUGGCCUCCAGUGACCGG |
| SEQ ID No 161 | AACGGCUCGGGCCUGCAGAUG | SEQ ID No 298 | CAUCUGCAGGCCCGAGCCGUU |
| SEQ ID No 162 | GCAGAGGGUGCAGCUCCUGAA | SEQ ID No 299 | UUCAGGAGCUGCACCCUCUGC |
| SEQ ID No 163 | GGUGCAGCUCCUGAAGCCCAA | SEQ ID No 300 | UUGGGCUUCAGGAGCUGCACC |
| SEQ ID No 164 | CUGAAGCCCAAUAUCCCCGAA | SEQ ID No 301 | UUCGGGGAUAUUGGGCUUCAG |
| SEQ ID No 165 | AAGCCCAAUAUCCCCGAACCG | SEQ ID No 302 | CGGUUCGGGGAUAUUGGGCUU |
| SEQ ID No 166 | AAUAUCCCCGAACCGGAGUUG | SEQ ID No 303 | CAACUCCGGUUCGGGGAUAUU |
| SEQ ID No 167 | AACCGGAGUUGCCCUCAGACG | SEQ ID No 304 | CGUCUGAGGGCAACUCCGGUU |
| SEQ ID No 168 | GCGUGCACCAUGGAGGUCCAA | SEQ ID No 305 | UUGGACCUCCAUGGUGCACGC |
| SEQ ID No 169 | CAUGGAGGUCCAAGCUCCCAA | SEQ ID No 306 | UUGGGAGCUUGGACCUCCAUG |
| SEQ ID No 170 | AAGCUCCCAAUAUCCAGAUCC | SEQ ID No 307 | GGAUCUGGAUAUUGGGAGCUU |
| SEQ ID No 171 | AAUAUCCAGAUCCCCAGCCAG | SEQ ID No 308 | CUGGCUGGGGAUCUGGAUAUU |
| SEQ ID No 172 | AGACCACGUACUGGUGCUACAUUAA | SEQ ID No 309 | UUAAUGUAGCACCAGUACGUGGUCU |
| SEQ ID No 173 | ACCACGUACUGGUGCUACAUUAA | SEQ ID No 310 | UUAAUGUAGCACCAGUACGUGGU |
| SEQ ID No 174 | CACGUACUGGUGCUACAUUAA | SEQ ID No 311 | UUAAUGUAGCACCAGUACGUG |
| SEQ ID No 175 | CGUACUGGUGCUACAUUAA | SEQ ID No 312 | UUAAUGUAGCACCAGUACG |
| SEQ ID No 176 | GCUACAUUAAGGAGCUUCCAA | SEQ ID No 313 | UUGGAAGCUCCUUAAUGUAGC |
| SEQ ID No 177 | CUACAUUAAGGAGCUUCCAAA | SEQ ID No 314 | UUUGGAAGCUCCUUAAUGUAG |
| SEQ ID No 178 | AAGGAGCUUCCAAAGGGCUUC | SEQ ID No 315 | GAAGCCCUUUGGAAGCUCCUU |
| SEQ ID No 179 | AAAGGGCUUCUCUCGGCACCA | SEQ ID No 316 | UGGUGCCGAGAGAAGCCCUUU |
| SEQ ID No 180 | AAGGGCUUCUCUCGGCACCAC | SEQ ID No 317 | GUGGUGCCGAGAGAAGCCCUU |
| SEQ ID No 181 | GCUUCUCUCGGCACCACAUUAUCAA | SEQ ID No 318 | UUGAUAAUGUGGUGCCGAGAGAAGC |
| SEQ ID No 182 | UUCUCUCGGCACCACAUUAUCAA | SEQ ID No 319 | UUGAUAAUGUGGUGCCGAGAGAA |
| SEQ ID No 183 | CUCUCGGCACCACAUUAUCAA | SEQ ID No 320 | UUGAUAAUGUGGUGCCGAGAG |
| SEQ ID No 184 | CUCGGCACCACAUUAUCAA | SEQ ID No 321 | UUGAUAAUGUGGUGCCGAG |
| SEQ ID No 185 | AAGUACGAGCCCAUCGUCACC | SEQ ID No 322 | GGUGACGAUGGGCUCGUACUU |
| SEQ ID No 186 | GUACGAGCCCAUCGUCACCAA | SEQ ID No 323 | UUGGUGACGAUGGGCUCGUAC |
| SEQ ID No 187 | GCCCAUCGUCACCAAGGGCAA | SEQ ID No 324 | UUGCCCUUGGUGACGAUGGGC |
| SEQ ID No 188 | AAGGGCAAUGAGGCCCUUGUC | SEQ ID No 325 | GACAAGGGCCUCAUUGCCCUU |
| SEQ ID No 189 | AAUGAGGCCCUUGUCCACCAC | SEQ ID No 326 | GUGGUGGACAAGGGCCUCAUU |
| SEQ ID No 190 | GCCCUUGUCCACCACAUGGAA | SEQ ID No 327 | UUCCAUGUGGUGGACAAGGGC |
| SEQ ID No 191 | AAGUCUUCCAGUGCGCCCCCG | SEQ ID No 328 | CGGGGGCGCACUGGAAGACUU |
| SEQ ID No 192 | CAGCGGGCCCUGCGACUCCAA | SEQ ID No 329 | UUGGAGUCGCAGGGCCCGCUG |
| SEQ ID No 193 | CGGGCCCUGCGACUCCAAGAUGAAA | SEQ ID No 330 | UUUCAUCUUGGAGUCGCAGGGCCCG |
| SEQ ID No 194 | GGCCCUGCGACUCCAAGAUGAAA | SEQ ID No 331 | UUUCAUCUUGGAGUCGCAGGGCC |
| SEQ ID No 195 | GCCCUGCGACUCCAAGAUGAA | SEQ ID No 332 | UUCAUCUUGGAGUCGCAGGGC |
| SEQ ID No 196 | CCCUGCGACUCCAAGAUGAAA | SEQ ID No 333 | UUUCAUCUUGGAGUCGCAGGG |
| SEQ ID No 197 | CUGCGACUCCAAGAUGAAA | SEQ ID No 334 | UUUCAUCUUGGAGUCGCAG |
| SEQ ID No 198 | AAGAUGAAACCCGACCGCCUC | SEQ ID No 335 | GAGGCGGUCGGGUUUCAUCUU |
| SEQ ID No 199 | GAUGAAACCCGACCGCCUCAA | SEQ ID No 336 | UUGAGGCGGUCGGGUUUCAUC |
| SEQ ID No 200 | AAACCCGACCGCCUCAACUAC | SEQ ID No 337 | GUAGUUGAGGCGGUCGGGUUU |
| SEQ ID No 201 | AACCCGACCGCCUCAACUACU | SEQ ID No 338 | AGUAGUUGAGGCGGUCGGGUU |
| SEQ ID No 202 | AACUACUGCCGCCACGUGCUG | SEQ ID No 339 | CAGCACGUGGCGGCAGUAGUU |
| SEQ ID No 203 | CGCCUGGGCCCUGGGUGCCAA | SEQ ID No 340 | UUGGCACCCAGGGCCCAGGCG |
| SEQ ID No 204 | AAGGCAUUUUACUACCCAGAG | SEQ ID No 341 | CUCUGGGUAGUAAAAUGCCUU |
| SEQ ID No 205 | GCAUUUUACUACCCAGAGGAA | SEQ ID No 342 | UUCCUCUGGGUAGUAAAAUGC |
| SEQ ID No 206 | AAGCCGGCCUUGCCUUCGGGG | SEQ ID No 343 | CCCCGAAGGCAAGGCCGGCUU |
| SEQ ID No 207 | UCCAGAUAUCUCCGCCUGGAA | SEQ ID No 344 | UUCCAGGCGGAGAUAUCUGGA |
| SEQ ID No 208 | CCUGGAAGUUCACUACCACAA | SEQ ID No 345 | UUGUGGUAGUGAACUUCCAGG |
| SEQ ID No 209 | AAGUUCACUACCACAACCCAC | SEQ ID No 346 | GUGGGUUGUGGUAGUGAACUU |
| SEQ ID No 210 | CACAACCCACUGGUGAUAGAA | SEQ ID No 347 | UUCUAUCACCAGUGGGUUGUG |
| SEQ ID No 211 | AACCCACUGGUGAUAGAAGGA | SEQ ID No 348 | UCCUUCUAUCACCAGUGGGUU |
| SEQ ID No 212 | CACUGGUGAUAGAAGGACGAA | SEQ ID No 349 | UUCGUCCUUCUAUCACCAGUG |
| SEQ ID No 213 | ACUGGUGAUAGAAGGACGAAA | SEQ ID No 350 | UUUCGUCCUUCUAUCACCAGU |
| SEQ ID No 214 | AAGGACGAAACGACUCCUCAG | SEQ ID No 351 | CUGAGGAGUCGUUUCGUCCUU |
| SEQ ID No 215 | AAACGACUCCUCAGGCAUCCG | SEQ ID No 352 | CGGAUGCCUGAGGAGUCGUUU |
| SEQ ID No 216 | AACGACUCCUCAGGCAUCCGC | SEQ ID No 353 | GCGGAUGCCUGAGGAGUCGUU |
| SEQ ID No 217 | CCGCUUGUACUACACAGCCAA | SEQ ID No 354 | UUGGCUGUGUAGUACAAGCGG |
| SEQ ID No 218 | AGCCAAGCUGCGGCGCUUCAA | SEQ ID No 355 | UUGAAGCGCCGCAGCUUGGCU |
| SEQ ID No 219 | AAGCUGCGGCGCUUCAACGCG | SEQ ID No 356 | CGCGUUGAAGCGCCGCAGCUU |
| SEQ ID No 220 | AACGCGGGGAUCAUGGAGCUG | SEQ ID No 357 | CAGCUCCAUGAUCCCCGCGUU |
| SEQ ID No 221 | CACUGGCUACUGCACGGACAA | SEQ ID No 358 | UUGUCCGUGCAGUAGCCAGUG |
| SEQ ID No 222 | AAGUGCACCCAGCUGGCACUG | SEQ ID No 359 | CAGUGCCAGCUGGGUGCACUU |
| SEQ ID No 223 | ACACACACCUGACUGGGAGAA | SEQ ID No 360 | UUCUCCCAGUCAGGUGUGUGU |
| SEQ ID No 224 | CACACACCUGACUGGGAGAAA | SEQ ID No 361 | UUUCUCCCAGUCAGGUGUGUG |
| SEQ ID No 225 | AAAGGUGGUCACAGUGCUGGU | SEQ ID No 362 | ACCAGCACUGUGACCACCUUU |
| SEQ ID No 226 | AAGGUGGUCACAGUGCUGGUC | SEQ ID No 363 | GACCAGCACUGUGACCACCUU |
| SEQ ID No 227 | CCGGGAGUGGGAGAUCGUGAA | SEQ ID No 364 | UUCACGAUCUCCCACUCCCGG |
| SEQ ID No 228 | GGAGAUCGUGAACCAGGACAA | SEQ ID No 365 | UUGUCCUGGUUCACGAUCUCC |
| SEQ ID No 229 | AACCAGGACAAUCACUACAGC | SEQ ID No 366 | GCUGUAGUGAUUGUCCUGGUU |
| SEQ ID No 230 | AAUCACUACAGCCCUCACUUC | SEQ ID No 367 | GAAGUGAGGGCUGUAGUGAUU |
| SEQ ID No 231 | ACUUCCAGGAGAUCCGCAUGUUGAA | SEQ ID No 368 | UUCAACAUGCGGAUCUCCUGGAAGU |
| SEQ ID No 232 | UUCCAGGAGAUCCGCAUGUUGAA | SEQ ID No 369 | UUCAACAUGCGGAUCUCCUGGAA |
| SEQ ID No 233 | CCAGGAGAUCCGCAUGUUGAA | SEQ ID No 370 | UUCAACAUGCGGAUCUCCUGG |
| SEQ ID No 234 | AGGAGAUCCGCAUGUUGAA | SEQ ID No 371 | UUCAACAUGCGGAUCUCCU |
| SEQ ID No 235 | GGAGAUCCGCAUGUUGAAGAA | SEQ ID No 372 | UUCUUCAACAUGCGGAUCUCC |
| SEQ ID No 236 | AAGAAGGUCGUGUCGGUCCAU | SEQ ID No 373 | AUGGACCGACACGACCUUCUU |
| SEQ ID No 237 | AAGGUCGUGUCGGUCCAUCCG | SEQ ID No 374 | CGGAUGGACCGACACGACCUU |
| SEQ ID No 238 | CAUCACCUCCUGCACGUACAA | SEQ ID No 375 | UUGUACGUGCAGGAGGUGAUG |
| SEQ ID No 239 | UCCUGCACGUACAACACAGAA | SEQ ID No 376 | UUCUGUGUUGUACGUGCAGGA |
| SEQ ID No 240 | AACACAGAAGACCGGGAGCUG | SEQ ID No 377 | CAGCUCCCGGUCUUCUGUGUU |
| SEQ ID No 241 | AAGACCGGGAGCUGGCCACAG | SEQ ID No 378 | CUGUGGCCAGCUCCCGGUCUU |
| SEQ ID No 242 | CCUGGAGGAGAUGUGUGUCAA | SEQ ID No 379 | UUGACACACAUCUCCUCCAGG |
| SEQ ID No 243 | AACUACGUGCACUACUACCCC | SEQ ID No 380 | GGGGUAGUAGUGCACGUAGUU |
| SEQ ID No 244 | GACGCAGCUGGAGCUCUGCAA | SEQ ID No 381 | UUGCAGAGCUCCAGCUGCGUC |
| SEQ ID No 245 | AAGAGCGCUGUGGACGCCGGC | SEQ ID No 382 | GCCGGCGUCCACAGCGCUCUU |
| SEQ ID No 246 | GGACGCCGGCUUCCUGCAGAA | SEQ ID No 383 | UUCUGCAGGAAGCCGGCGUCC |
| SEQ ID No 247 | GAAGUACUUCCACCUCAUCAA | SEQ ID No 384 | UUGAUGAGGUGGAAGUACUUC |
| SEQ ID No 248 | AAGUACUUCCACCUCAUCAAC | SEQ ID No 385 | GUUGAUGAGGUGGAAGUACUU |
| SEQ ID No 249 | CCACCUCAUCAACAGGUUCAA | SEQ ID No 386 | UUGAACCUGUUGAUGAGGUGG |
| SEQ ID No 250 | CCUCAUCAACAGGUUCAACAA | SEQ ID No 387 | UUGUUGAACCUGUUGAUGAGG |
| SEQ ID No 251 | AACAGGUUCAACAACGAGGAU | SEQ ID No 388 | AUCCUCGUUGUUGAACCUGUU |
| SEQ ID No 252 | AACAACGAGGAUGUCUGCACC | SEQ ID No 389 | GGUGCAGACAUCCUCGUUGUU |
| SEQ ID No 253 | AACGAGGAUGUCUGCACCUGC | SEQ ID No 390 | GCAGGUGCAGACAUCCUCGUU |
| SEQ ID No 254 | GUUCACCUCUGUUCCCUGGAA | SEQ ID No 391 | UUCCAGGGAACAGAGGUGAAC |
| SEQ ID No 255 | UGUUCCCUGGAACUCCUUCAA | SEQ ID No 392 | UUGAAGGAGUUCCAGGGAACA |
| SEQ ID No 256 | AACUCCUUCAACCGCGACGUA | SEQ ID No 393 | UACGUCGCGGUUGAAGGAGUU |
| SEQ ID No 257 | CUUCAACCGCGACGUACUGAA | SEQ ID No 394 | UUCAGUACGUCGCGGUUGAAG |
| SEQ ID No 258 | AACCGCGACGUACUGAAGGCC | SEQ ID No 395 | GGCCUUCAGUACGUCGCGGUU |
| SEQ ID No 259 | AAGGCCCUGUACAGCUUCGCG | SEQ ID No 396 | CGCGAAGCUGUACAGGGCCUU |
| SEQ ID No 260 | GCCCAUCUCCAUGCACUGCAA | SEQ ID No 397 | UUGCAGUGCAUGGAGAUGGGC |
| SEQ ID No 261 | CAUCUCCAUGCACUGCAACAA | SEQ ID No 398 | UUGUUGCAGUGCAUGGAGAUG |
| SEQ ID No 262 | AACAAGUCCUCAGCCGUCCGC | SEQ ID No 399 | GCGGACGGCUGAGGACUUGUU |
| SEQ ID No 263 | AAGUCCUCAGCCGUCCGCUUC | SEQ ID No 400 | GAAGCGGACGGCUGAGGACUU |
| SEQ ID No 264 | GCCGUCCGCUUCCAGGGUGAA | SEQ ID No 401 | UUCACCCUGGAAGCGGACGGC |
| SEQ ID No 265 | CCGCUUCCAGGGUGAAUGGAA | SEQ ID No 402 | UUCCAUUCACCCUGGAAGCGG |
| SEQ ID No 266 | AAUGGAACCUGCAGCCCCUGC | SEQ ID No 403 | GCAGGGGCUGCAGGUUCCAUU |
| SEQ ID No 267 | GAACCUGCAGCCCCUGCCCAA | SEQ ID No 404 | UUGGGCAGGGGCUGCAGGUUC |
| SEQ ID No 268 | AACCUGCAGCCCCUGCCCAAG | SEQ ID No 405 | CUUGGGCAGGGGCUGCAGGUU |
| SEQ ID No 269 | AAGGUCAUCUCCACACUGGAA | SEQ ID No 406 | UUCCAGUGUGGAGAUGACCUU |
| SEQ ID No 270 | AAGAGCCCACCCCACAGUGCC | SEQ ID No 407 | GGCACUGUGGGGUGGGCUCUU |
| SEQ ID No 271 | GCCCCACCAGCCAGGGCCGAA | SEQ ID No 408 | UUCGGCCCUGGCUGGUGGGGC |
| SEQ ID No 272 | AAGCCCUGCUGGCCCCACCGU | SEQ ID No 409 | ACGGUGGGGCCAGCAGGGCUU |
| SEQ ID No 273 | UGUCAGCAUUGGUGGGGGCAA | SEQ ID No 410 | UUGCCCCCACCAAUGCUGACA |
| SEQ ID No 274 | GUCAGCAUUGGUGGGGGCAAA | SEQ ID No 411 | UUUGCCCCCACCAAUGCUGAC |

It was surprisingly found that siRNAs targeted to certain target sequences of the dopamine-beta-hydroxylase gene are particularly effective at inhibiting dopamine-beta-hydroxylase mRNA expression, inhibiting dopamine-beta-hydroxylase expression preventing or reversing progressive optical neuropathy associated with the elevation of intraocular pressure in rats treated by ocular administration of siRNAs targeting certain sequences of the dopamine-beta-hydroxylase gene.

In an embodiment, the sense strand of the dopamine-beta-hydroxylase siRNA used in the present disclosure comprises or consists of a sequence selected from the group comprising SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265 and SEQ ID No 269.

In an embodiment, the siRNA also comprises a corresponding antisense strand comprising SEQ ID No 279, SEQ ID No 280, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

In an embodiment, the use of such an siRNA is particularly effective in inhibiting dopamine-beta-hydroxylase mRNA expression, inhibiting dopamine-beta-hydroxylase expression in rats treated by ocular administration of siRNAs targeting certain sequences of the dopamine-beta-hydroxylase gene.

In an embodiment, the present disclosure relates to a siRNA comprising a sense dopamine-beta-hydroxylase nucleic acid and an anti-sense dopamine-beta-hydroxylase nucleic acid, and the sense dopamine-beta-hydroxylase nucleic acid is substantially identical to a target sequence contained within dopamine-beta-hydroxylase mRNA and the anti-sense dopamine-beta-hydroxylase nucleic acid is complementary to the sense dopamine-beta-hydroxylase nucleic acid. The sense and antisense nucleic acids hybridize to each other to form a double-stranded molecule.

In an embodiment, the siRNA molecules of the present disclosure inhibit the expression of the dopamine-beta-hydroxylase gene when introduced into a cell expressing said gene.

In an embodiment, the siRNA molecules of the present disclosure inhibit dopamine-beta-hydroxylase expression and activity in a cell when introduced into a cell expressing dopamine-beta-hydroxylase gene.

In an embodiment, the siRNA molecules of the present disclosure decrease the expression and activity of dopamine-beta-hydroxylase in rats treated by ocular administration of siRNAs targeting certain sequences of the dopamine-beta-hydroxylase gene.

In an embodiment, the present disclosure relates to nucleic acid sequences and vectors encoding the siRNA according to the fourth aspect of the present disclosure, as well as to compositions comprising them, useful, for example, in the methods of the present disclosure. Compositions of the present disclosure may additionally comprise transfection enhancing agents. The nucleic acid sequence may be operably linked to an inducible or regulatable promoter. Suitable vectors are discussed above. Preferably the vector is an adeno-associated viral vector.

In an embodiment, the present disclosure relates to a composition comprising the siRNA of the present disclosure and additionally comprise a pharmaceutical agent for preventing or reversing progressive optical neuropathy associated with the elevation of intraocular pressure due to excessive noradrenergic activation, wherein the agent is different from the siRNA.

In an embodiment, the pharmaceutical agent is selected from the group consisting of an anti-glaucoma agent and most preferably an alpha adrenoceptor agonists, beta adrenoceptor blockers, carbonic anhydrase inhibitors, muscarinic agonists, prostaglandin analogs and rho kinase inhibitors agent and most preferably apraclonidine, brimonidine, betaxolol, levobunolol, metpranolol, timolol, acetazolamide, brinzolamide, dorzolamide, methazolamide, carbachol, pilocarpine, bimatoprost, latanoprost, tafluprost, travaprost, and netarsudil.

Non-viral delivery siRNA systems involve the creation of nucleic acid transfection reagents. Nucleic acid transfection reagents have two basic properties. First, they must interact in some manner with the nucleic acid cargo. Most often this involves electrostatic forces, which allow the formation of nucleic acid complexes. Formation of a complex ensures that the nucleic acid and transfection reagents are presented simultaneously to the cell membrane. Complexes can be divided into three classes, based on the nature of the delivery reagent: lipoplexes; polyplexes; and lipopolyplexes. Lipoplexes are formed by the interaction of anionic nucleic acids with cationic lipids, polyplexes by interaction with cationic polymers. Lipopolyplex reagents can combine the action of cationic lipids and polymers to deliver nucleic acids. Addition of histone, poly-L-lysine and protamine to some formulations of cationic lipids results in levels of delivery that are higher than either lipid or polymer alone. The combined formulations might also be less toxic. The biocompatible systems most relevant to this purpose are non-viral biodegradable nanocapsules designed especially according to the physical chemistry of nucleic acids. They have an aqueous core surrounded by a biodegradable polymeric envelope, which provides protection and transport of the siRNA into the cytosol and allow the siRNA to function efficiently in vivo.

In an embodiment, the present disclosure also relates to a cell containing the siRNA according to the present disclosure or the vector of the present disclosure. Preferably the cell is a mammalian cell, more preferably a human cell. It is further preferred that the cell is an isolated cell.

The following examples further illustrate the present disclosure in detail but are not to be construed to limit the scope thereof.

siNA molecules described in the present disclosure are tested in one or more of these examples and show to have activity and stability.

### Example 1

Cell culture: SK-N-SH cells expressing dopamine-beta-hydroxylase were maintained in a humidified atmosphere of 5% CO 2 at 37° C. Cells were grown in MEM (Sigma, St. Louis, MO) supplemented with 10% fetal bovine serum (FBS) (Gibco, UK), 100 U/mL penicillin G, 0.25 μg/mL amphotericin B, 100 μg/mL streptomycin (Gibco, UK), 25 mM sodium bicarbonate (Merck, Germany) and 25 mM N-2-hydroxyethylpiperazine-N′-2-ethanosulfonic acid (HEPES) (Sigma, St. Louis, MO). The cell culture medium was changed every 2 days, and cells reached confluence 3-4 days after initial seeding. For subculturing, cells were dissociated with 0.25% trypsin-ethylenediaminetetraacetic acid (EDTA) (Sigma, St. Louis, MO), split 1:15 or 1:20 and subcultured in a 21-cm 2 growth area (Sarstedt, Germany).

### Example 2

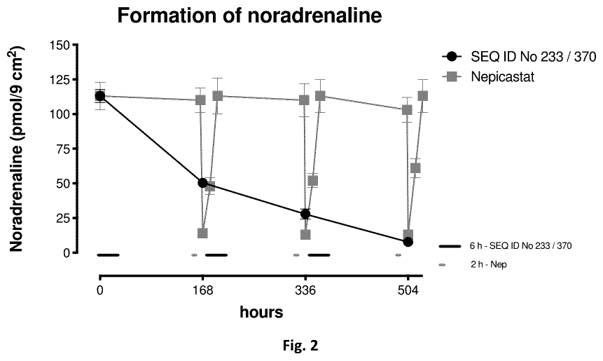

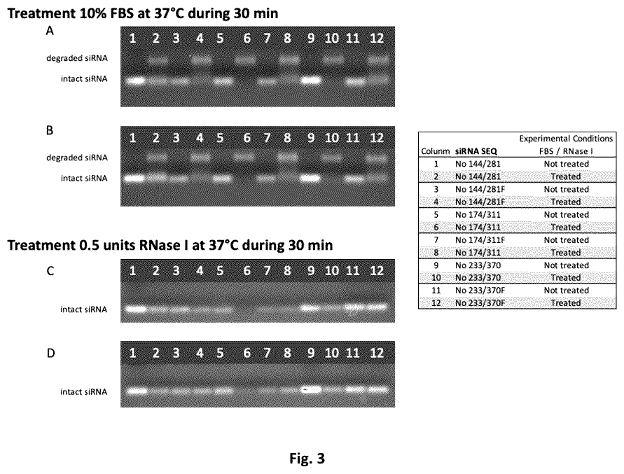

Stability of chemically modified siRNAs against dopamine-beta-hydroxylase: siRNA sequences to be used in the study were thaw and incubated at 37 QC during up to 120 min with cell serum-free culture medium added with RNase I (0.25 or 0.50 Units). In contrast to non-modified (natural) siRNAs, chemically modified siRNAs against dopamine-beta-hydroxylase show a significant resistance to degradation in culture medium containing RNAse I (0.50 Units) for up to 120 min (FIG. 2). These chemically modified siRNAs against dopamine-beta-hydroxylase retain their capacity in RISC engagement and downregulation of dopamine-beta-hydroxylase mRNA expression (FIG. 3).

### Example 3

Dopamine-beta-hydroxylase gene silencing: Total RNA was isolated and purified using the SV Total RNA Isolation System (Promega, USA) according to manufacturer's instructions. RNA quality and concentration were verified in the NanoDrop ND1000 Spectrophotometer (Thermo Scientific, USA), and RNA integrity and genomic DNA contamination were evaluated by agarose gel electrophoresis. Total RNA (1 μg) was converted into cDNA using the Maxima Scientific First Strand cDNA Synthesis Kit for RT-qPCR (Thermo Scientific, USA), according to instructions. The following protocol was used: 1^{st} step, 10 min at 25° C.; 2^{nd} step, 15 min at 50° C.; 3^{rd} step, 5 min at 85° C. cDNA was used for qPCR analysis using Maxima SYBR Green qPCR Master Mix (Thermo Scientific, USA) in the StepOnePlus instrument (Applied Biosystems, USA). Primer Assay for dopamine-beta-hydroxylase and for the endogenous control gene GAPDH (Quiagen, Germany) were used. The qPCR reaction was performed in 96-well PCR plates (Sarstedt, Germany) as follows: one cycle of 10 min at 95° C., followed by 40 PCR cycles at 95° C. 15 s and 60° C. 60 s. A melting curve was made immediately after the qPCR, to demonstrate the specificity of the amplification. No template controls were always evaluated for each target gene. Quantification cycle (Cq) values were generated automatically by the StepOnePlus 2.3 Software and the ratio of the target gene was expressed in comparison to the endogenous control gene GAPDH. Real-time PCR efficiencies were found to be between 90% and 110%.

### Example 4

Dopamine-beta-hydroxylase expression: Cells were rinsed twice with cold phosphate-buffered saline (PBS) and incubated with 100 μL RIPA lysis buffer (154 mM NaCl, 65.2 mM TRIZMA base, 1 mM EDTA, 1% NP-40 (IGEPAL), 6 mM sodium deoxycholate) containing protease inhibitors: 1 mM PMSF, 1 μg/mL leupeptine and 1 μg/mL aprotinin; and phosphatase inhibitors: 1 mM Na 3 VO 4 and 1 mM NaF. Cells were scraped and briefly sonicated. Equal amounts of total protein (30 μg) were separated on a % SDS-polyacrylamide gel and electrotransfered to a nitrocellulose membrane in Tris-Glycine transfer buffer containing 20% methanol. The transblot sheets were blocked in 5% non-fat dry milk in Tris-buffered saline (TBS) for 60 min and then incubated overnight, at 4° C., with the antibodies against dopamine-beta-hydroxylase and GAPDH, diluted in 2.5% non-fat dry milk in TBS-Tween 20 (0.1% vol/vol). The immunoblots were subsequently washed and incubated with fluorescently-labelled secondary antibodies (1:20,000; AlexaFluor 680, Molecular Probes) for 60 min at room temperature (RT) and protected from light. Membranes were washed and imaged by scanning at both 700 nm and 800 nm with an Odyssey Infrared Imaging System (LI-COR Biosciences).

### Example 5

Dopamine-beta-hydroxylase activity: Cells were rinsed twice with cold phosphate-buffered saline (PBS) and pre-incubated for 15 minutes in Hanks media at 37° C. Hanks media had the following composition (in mM): NaCl 140, KCl 5, MgSO₄-7H₂O 0.8, K2HPO₄ 0.33, KH₂PO₄ 0.44, MgCl₂ 0.6H₂O 1.0, CaCl₂ 0.025, Tris-HCl 9.75, pH 7.4. The reaction was initiated by adding 3 μM L-dihydroxyphenylalanine plus ascorbic acid (at 1 mM; co-factor) to the Hanks media in the absence and the presence of 1 μM nepicastat, for 360 minutes. During the pre-incubation and the incubation, cells were continuously shacked and maintained at 37° C. in a water bath. The reaction was stopped through the rapid removal of the incubation solution through aspiration with a Pasteur pipette, followed by a quick wash with Hanks media. Subsequently, cells were added with 0.2 M perchloric acid and stored at 4° C. for 24 hours. Thereafter, 900 μl of perchloric acid in which the cells were kept was used for the quantification of noradrenaline by means of high-pressure liquid chromatography with electrochemical detection (HPLC-EC).

### Example 6

In vivo dopamine-beta-hydroxylase inhibition and experimental glaucoma studies: Wistar rats were delivered with 4-6 weeks of age and used for the treatment with dopamine-beta-hydroxylase inhibitors or siRNAs against dopamine-beta-hydroxylase at least one week of quarantine. All animal interventions were conducted according to the European Directive 86/609, and the guidelines “Guide for the Care and Use of Laboratory Animals”, 7th edition, 1996, Institute for Laboratory Animal Research (I LAR), Washington, DC.

### Example 7

The induction of high intra ocular pressure (experimental glaucoma) can be obtained according with the procedures previously described by Ishikawa et al. (Ishikawa, Yoshitomi, Zorumski & Izumi, 2015), namely the topical application of hydroxyanmphetamine or by means of elevated hydrostatic pressure in an in vitro model with retinal organotypic cultures (Madeira et al., 2015).

While the foregoing disclosure provides a general description of the subject matter encompassed within the scope of the present disclosure, including methods, as well as the best mode thereof, of making and using this disclosure, the following examples are provided to further enable those skilled in the art to practice this disclosure and to provide a complete written description thereof. However, those skilled in the art will appreciate that the specifics of these examples should not be read as limiting on the disclosure, the scope of which should be apprehended from the claims and equivalents thereof appended to this disclosure. Various further aspects and embodiments of the present disclosure will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

Unless context dictates otherwise, the descriptions and definitions of the features set out above are not limited to any particular aspect or embodiment of the disclosure and apply equally to all aspects and embodiments which are described. The disclosure is further described in the following non-limiting examples.

Additional aspects of the invention will be apparent to those skilled in the art, or may be learned from the practice of the invention. The objects and advantages of the invention may be realized and attained by means of the instrumentalities and combinations particularly pointed out in the appended claims.

### REFERENCES

1. Ahmed F A, Hegazy K, Chaudhary P, & Sharma S C (2001). Neuroprotective effect of alpha(2) agonist (brimonidine) on adult rat retinal ganglion cells after increased intraocular pressure. Brain Res 913: 133-139.
2. Brummelkamp T R, Bernards R, & Agami R (2002). A system for stable expression of short interfering RNAs in mammalian cells. Science 296: 550-553.
3. Burke 0.1, Kharlamb A, Shan T, Runde E, Padillo E, Manlapaz C, et al. (1995). Adrenergic and imidazoline receptor-mediated responses to UK-14,304-18 (brimonidine) in rabbits and monkeys. A species difference. Ann N Y Acad Sci 763: 78-95.
4. Campochiaro P A (2006). Potential applications for RNAi to probe pathogenesis and develop new treatments for ocular disorders. Gene Ther 13: 559-562.
5. Coleman A L, & Miglior S (2008). Risk factors for glaucoma onset and progression. Surv Ophthalmol 53 Suppl1: S3-10.
6. Davis B M, Crawley L, Pahlitzsch M, Javaid F, & Cordeiro M F (2016). Glaucoma: the retina and beyond. Acta Neuropathol 132: 807-826.
7. Davis M E (2009). The first targeted delivery of siRNA in humans via a self-assembling, cyclodextrin polymer-based nanoparticle: from concept to clinic. Mol Pharm 6: 659-668.
8. Elbashir S M, Harborth 0.1, Lendeckel W, Yalcin A, Weber K, & Tuschl T (2001). Duplexes of 21-nucleotide RNAs mediate RNA interference in cultured mammalian cells. Nature 411: 494-498.
9. Fire A, Xu S, Montgomery M K, Kostas S A, Driver S E, & Mello C C (1998). Potent and specific genetic interference by double-stranded RNA in Caenorhabditis elegans. Nature 391: 806-811.
10. Frezzotti P, Giorgio A, Motolese I, De Leucio A, Iester M, Motolese E, et al. (2014). Structural and functional brain changes beyond visual system in patients with advanced glaucoma. PLoS One 9: e105931.
11. Frezzotti P, Giorgio A, Toto F, De Leucio A, & De Stefano N (2016). Early changes of brain connectivity in primary open angle glaucoma. Hum Brain Mapp 37: 4581-4596.
12. Fudemberg Si, Batiste C, & Katz U (2008). Efficacy, safety, and current applications of brimonidine. Expert Opin Drug Saf 7: 795-799.
13. Greenfield D S, Liebmann J M, & Ritch R (1997). Brimonidine: a new alpha2-adrenoreceptor agonist for glaucoma treatment. J Glaucoma 6: 250-258.
14. Gupta S K, Agarwal R, Galpalli N D, Srivastava S, Agrawal S S, & Saxena R (2007). Comparative efficacy of pilocarpine, timolol and latanoprost in experimental models of glaucoma. Methods Find Exp Clin Pharmacol 29: 665-671.
15. Hannon G J (2002). RNA interference. Nature 418: 244-251.
16. Harborth J, Elbashir S M, Bechert K, Tuschl T, & Weber K (2001). Identification of essential genes in cultured mammalian cells using small interfering RNAs. J Cell Sci 114: 4557-4565.
17. Hey J A, Gherezghiher T, & Koss M C (1985). Studies on the mechanism of clonidine-induced mydriasis in the rat. Naunyn Schmiedebergs Arch Pharmacol 328: 258-263.
18. Hsu W H, Lee P, & Betts D M (1981). Xylazine-induced mydriasis in rats and its antagonism by alpha-adrenergic blocking agents. J Vet Pharmacol Ther 4: 97-101.
19. Ishikawa M, Yoshitomi T, Zorumski C F, & Izumi Y (2015). Experimentally Induced Mammalian Models of Glaucoma. Biomed Res Int 2015: 281214.
20. Lee N S, Dohjima T, Bauer G, Li H, Li M-J, Ehsani A, et al. (2001). Expression of small interfering RNAs targeted against HIV-1 rev transcripts in human cells. Nature Biotechnology 19: 500-505.
21. Lee N S, Dohjima T, Bauer G, Li H, Li M-J, Ehsani A, et al. (2002). Expression of small interfering RNAs targeted against HIV-1 rev transcripts in human cells. Nature Biotechnology 20: 500-505.
22. Madeira M H, Elvas F, Boia R, Goncalves F Q, Cunha R A, Ambrosio A F, et al. (2015). Adenosine A2AR blockade prevents neuroinflammation-induced death of retinal ganglion cells caused by elevated pressure. J Neuroinflammation 12: 115.
23. Martinez T, Gonzalez M V, Roehl I, Wright N, Paneda C, & Jimenez Al (2014). In vitro and in vivo efficacy of SYL040012, a novel siRNA compound for treatment of glaucoma. Mol Ther 22: 81-91.
24. Miyagishi M, & Taira K (2002). U6 promoter-driven siRNAs with four uridine 3′ overhangs efficiently suppress targeted gene expression in mammalian cells. Nature Biotechnology 19: 497-500.
25. Nucci C, Martucci A, Cesareo M, Mancino R, Russo R, Bagetta G, et al. (2013). Brain involvement in glaucoma: advanced neuroimaging for understanding and monitoring a new target for therapy. Curr Opin Pharmacol 13: 128-133.
26. Okamura T, Fujioka H, & Ayajiki K (2002). Effects of nipradilol on alpha-adrenoceptor function in ocular arteries. Pharmacology 65: 110-118.
27. Paddison P J, Caudy A A, Bernstein E, Hannon G J, & Conklin D S S (2002). hort hairpin RNAs (shRNAs) induce sequence-specific silencing in mammalian cells. Genes Dev 16: 948-958.
28. Paul C P, Good P D, Winer I, & Engelke D R (2002). Effective Expression of Small Interfering RNA in human cells. Nature Biotechnology 19: 505-508.
29. Prokofyeva E, & Zrenner E (2012). Epidemiology of major eye diseases leading to blindness in Europe: a literature review. Ophthalmic Res 47: 171-188.
30. Quigley H A (2011). Glaucoma. Lancet 377: 1367-1377.
31. Rittenhouse K D, & Pollack G M (2000). Pharmacodynamics of beta-blocker modulation of aqueous humor production. Exp Eye Res 70: 429-439.
32. Seki M, Tanaka T, Matsuda H, Togano T, Hashimoto K, Ueda 0.1, et al. (2005). Topically administered timolol and dorzolamide reduce intraocular pressure and protect retinal ganglion cells in a rat experimental glaucoma model. Br J Ophthalmol 89: 504-507.
33. Sui G, Soohoo C, Affar E B, Gay F, Shi Y, Forrester W C, et al. (2002). A DNA vector-based RNAi technology to suppress gene expression in mammalian cells. Proc Natl Acad Sci USA 99: 5515-5520.
34. Tuschl T, Sharp P A, & Bartel D P (1998). Selection in vitro of novel ribozymes from a partially randomized U2 and U6 snRNA library. EMBO J 117: 2637-2650.
35. Webers C A, Beckers R I, Nuijts R M, & Schouten J S (2008). Pharmacological management of primary open-angle glaucoma: second-line options and beyond. Drugs Aging 25: 729-759.
36. Wheeler L A, & Woldemussie E (2001). Alpha-2 adrenergic receptor agonists are neuroprotective in experimental models of glaucoma. Eur J Ophthalmol 11 Suppl 2: S30-35.
37. Xia H, Mao Q, Paulson H L, & Davidson B L (2002). siRNA-mediated gene silencing in vitro and in vivo. Nat Biotechnol 20: 1006-1010.
38. Yu J Y, DeRuiter S L, & Turner D L (2002). RNA interference by expression of short-interfering RNAs and hairpin RNAs in mammalian cells. Proc Natl Acad Sci USA 99: 6047-6052.

1. An isolated or synthetic short interfering nucleic acid—(siNA)—molecule, wherein said molecule comprises a nucleic acid sequence selected from the group consisting of SEQ ID No 144, SEQ ID No 174, SEQ. ID No 233, SEQ ID No 235, SEQ ID No 239, SEQ ID No 250, SEQ ID No 265, SEQ ID No 269, SEQ ID No 281, SEQ ID No 311, SEQ ID No 370, SEQ ID No 372, SEQ ID No 376, SEQ ID No 387, SEQ ID No 402, SEQ ID No 406, and sequences comprising at least 18 contiguous nucleotides differing by no more than 4 nucleotides from the nucleotide sequence, and wherein said siNA molecule reduces expression of the dopamine-beta-hydroxylase—(DBH)—gene in a cell.

2. The siNA molecule according to claim 1, wherein said molecule comprises a nucleic acid sequence differing by no more than 3 nucleotides from the nucleotide sequence.

3. The siNA molecule according to claim 1, wherein said molecule comprises a nucleic acid sequence differing by no more than 2 nucleotides from the nucleotide sequence.

4. (canceled)

5. The siNA molecule according to claim 1, wherein said molecule is between 19 and 25 base pairs in length.

6. (canceled)

7. The siNA molecule according to claim 1, wherein said molecule comprises a nucleic acid sequence selected from the group consisting of SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ ID No 248, SEQ ID No 250, SEQ ID No 265, SEQ ID No 269, SEQ. ID No 279, SEQ ID No 281, SEQ ID No 282, SEQ ID No 287, SEQ ID No 288, SEQ ID No 290, SEQ ID No 291, SEQ. ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

8. The siNA molecule according to claim 1, wherein siNA is selected from dsRNA, siRNA or shRNA.

9. (canceled)

10. The siNA molecule according to claim 1, wherein siNA comprises 5′ and/or 3′ overhangs.

11. The siNA molecule according to claim 1, wherein siNA comprises at least one chemical modification.

12-13. (canceled)

14. A double stranded ribonucleic acid (dsRNA) agent for inhibiting expression of DBH-gene in a cell, wherein the dsRNA agent comprises a sense strand and an antisense strand forming a double stranded region, wherein the sense strand comprises a nucleic acid sequence selected from the group consisting of SEQ ID No 144, SEQ ID No 174, SEQ ID No 233, SEQ ID No 235, SEQ ID No 239, SEQ ID No 250, SEQ ID No 265, SEQ ID No 269, and sequences comprising at least 18 contiguous nucleotides differing by no more than 4 nucleotides from the nucleotide sequence, and wherein the antisense strand comprises a nucleic acid sequence selected from the group consisting of SEQ ID No 281, SEQ. ID No 311, SEQ ID No 370, SEQ ID No 372, SEQ ID No 376, SEQ ID No 387, SEQ ID No 402, SEQ ID No 406, and sequences comprising at least 18 contiguous nucleotides differing by no metre than 4 nucleotides from the nucleotide sequence.

15. The dsRNA agent according to claim 14, wherein said sense strand or antisense strand comprises a nucleic acid sequence differing by no more than 3 nucleotides from the nucleotide sequence.

16. The dsRNA agent according to claim 14, wherein said sense strand or antisense strand comprises a nucleic acid sequence differing by no more than 2 nucleotides from the nucleotide sequence.

17. (canceled)

18. The dsRNA agent according to claim 14, wherein the sense strand is selected from the group consisting of SEQ ID No 142, SEQ ID No 144, SEQ ID No 145, SEQ ID No 150, SEQ ID No 151, SEQ ID No 153, SEQ ID No 154, SEQ ID No 157, SEQ ID No 158, SEQ ID No 172, SEQ ID No 174, SEQ ID No 175, SEQ ID No 181, SEQ ID No 183, SEQ ID No 184, SEQ ID No 193, SEQ ID No 195, SEQ ID No 196, SEQ ID No 197, SEQ ID No 200, SEQ ID No 201, SEQ ID No 210, SEQ ID No 211, SEQ ID No 231, SEQ ID No 233, SEQ ID No 234, SEQ ID No 235, SEQ ID No 239, SEQ No 248, SEQ ID No 250, SEQ ID No 265 and SEQ ID No 269, and wherein the anti-sense strand is selected from the group consisting of SEQ ID No 279, SEQ ID No 281, SEQ ID No 282, SEQ ID No 287, SEQ ID No 288, SEQ ID No 290, SEQ ID No 291, SEQ ID No 294, SEQ ID No 295, SEQ ID No 309, SEQ ID No 311, SEQ ID No 312, SEQ ID No 318, SEQ ID No 320, SEQ ID No 321, SEQ ID No 330, SEQ ID No 332, SEQ ID No 333, SEQ ID No 334, SEQ ID No 337, SEQ ID No 338, SEQ ID No 347, SEQ ID No 348, SEQ ID No 368, SEQ ID No 370, SEQ ID No 371, SEQ ID No 372, SEQ ID No 376, SEQ ID No 385, SEQ ID No 387, SEQ ID No 402 and SEQ ID No 406.

19. A vector comprising a molecule described in claim 1.

20. A liposome, microsphere, nanoparticle or capsule comprising a molecule described in claim 1.

21. A pharmaceutical composition comprising at least one siNA molecule according to claim 1 and a pharmaceutically acceptable carrier.

22. The composition according to claim 21, comprising a second active ingredient for the treatment of glaucoma.

23. The composition according to claim 22, wherein said second active ingredient is selected from the group consisting of: an alpha adrenoceptor agonist, a beta adrenoceptor blocker, a carbonic anhydrase inhibitor, a muscarinic agonist, a prostaglandin analogue, a rho kinase inhibitor, and mixtures thereof.

24. (canceled)

25. A method for preventing or reversing progressive optical neuropathy in a subject, the method comprising administrating the siNA molecule siRNA according to claim 1 to the subject.

26. The method according to claim 25, wherein the progressive optical neuropathy is selected from the group consisting of diabetic retinopathy, infections, inflammation, uveitis and glaucoma.

27. The method according to claim 25, wherein the progressive optical neuropathy is open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome, or uveitic glaucoma.
